(19) United States
(12) Patent Application Publication
(10) Pub. No.: US 2010/0184047 A1
(43) Pub. Date: Jul. 22, 2010

(54) SCREENING AND THERAPEUTIC METHOD FOR NSCLC TARGETING THE CDCA8-AURKB COMPLEX

(75) Inventors: Yusuke Nakamura, Tokyo (JP); Yataro Daigo, Tokyo (JP); Shuichi Nakatsuru, Kanagawa (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ONCOTHERAPY SCIENCE, INC., Kanagawa (JP)

(21) Appl. No.: 12/593,265

(22) PCT Filed: Mar. 27, 2008
(86) PCT No.: PCT/JP2008/056657

§ 371 Date: Mar. 24, 2010

Related U.S. Application Data
(60) Provisional application No. 60/909,347, filed on Mar. 30, 2007.

(51) Int. Cl. C12Q 1/68; G01N 33/53

(52) U.S. Cl. 435/6; 435/7.1

(57) ABSTRACT

The present invention is based on the observation that the co-activation of CDCA8 and AURKB, and their cognate interactions, play a significant role in lung-cancer progression. Accordingly, inhibiting the formation of the CDCA8-AURKB complex finds utility in the treatment of non-small-cell lung cancer. The present invention also provides methods for identifying for compounds suitable for the treatment and/or prevention non-small-cell lung cancer, using, for example, the transcriptional regulatory region of the CDCA8 or AURKB gene, as well as diagnostic and prognostic methods that utilize the expression levels of CDCA8 and/or AURKB as a determining index.

This application claims the benefit of U.S. Provisional Application Ser. No. 60/909,347 filed Mar. 30, 2007, the contents of which are hereby incorporated by reference in their entirety.

### FIELD OF THE INVENTION

The present invention relates to the field of biological science, more specifically to the field of cancer therapy. In particular, the present invention relates to screening methods that use the interaction between CDCA8 and AURKB as an index. Agents suited to the treatment and prevention of cancer, in particular non-small-cell lung cancer (NSCLC), can be identified through such methods. Furthermore, given that the co-activation of CDCA8 and AURKB, and their cognate interactions, are demonstrated herein to play a significant role in lung-cancer progression, the present invention also relates to methods of treating and preventing non-small-cell lung cancer that involve inhibition of the formation CDCA8-AURKB complex and methods of assessing the prognosis of an NSCLC patient using the expression levels of CDCA8 and/or AURKB as an index.

### BACKGROUND OF THE INVENTION

Lung cancer is one of the most common cancers in the world, and non-small-cell lung cancer (NSCLC) accounts for nearly 80% of those cases (Greenlee R T., et al. CA Cancer J Clin. 2001 Jan.-Feb.; 51(1):15-36). Although many genetic alterations involved in development and progression of lung cancer have been reported, the precise molecular mechanisms still remain unclear (Sozzi G. Eur J Cancer. 2001 October; 37 Suppl 7:S63-73). Over the last decade newly-developed cytotoxic agents, including paclitaxel, docetaxel, gemcitabine, and vinorelbine, have emerged to offer multiple therapeutic choices for patients with advanced NSCLC. However, these regimens provide only modest survival benefits as compared with cisplatin-based therapies (Schiller J H, et al., N Engl J Med. 2002 Jan. 10; 346(2):92-8; Kelly K, et al., J Clin Oncol. 2001 Jul. 1; 19(13):3210-8).

In addition to cytotoxic drug therapy, therapies involving molecular-targeted agents, such as monoclonal antibodies against VEGF (i.e., bevacizumab/anti-VEGF) or EGFR (i.e., cetuximab/anti-EGFR) as well as inhibitors for EGFR tyrosine kinase (i.e., gefitinib and erlotinib) have been developed, and applied in clinical practice (Perrone F, et al., Curr Opin Oncol. 2005 March; 17(2):123-9). However, as each of the new therapies can provide survival benefits to a small subset of the patients (Thatcher N, et al., Lancet. 2005 Oct. 29-Nov. 4; 366(9496):1527-37; Shepherd F A, et al., N Engl J Med. 2005 Jul. 14; 353(2):123-32), new therapeutic strategies are eagerly anticipated. In particular, there is a need in the art for more effective molecular-targeted agents applicable to the great majority of patients with less toxicity.

Systematic analysis of expression levels of thousands of genes using cDNA microarray technology provides an effective approach for the identification of unknown molecules involved in carcinogenic pathways, and can be used to effectively screen candidate target molecules for the development of novel therapeutics and diagnostics. Attempts to isolate novel potential molecular targets for diagnosis, treatment and/or prevention of NSCLC, for example by analyzing genome-wide expression profiles of 101 lung cancer tissue samples on a cDNA microarray containing 27,648 genes (Kikuchi T, et al. Oncogene. 2003 Apr. 10; 22(14):2192-205; Kakiuchi S, et al., Mol Cancer Res. 2003 May; 1(7):485-99; Kakiuchi S, et al., Hum Mol Genet. 2004 Dec. 15; 13(24):3029-43. Epub 2004 Oct. 20; Kikuchi T, et al., Int J Oncol. 2006 April; 28(4):799-805; Taniwaki M, et al., Int J Oncol. 2006 September; 29(3):567-75), are ongoing.

In particular, to verify the biological and clinicopathological significance of the respective gene products, a screening system that combines the tumor-tissue microarray analysis of clinical lung-cancer materials and RNA interference has been established (RNAi) technique (Suzuki C, et al., Cancer Res. 2003 Nov. 1; 63(20:7038-41; Ishikawa N, et al. Clin Cancer Res. 2004 Dec. 15; 10(24):8363-70; Kato T, et al., Cancer Res. 2005 Jul. 1; 65(13):5638-46; Furukawa C, et al., Cancer Res. 2005 Aug. 15; 65(16):7102-10; Ishikawa N, et al., Cancer Res. 2005 Oct. 15; 65(20):9176-84; Suzuki C, et al., Cancer Res. 2005 Dec. 15; 65(24):11314-25; Ishikawa N, et al., Cancer Sci. 2006 August; 97(8):737-45; Takahashi K, et al., Cancer Res. 2006 Oct. 1; 66(19):9408-19; Hayama S, et al., Cancer Res. 2006 Nov. 1; 66(21):10339-48).

This systematic approach resulted in the identification of 642 up-regulated genes and 806 down-regulated genes as diagnostic markers and therapeutic targets for NSCLC (See WO 2004/31413, the contents of which are incorporated by reference herein). One in particular was cell division associated 8 (CDCA8), a gene that was shown to be frequently over-expressed in primary lung cancers and essential for growth/survival and malignant nature of lung-cancer cells.

Recently, CDCA8 was identified as a new component of the vertebrate chromosomal passenger complex. CDCA8 was suggested to be phosphorylated in vitro by aurora kinase B (AURKB), though the precise phosphorylated sites and its functional importance in cancer cells, as well as in normal mammalian cells, remains unclear (Sampath S C, et al., Cell. 2004 Jul. 23; 118(2):187-202; Gassmann R, et al., J Cell Biol. 2004 Jul. 19; 166(2):179-91. Epub 2004 Jul. 12). The chromosome passenger complex is composed of at least four proteins; AURKB, inner centromere protein antigens 135/155 kDa (INCENP), BIRC5/Survivin, and CDCA8 (Vagnarelli P & Earnshaw W C. Chromosoma. 2004 November; 113(5):211-22. Epub 2004 Sep. 4), each of which demonstrates a dynamic cellular localization pattern during mitosis (Higuchi T & Uhlmann F. Nature. 2003 Dec. 18; 426(6968):780-1).

Since several mitotic functions of the chromosomal passenger complex have been reported, such as the regulation of metaphase chromosome alignment, sister chromatid resolution, spindle checkpoint signaling, and cytokinesis (Carmena M & Earnshaw W C. Nat Rev Mol Cell Biol. 2003 November; 4(11):842-54), this complex may be categorized as a mitotic regulator. Activation of AURKB and BIRC5 was reported in some of human cancers (Bischoff J R, et al., EMBO J. 1998 Jun. 1; 17(11):3052-65; Branca M, et al., Am J Clin Pathol. 2005 July; 124(1):113-21), and many other mitotic and/or cell cycle regulators are also aberrantly expressed in tumor cells and are considered to be targets for development of promising anti-cancer drugs.

In fact, CDK inhibitors (such as flavopiridol, UCN-01, E7070, R-Roscovitine, and BMS-387032), specific KIF11 inhibitor (monastrol), and histone deacetyltransferase (HDAC) inhibitors have been revealed to possess anti-cancer activity and have therefore been applied in preclinical or clinical phases (Blagden S & de Bono J. Curr Drug Targets. 2005 May; 6(3):325-35; Bergnes G, et al., Curr Top Med Chem. 2005; 5(2):127-45; Mork C N, et al., Curr Pharm Des. 2005; 11(9): 1091-104).

Herein is described the novel mechanism of the oncogenic activation of CDCA8 by AURKB that is important for lung-cancer growth and progression. Also demonstrated herein is the discovery that functional inhibition of the CDCA8/AURKB interaction can lead to potential strategies for treatment of lung-cancer patients.

### BRIEF SUMMARY OF THE INVENTION

As discussed in greater detail herein, genome-wide gene-expression analysis of lung carcinomas resulted in detection in the great majority of lung-cancer samples tested of the co-transactivation of cell-division-associated 8 (CDCA8) (GenBank Accession No. NM_{—}018101; SEQ ID NO: 2 encoded by SEQ ID NO: 1) and aurora kinase B (AURKB) (GenBank Accession No. NM_{—}004217; SEQ ID NO: 4 encoded by SEQ ID NO: 3), each of which is considered to be a component of the vertebrate chromosomal passenger complex.

In addition, immunohistochemical analysis of lung-cancer tissue microarrays demonstrated that over-expression of CDCA8 and AURKB is significantly associated with poor prognosis of lung-cancer patients.

In particular, AURKB was shown to directly phosphorylate CDCA8 at the Ser-154, Ser-219, Ser-275, and Thr-278 residues, and appeared to stabilize the CDCA8 protein in cancer cells. Suppression of CDCA8 expression using siRNA against CDCA8 significantly suppressed the growth of lung cancer cells.

Furthermore, functional inhibition of the interaction between CDCA8 and AURKB, using, for example, a cell-permeable peptide corresponding to a 20 amino-acid sequence fragment of CDCA8 (11R-CDCA8₂₆₁₋₂₈₀) (SEQ ID NO: 5), which includes two AURKB phosphorylation sites, significantly reduced the phosphorylation of CDCA8 and resulted in the suppression of the growth of lung cancer cell. Taken together, the data herein suggest that the selective suppression of the CDCA8-AURKB pathway constitutes a promising therapeutic strategy for lung cancer patients.

It will be understood by those skilled in the art that one or more aspects of the present invention can meet certain objectives, while one or more other aspects can meet certain other objectives. Each objective may not apply equally, in all its respects, to every aspect of this invention. As such, the following objects may be viewed in the alternative with respect to any one aspect of this invention.

In view of the foregoing, it is an object of the present invention to provide methods of screening for a compound suitable for the treatment and/or prevention of NSCLC. Illustrative methods include the steps of:
(a) contacting an AURKB polypeptide or functional equivalent thereof with a CDCA8 polypeptide or functional equivalent thereof in the presence of a test compound;
(b) assaying the binding between the polypeptides of step (1); and
(c) selecting the test compound that inhibits the binding between the polypeptides.

An exemplary functional equivalent of a CDCA8 polypeptide may have an amino acid sequence that corresponds to the AURKB binding domain, for example the amino acid sequence of SEQ ID NO: 5 (NIKKLSNRLAQICSSIRTHK). Likewise, an exemplary functional equivalent of an AURKB polypeptide may have an amino acid sequence that corresponds to the CDCA8 binding domain.

It is a further object of the present invention to provide methods of screening for a compound suitable for the treatment and/or prevention of NSCLC. An illustrative method includes the steps of:
(a) contacting an AURKB polypeptide or functional equivalent thereof with a CDCA8 polypeptide or functional equivalent thereof in the presence of a test compound;
(b) assaying the phosphorylation of the CDCA8 polypeptide by the AURKB polypeptide or the amount of the CDCA8 polypeptide; and
(c) selecting the test compound that inhibits the phosphorylation of the CDCA8 polypeptide or reduces the amount of the CDCA8 polypeptide.

An exemplary functional equivalent of a CDCA8 polypeptide may have an amino acid sequence that includes the phosphorylation site, including, for example the Ser-154, Ser-219, Ser-275, and/or Thr-278 residues of the amino acid sequence of SEQ ID NO: 2. Likewise, an exemplary functional equivalent of an AURKB polypeptide may have an amino acid sequence that corresponds to the kinase domain.

It is a further object of the present invention to provide methods for treating and/or preventing NSCLC in a subject, such methods involving the administration of a compound that is obtained by the screening methods of the present invention described above.

It is a further object of the present invention further to provide a kit for screening for a compound suitable for the treatment and/or prevention of NSCLC. Such a kit preferably includes, at a minimum, the following components:
a: an AURKB polypeptide or functional equivalent thereof, and
b: a CDCA8 polypeptide or functional equivalent thereof.

It is a further object of the present invention to provide methods for treating and/or preventing NSCLC in a subject, such methods including the step of administering to the subject an siRNA composition containing an siRNA that reduces the expression of an AURKB gene, wherein the siRNA has the nucleotide sequence selected from the group consisting of SEQ ID NO: 33, 59 and 60, in the sense strand as a target sequence. Such an siRNA preferably has the following general formula:

5′-[A]-[B]-[A′]-3′,
wherein [A] is a ribonucleotide sequence corresponding to a sequence selected from the group consisting of SEQ ID NO: 33, 59 and 60; [B] is a ribonucleotide sequence composed of 3 to 23 nucleotides; and [A′] is a ribonucleotide sequence complementary to [A].

It is a further object of the present invention to provide methods for treating and/or preventing NSCLC in a subject by administering a CDCA8 mutant having dominant negative effect, or a polynucleotide encoding such a mutant. Such a CDCA8 mutant may have an amino acid sequence that includes an AURKB binding region, e.g. the part of a CDCA8 protein that includes phosphorylation sites, Ser-154, Ser-219, Ser-275, and Thr-278, all of which are phosphorylated by AURKB. In a preferred embodiment, the CDCA8 mutant has the amino acid sequence of SEQ ID NO: 5. The CDCA8 mutant may alternatively have the following general formula: [R]-[D], wherein [R] is a membrane transducing agent, and [D] is a polypeptide having the amino acid sequence of SEQ ID NO: 5. The membrane transducing agent can be selected from group consisting of;

| | |
|---|---|
| poly-arginine; | |
| Tat/ | |
| RKKRRQRRR/; | SEQ ID NO: 6 |
| Penetratin/ | |
| RQIKIWFQNRRMKWKK/; | SEQ ID NO: 7 |
| Buforin II/ | |
| TRSSRAGLQFPVGRVHRLLRK/; | SEQ ID NO: 8 |
| Transportan/ | |
| GWTLNSAGYLLGKINLKALAALAKKIL/; | SEQ ID NO: 9 |
| MAP (model amphipathic peptide)/ | |
| KLALKLALKALKAALKLA/; | SEQ ID NO: 10 |
| K-FGF/ | |
| AAVALLPAVLLALLAP/; | SEQ ID NO: 11 |
| Ku70/ | |
| VPMLK/; | SEQ ID NO: 12 |
| Ku70/ | |
| PMLKE/; | SEQ ID NO: 13 |
| Prion/ | |
| MANLGYWLLALFVTMWTDVGLCKKRPKP/; | SEQ ID NO: 14 |
| pVEC/ | |
| LLIILRRRIRKQAHAHSK/; | SEQ ID NO: 15 |
| Pep-1/ | |
| KETWWETWWTEWSQPKKKRKV/; | SEQ ID NO: 16 |
| SynB1/ | |
| RGGRLSYSRRRFSTSTGR/; | SEQ ID NO: 17 |
| Pep-7/ | |
| SDLWEMMMVSLACQY/; | SEQ ID NO: 18 |
| and | |
| HN-1/ | |
| TSPLNIHNGQKL/. | SEQ ID NO: 19 |

It is yet a further object of the present invention to provide a double-stranded molecule composed of a sense strand and an antisense strand, wherein the sense strand is a ribonucleotide sequence corresponding to an AURKB target sequence, and the antisense strand is a ribonucleotide sequence which is complementary to the sense strand, such that the sense strand and the antisense strand hybridize to each other to form a double-stranded molecule that, when introduced into a cell expressing an AURKB gene, inhibits the expression of the gene. The double-stranded molecule may include an AURKB target sequence composed of at least about 10 contiguous nucleotides selected from the nucleotide sequence of SEQ ID NO: 33, 59 or 60. In a preferred embodiment, the AURKB target sequence contains from about 19 to about 25 contiguous nucleotides selected from the nucleotide sequence of SEQ ID NO: 3, or may alternatively be composed entirely of SEQ ID NO: 3.

The double-stranded molecule may also be a single ribonucleotide transcript composed of the sense strand and the antisense strand linked via an intervening single-strand, for example a single stranded ribonucleotide sequence. Such a double-stranded molecule may optionally contain a 3′ overhang. The double-stranded molecule is typically an oligonucleotide that is less than about 100 nucleotides in length, preferably less than about 75 nucleotides in length, more preferably less than about 50 nucleotides in length, even more preferably less than about 25 nucleotides in length. In a preferred embodiment, the double-stranded molecule is an oligonucleotide between about 19 and about 25 nucleotides in length.

It is a further object of the present invention to provide a vector that encodes the double-stranded molecule of the invention described above. The vector may encode a transcript having a secondary structure that includes the sense strand and the antisense strand. The transcript may further include an intervening single-strand, for example, a single-stranded ribonucleotide sequence, linking the sense strand and the antisense strand.

Alternatively, the vector may encode both of the sense strand and the antisense strand, to form the double-stranded molecule by expression of both strands as two transcripts. Further, vectors may encode a combination of the sense strand and the an antisense strand are also provided.

It is yet a further object of the present invention to provide a vector containing a polynucleotide composed of a combination of a sense strand nucleic acid and an antisense strand nucleic acid, wherein the sense strand nucleic acid has the nucleotide sequence selected from the group consisting of SEQ ID NO: 33, 59 and 60, and the antisense strand nucleic acid has a sequence complementary to the sense strand.

The polynucleotide may have the general formula of:

5′-[A]-[B]-[A′]-3′,
wherein [A] is a nucleotide sequence selected from the group consisting of SEQ ID NOs: 33, 59 and 60; [B] is a nucleotide sequence consisting of 3 to 23 nucleotides; and [A′] is a nucleotide sequence complementary to [A].

It is yet a further object of the present invention to provide compositions for treating or preventing NSCLC, such compositions including a pharmaceutically effective amount of an siRNA against an AURKB gene. The siRNA may include a sense strand having the nucleotide sequence selected from the group consisting of SEQ ID NO: 33, 59 and 60 as the target sequence.

It is yet a further object of the present invention to provide compositions for treating or preventing NSCLC, such compositions including as an active ingredient a pharmaceutically effective amount of a compound selected by the screening methods of the present invention described above, and a pharmaceutically acceptable carrier.

It is yet a further object of the present invention to provide compositions for treating or preventing NSCLC, such compositions including a pharmaceutically effective amount of a CDCA8 mutant of the present invention.

It is yet a further object of the present invention to provide methods of assessing an NSCLC prognosis, wherein the method includes the steps of:
(a) detecting the expression level of either CDCA8 and AURKB, or both, in a specimen collected from a subject whose NSCLC prognosis is to be assessed, and
(b) indicating a poor prognosis when an elevation in the expression level of either of CDCA8 and AURKB or both is detected.

The above method may also include the step of detecting the expression level of either CDCA8 or AURKB. The expression level may be detected by any one of the following methods:
(a) detecting the presence of an mRNA encoding the amino acid sequence of SEQ ID NO: 2 (CDCA8) or SEQ ID NO: 4 (AURKB),
(b) detecting the presence of a protein having the amino acid sequence of SEQ ID NO: 2 (CDCA8) or SEQ ID NO: 4 (AURKB), and
(c) detecting the biological activity of a protein having the amino acid sequence of SEQ ID NO: 2 (CDCA8) or SEQ ID NO: 4 (AURKB).

It is yet a further object of the present invention to provide kits for assessing an NSCLC prognosis, wherein the kit includes one or more components selected from the group consisting of:
(a) a reagent for detecting an mRNA encoding the amino acid sequence of SEQ ID NO: 2 (CDCA8) or SEQ ID NO: 4 (AURKB),
(b) a reagent for detecting a protein having the amino acid sequence of SEQ ID NO: 2 (CDCA8) or SEQ ID NO: 4 (AURKB), and
(c) a reagent for detecting the biological activity of a protein having the amino acid sequence of SEQ ID NO: 2 (CDCA8) or SEQ ID NO: 4 (AURKB).

It is yet a further object of the present invention to provide a method of screening for compounds for treating or preventing non-small cell lung cancer, the method including the steps of:
(a) contacting a test compound with a cell into which a vector containing the transcriptional regulatory region of CDCA8 or AURKB gene and a reporter gene that is expressed under the control of the transcriptional regulatory region has been introduced,
(b) measuring the expression level or activity of the reporter gene; and
(c) selecting a compound that reduces the expression level or activity of the reporter gene, as compared to a control.

In the method described above, the transcriptional regulatory region may include an E2F-1 motif.

It is yet a further object of the present invention to provide a kit for screening for a compound for treating or preventing NSCLC, the kit including the components of:
(a) a cell into which a vector containing the transcriptional regulatory region of CDCA8 or AURKB gene and a reporter gene that is expressed under the control of the transcriptional regulatory region has been introduced, and
(b) a reagent for measuring the expression level or activity of the reporter gene.

It is yet a further object of the present invention to provide a method for treating or preventing NSCLC in a subject, the method including the step of administering an inhibitor having at least one function selected from the group consisting of:

i. inhibiting a binding between CDCA8 and AURKB;

ii. inhibiting a phosphorylation of CDCA8 by AURKB; and

iii. inhibiting a transcription of either of CDCA8 and AURKB genes, or both.

It is yet a further object of the present invention to provide composition for treating or preventing NSCLC, the composition composed of a pharmaceutically effective amount of an inhibitor having at least one function selected from the group consisting of:

i. inhibiting a binding between CDCA8 and AURKB;

ii. inhibiting a phosphorylation of CDCA8 by AURKB; and

iii. inhibiting a transcription of either of CDCA8 and AURKB genes, or both.

These and other objects and features of the invention will become more fully apparent when the following detailed description is read in conjunction with the accompanying figures and/or examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention.

Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn there-from, alone or with consideration of the references incorporated herein.

Various aspects and applications of the present invention will become apparent to the skilled artisan upon consideration of the brief description of the drawings and the detailed description of the present invention and its preferred embodiments which follows:

### BRIEF DESCRIPTION OF THE DRAWINGS

FIG. 1. depicts the activation of CDCA8 and AURKB proteins in lung tumor samples.

Part A depicts the expression of CDCA8 and AURKB in clinical samples of 14 NSCLC (T) and corresponding normal lung tissues (N), examined by semi-quantitative RT-PCR. The Appropriate dilutions of each single-stranded cDNA were prepared from mRNAs of clinical lung-cancer samples, taking the level of beta-actin (ACTB) expression as a quantitative control.

Part B depicts the expression of the CDCA8 and AURKB proteins in 11 lung-cancer cell lines, examined by western-blot analysis.

Part C depicts the expression of CDCA8 in 23 normal human tissues, detected by northern-blot analysis.

Part D depicts the subcellular localization of endogenous CDCA8 (upper panels) and endogenous AURKB (lower panels) in LC319 cells, detected by rabbit polyclonal antibodies to CDCA8 or AURKB. Both were triple-stained with alpha-tubulin and DAPI (see Merged images). CDCA8 or AUKRB was stained at DAPI stained location.

FIG. 2. depicts the association of CDCA8 and AURKB over-expression with poor clinical outcomes in NSCLC.

Part A depicts the results of immunohistochemical evaluation of representative samples from surgically-resected SCC tissues, using anti-CDCA8 (upper panels) and anti-AURKB (lower panels) polyclonal antibodies on tissue microarrays (×100).

Part B depicts the results of Kaplan-Meier analysis of tumor-specific survival times according to expression of CDCA8 (upper left panel), AURKB (upper right panel) or combined of CDCA8 and AURKB (lower panel) on tissue microarrays.

FIG. 3 depicts the inhibition of growth of lung-cancer cells by siRNAs against CDCA8.

The results of western-blot analysis, depicting the gene knock-down effect on CDCA8 protein expression in LC319 cells by two si-CDCA8s (si-CDCA8-#1 and -#2) and two control siRNAs (si-EGFP and -Luciferase), are depicted in the upper panels. The results of colony formation assays (middle panels) and MTT assays (lower panel) of LC319 cells transfected with si-CDCA8s or control plasmids. Error bars represent the standard deviation of triplicate assays.

FIG. 4 depicts the transcriptional regulation of CDCA8 and AURKB by E2F-1.

Part A, (upper panel) depicts the structure of the 5′ flanking region of the human CDCA8 gene including the nucleotide sequence and putative regulatory elements (CDE-CHR) of the 5′ flanking region of the human CDCA8 gene. The first nucleotide of the known CDCA8 transcript is designated as +1. The putative binding elements for transcription factors are boxed. Part A (lower panel) depicts the alignment of the sequence with the consensus CDE and CHR sequences and with those of promoters from human AURKB, CCNDA, and CDC25.

Part B depicts the expression of CDCA8, AURKB, and E2F-1 in clinical samples of 14 NSCLC (T) and corresponding normal lung tissues (N), examined by semi-quantitative RT-PCR.

Part C depicts the promoter activity of 5′ flanking region of the human CDCA8 and AURKB gene enhanced by E2F-1.

FIG. 5 depicts the phosphorylation of CDCA8 by AURKB.

Part A depicts the dephosphorylation of endogenous CDCA8 protein in LC319 cells by treatment with lambda-phosphatase. The white arrow indicates phosphorylated CDCA8; black arrow, non-phosphorylated form.

Part B depicts the in vitro phosphorylation of recombinant CDCA8 (rhCDCA8) by recombinant ARUKB (rhAURKB).

Part C, (upper panels) depict the expression levels of endogenous AURKB and CDCA8 proteins, detected by western-blot analysis in LC319 cells transfected with siRNA against AURKB (si-AURKB: SEQ ID NO: 33). The expression levels of endogenous AURKB and CDCA8 transcripts, detected by semi-quantitative RT-PCR analysis in LC319 cells transfected with si-AURKB, are also shown. Part C (lower panels) depict the expression levels of endogenous AURKB and CDCA8 proteins, detected by western-blot analysis in LC319 cells transfected with si-AURKBs.siRNA oligos against AURKB (#1 and #2: SEQ ID NO: 59 and 60). The expression levels of endogenous AURKB and CDCA8 transcripts, detected by semi-quantitative RT-PCR analysis in LC319 cells transfected with siRNA oligos against AURKB (#1 and #2), are also shown.

FIG. 6 identifies the cognate phosphorylation sites on CDCA8 by AURKB.

Part A, (upper panel) depicts six full-length recombinant CDCA8 mutants that were substituted at putative serine/threonine phosphorylated sites to alanines; each construct contained two or three substitutions (CDCA8delta1, CDCA8delta2, CDCA8delta3, CDCA8delta4, CDCA8delta5, and CDCA8delta6). Part A (lower panel) depicts additional six full-length recombinant CDCA8 mutants that were substituted at either of six serine/threonine residues to an alanine residue (CDCA8delta7, CDCA8delta8, CDCA8delta9, CDCA8delta10, CDCA8delta11, and CDCA8delta12).

Part B depicts the results of in vitro kinase assays incubating wild-type and mutant CDCA8 proteins with recombinant AURKB. CDCA8delta2, -delta5, and -delta6 constructs resulted in a reduction of phosphorylation levels by AURKB (each of substituted residue was indicated as bold character on underline), whereas CDCA8delta1, CDCA8delta3, and CDCA8delta4 represented the same levels of phosphorylation compared to wild-type CDCA8.

As shown in Part C, CDCA8delta8, -delta9, -delta11, and -delta12 resulted in a reduction of phosphorylation, whereas CDCA8delta7 and CDCA8delta10 showed the same levels of phosphorylation compared with wild-type, indicating that CDCA8 was phosphorylated at Ser 154, Ser-219, Ser-275, and Thr-278 (indicated as bold character on underline) by AURKB.

Part D depicts the results of in vitro kinase assays incubating wild-type and mutant CDCA8 protein (CDCA8delta13), in which all of the four serine/threonines were substituted to an alanine, with recombinant AURKB. CDCA8delta13 construct resulted in complete diminishment of CDCA8 phosphorylation by AURKB.

FIG. 7 depicts the inhibition of growth of lung-cancer cells by cell-permeable CDCA8-peptides.

Part A depicts the reduction of the AURKB-dependent CDCA8-phosphorylation by cell-permeable CDCA8-peptides (11R-CDCA8₂₆₁₋₂₈₀), detected by in vitro kinase assay.

Part B, (upper panels) depict the expression levels of endogenous CDCA8 protein, detected by western-blot analysis of LC319 cells transfected with the 11R-CDCA8₂₆₁₋₂₈₀. Part B (lower panels) depict the expression levels of endogenous CDCA8 transcript, detected by semi-quantitative RT-PCR analysis of LC319 cells transfected with the 11R-CDCA8₂₆₁₋₂₈₀.

Part C, (upper panel) depicts the results of an MTT assay of LC319 cells, detecting a growth suppressive effect of 11R-CDCA8₂₆₁₋₂₈₀. Error bars represent the standard deviation of triplicate assays. Part C (lower panel) depicts the results of cell cycle analysis of LC319 cells after the treatment with 11R-CDCA8₂₆₁₋₂₈₀ peptides or Scramble peptides.

Part D, (upper panel) depicts the expression of CDCA8 protein in normal human lung fibroblasts derived MRC5 and CCD19-Lu cells compared with lung-cancer cell line LC319, examined by western-blot analysis. Part D (lower panel) depicts the results of an MTT assay, detecting no off-target effect of the 11R-CDCA8₂₆₁₋₂₈₀ peptides on MRC5 cells that scarcely expressed CDCA8 and AURKB protein.

Part E, (upper panel) depicts the expression of CDCA8 protein in human bronchial-epithelia-derived BEAS-2B cells compared with lung-cancer cell line LC319, examined by western-blot analysis. Part E (lower panel) depicts the results of an MTT assay; detecting no significant growth suppressive effect of the 11R-CDCA8₂₆₁₋₂₈₀ peptides on BEAS-2B cells.

### DETAILED DESCRIPTION OF THE PRESENT INVENTION

The present invention constitutes an advancement in the field of cancer therapy by providing screening and therapeutic methods that target the interaction between CDCA8 and AURKB. As discussed in detail herein, the co-activation of CDCA8 and AURKB, and their cognate interactions, play a significant role in lung-cancer progression. Accordingly, agents that directly or indirectly inhibit the formation of the CDCA8-AURKB complex, by inhibiting the expression of CDCA8 or AURK8 or both, by inhibiting the binding between CDCA8 and AURKB, or by inhibiting the phosphorylation of CDCA8 by AURK8, find utility in the treatment and/or prevention of cancer, more particularly non-small-cell lung cancer. In addition, expression levels of CDCA8 and/or AURKB may be correlated to a lung cancer prognosis.

Although any methods and materials similar or equivalent to those described herein can be used in the practice or testing of embodiments of the present invention, the preferred methods, devices, and materials are now described. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the present invention, the following definitions apply:

The words “a”, “an” and “the” as used herein mean “at least one” unless otherwise specifically indicated.

The term “efficacious” refers to a treatment that results in a decrease in size, prevalence or metastatic potential of NSCLC in a subject. When a treatment is applied prophylactically, “efficacious” means that the treatment retards or prevents the occurrence of NSCLC or alleviates a clinical symptom of NSCLC. The assessment of NSCLC can be made using standard clinical protocols. Furthermore, the efficaciousness of a treatment may be determined in association with any known method for diagnosing or treating NSCLC. For example, NSCLC is frequently diagnosed histopathologically or by identifying symptomatic anomalies such as chronic cough, hoarseness, coughing up blood, weight loss, loss of appetite, shortness of breath, wheezing, repeated bouts of bronchitis or pneumonia and chest pain.

Herein, the term “preventing” means that the agent is administered prophylactically to retard or suppress the forming of tumor or retards, suppresses, or alleviates at least one clinical symptom of cancer. Assessment of the state of tumor in a subject can be made using standard clinical protocols. Prophylactic administration may occur prior to the manifestation of overt clinical symptoms of disease, such that a disease or disorder is prevented or, alternatively, delayed in its progression. Thus, in the context of the present invention, “prevention” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications. Accordingly, the present invention encompasses a wide range of prophylactic therapies aimed at alleviating the severity of cancer, particularly NSCLC.

The terms “isolated” and “purified” used herein in relation to a substance (e.g., polypeptide, antibody, polynucleotide, etc.) indicate that the substance is substantially free from at least one substance that may else be included in the natural source. Thus, an isolated or purified antibody refers to antibodies that is substantially free of cellular material such as carbohydrate, lipid, or other contaminating proteins from the cell or tissue source from which the protein (antibody) is derived, or substantially free of chemical precursors or other chemicals when chemically synthesized.

The term “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, it is also preferably substantially free of culture medium, which includes preparations of polypeptide with culture medium less than about 20%, 10%, or 5% of the volume of the protein preparation. When the polypeptide is produced by chemical synthesis, it is preferably substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In a preferred embodiment, antibodies of the present invention are isolated or purified.

An “isolated” or “purified” nucleic acid molecule, such as a cDNA molecule, can be substantially free of other cellular material or culture medium when produced by recombinant techniques, or substantially free of chemical precursors or other chemicals when chemically synthesized. In a preferred embodiment, nucleic acid molecules encoding antibodies of the present invention are isolated or purified.

The terms “polypeptide”, “peptide” and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “polynucleotides”, “nucleotides”, “nucleic acids”, and “nucleic acid molecules” are used interchangeably unless otherwise specifically indicated and, similarly to the amino acids, are referred to by their commonly accepted single-letter codes. Similar to the amino acids, they encompass both naturally-occurring and non-naturally occurring nucleic acid polymers.

As use herein, the term “double-stranded molecule” refers to a nucleic acid molecule that inhibits expression of a target gene including, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As noted elsewhere herein, the present invention also contemplates functional equivalents of CDCA8 and AURKB. As used herein, a “functional equivalent” of a reference protein is a polypeptide that has a biological activity, in particular binding activity, equivalent to the reference protein. In the context of the present invention, the term “functionally equivalent of CDCA8” means that the subject protein can be phosphorylated by AURKB and includes phosphorylation sites. Whether or not a subject protein is the target for phosphorylation can be determined in accordance with the present invention. For example, kinase activity for CDCA8 can be determined by incubating a polypeptide under conditions suitable for phosphorylation of CDCA8 and detecting the phosphorylated CDCA8 level. For example, the known phosphorylation sites of CDCA8 by AURKB are the Ser-154, Ser-219, Ser-275, and Thr-278 residues. In a preferred embodiment, functional equivalent of CDCA8 may be phosphorylated by AURKB to promote cell proliferation. Activity to promote the cell proliferation can also be evaluated in accordance with the present invention. On the other hand, the term of “functionally equivalent of AURKB” means that the subject protein has the kinase activity, more preferably, the protein can phosphorylate the CDCA8 or functional equibalent thereof. In a preferred embodiment, functional equivalent of AURKB may phosphorylate CDCA8 to promote cell proliferation.

Accordingly, in the context of the present invention, the phrase “CDCA8 gene” encompasses polynucleotides that encode the CDCA8 protein or any of the functional equivalents of the CDCA8 protein. Similarly, the phrase “AURKB gene” encompasses polynucleotides that encode the AURKB protein or any of the functional equivalents of the AURKB protein.

As used herein, the term “antibody” refers to an immunoglobulin molecule having a specific structure, that interacts (i.e., binds) only with the antigen that was used for synthesizing the antibody or with an antigen closely related thereto. In the context of the present invention, an antibody may be a fragment of an antibody or a modified antibody, so long as it binds to the proteins encoded by the CDCA8 or AURKB genes.

In the context of the present invention, “inhibition of binding” between two proteins refers to at least reducing binding between the proteins. Thus, in some cases, the percentage of binding pairs in a sample will be decreased compared to an appropriate (e.g., not treated with test compound or from a non-cancer sample, or from a cancer sample) control. The reduction in the amount of proteins bound may be, e.g., less than 90%, 80%, 70%, 60%, 50%, 40%, 25%, 10%, 5%, 1% or less (e.g., 0%), than the pairs bound in a control sample.

### Screening for a Compound for Treating or Preventing NSCLC:

As described above, in the course of the present invention it was discovered that CDCA8 interacts with AURKB in NSCLC cells. Thus, the present invention provides methods of screening for a compound suitable for the treatment and/or prevention of NSCLC. Alternatively, a candidate compound suitable for the treatment and/or prevention of NSCLC may be identifyed by the present invention. Such methods include the steps of:
(a) contacting an AURKB polypeptide or functional equivalent thereof with a CDCA8 polypeptide or functional equivalent thereof in the presence of a test compound;
(b) assaying the binding between the polypeptides of step (a); and
(c) selecting the test compound that inhibits the binding between the AURKB and CDCA8 polypeptides.

In the context of the present invention, a functional equivalent of a CDCA8 or AURKB polypeptide is a polypeptide that has a biological activity equivalent to a CDCA8 polypeptide (SEQ ID NO: 2) or AURKB polypeptide (SEQ ID NO: 4), respectively.

As a method of screening for compounds that inhibit the phosphorylation of CDCA8 by AURKB, many methods well known to those skilled in the art can be used. For example, screening can be carried out using an in vitro assay system, such as a cellular system.

The present invention is also based on the discovery that AURKB has the kinase activity for CDCA8. For example, phosphorylation sites of CDCA8 by AURKB are the Ser-154, Ser-219, Ser-275, and Thr-278 residues. Accordingly, in one aspect, the present invention involves identifying test compounds that regulate AURKB-mediated phosphorylation of CDCA8. Accordingly, the present invention provides a method of screening for compounds suitable for the treatment and/or prevention of NSCLC. Alternatively, a candidate compound suitable for the treatment and/or prevention of NSCLC may be identifyed by the present invention. Such methods including the steps of:
(a) incubating CDCA8 and AURKB in the presence of a test compound under conditions suitable for the phosphorylation of CDCA8 by AURKB, wherein the CDCA8 is a polypeptide selected from the group consisting of
   i. a polypeptide the amino acid sequence of SEQ ID NO: 2 (CDCA8);
   ii. a polypeptide having the amino acid sequence of SEQ ID NO: 2 wherein one or more amino acids are substituted, deleted, or inserted, provided the polypeptide has a biological activity equivalent to the polypeptide consisting of the amino acid sequence of SEQ ID NO: 2;
   iii. a polypeptide encoded by a polynucleotide that hybridizes under stringent conditions to a polynucleotide consisting of the nucleotide sequence of SEQ ID NO: 1, provided the polypeptide has a biological activity equivalent to a polypeptide consisting of the amino acid sequence of SEQ ID NO: 2, and
wherein the AURKB is a polypeptide selected from the group consisting of:
   i. a polypeptide the amino acid sequence of SEQ ID NO: 4 (AURKB);
   ii. a polypeptide having the amino acid sequence of SEQ ID NO: 4 wherein one or more amino acids are substituted, deleted, or inserted, provided the polypeptide has a biological activity equivalent to the polypeptide consisting of the amino acid sequence of SEQ ID NO: 4;
   iii. a polypeptide encoded by a polynucleotide that hybridizes under stringent conditions to a polynucleotide consisting of the nucleotide sequence of SEQ ID NO: 3, provided the polypeptide has a biological activity equivalent to a polypeptide consisting of the amino acid sequence of SEQ ID NO: 4;
(b) detecting a phosphorylation level of the CDCA8;
(c) comparing the phosphorylation level of the CDCA8 to a control level; and
(d) selecting a compound that decreases the phosphorylation level of the CDCA8 as compared to the control level.

Herein, the method of screening for a compound suitable for treating and/or preventing non-small cell lung cancer (NSCLC) may include the step of detecting the phosphorylation level of the CDCA8 at one or more phosphorylation site selected from the group consisting of the Ser-154, Ser-219, Ser-275, and Thr-278 residues of the amino acid sequence of SEQ ID NO: 2, or homologous positions of the polypeptide.

In another aspect of the invention, a kit for screening for compounds suitable for the treatment and/or prevention NSCLC is also provided. The kit optionally includes the components of:
(a) a polypeptide selected from the group consisting of:
   i. a polypeptide having the amino acid sequence of SEQ ID NO: 2 (CDCA8);
   ii. a polypeptide having the amino acid sequence of SEQ ID NO: 2 wherein one or more amino acids are substituted, deleted, or inserted, provided the polypeptide has a biological activity equivalent to the polypeptide of the amino acid sequence of SEQ ID NO: 2; and
   iii. a polypeptide encoded by a polynucleotide that hybridizes under stringent conditions to a polynucleotide of the nucleotide sequence of SEQ ID NO: 1 provided the polypeptide has a biological activity equivalent to a polypeptide of the amino acid sequence of SEQ ID NO: 2 and
(b) a polypeptide selected from the group consisting of:
   i. a polypeptide having the amino acid sequence of SEQ ID NO: 4 (AURKB);
   ii. a polypeptide having the amino acid sequence of SEQ ID NO: 4 wherein one or more amino acids are substituted, deleted, or inserted, provided the polypeptide has a biological activity equivalent to the polypeptide of the amino acid sequence of SEQ ID NO: 4; and
   iii. a polypeptide encoded by a polynucleotide that hybridizes under stringent conditions to a polynucleotide of the nucleotide sequence of SEQ ID NO: 3, provided the polypeptide has a biological activity equivalent to a polypeptide of the amino acid sequence of SEQ ID NO: 4; and
(c) a reagent for detecting a phosphorylation level of CDCA8.

Further, this invention also provides a kit for screening for a compound suitable for the treatment and/or prevention NSCLC. The kit optionally includes the components of:
(a) a cell expressing a polypeptide selected from the group consisting of:
   i. a polypeptide having the amino acid sequence of SEQ ID NO: 2 (CDCA8);
   ii. a polypeptide having the amino acid sequence of SEQ ID NO: 2 wherein one or more amino acids are substituted, deleted, or inserted, provided the polypeptide has a biological activity equivalent to the polypeptide of the amino acid sequence of SEQ ID NO: 2;
   iii. a polypeptide encoded by a polynucleotide that hybridizes under stringent conditions to a polynucleotide of the nucleotide sequence of SEQ ID NO: 1, provided the polypeptide has a biological activity equivalent to a polypeptide of the amino acid sequence of SEQ ID NO: 2; and
(b) a reagent for detecting a phosphorylation level of CDCA8.

Furthermore, the kit for screening for compounds suitable for the treatment and/or prevention NSCLC may optionally include cells further expressing a polypeptide selected from the group consisting of:
i. a polypeptide having the amino acid sequence of SEQ ID NO: 4 (AURKB);
ii. a polypeptide having the amino acid sequence of SEQ ID NO: 4 wherein one or more amino acids are substituted, deleted, or inserted, provided the polypeptide has a biological activity equivalent to the polypeptide of the amino acid sequence of SEQ ID NO: 4; and
iii. a polypeptide encoded by a polynucleotide that hybridizes under stringent conditions to a polynucleotide of the nucleotide sequence of SEQ ID NO: 3, provided the polypeptide has a biological activity equivalent to a polypeptide of the amino acid sequence of SEQ ID NO: 4.

In another aspect, the cell used in the kit is NSCLC cells.

In the present invention, the kit may further include phosphate donor. The kit of the present invention may also include an antibody that recognizes phosphorylated Ser-275, Ser-219, Ser-275 and Thr-278 of CDCA8 as a reagent for detecting a phosphorylated CDCA8. Consequently, the present invention also provides the kit for screening for a compound suitable for the treatment and/or prevention NSCLC, wherein the reagent for detecting a phosphorylation level of CDCA8 is an antibody that recognizes the phosphorylation at any one of the phosphorylation sites selected from the group consisting of the Ser-154, Ser-219, Ser-275, and Thr-278 residues of the amino acid sequence of SEQ ID NO: 2. Further, the present invention also provide a composition for treating or preventing non-small cell lung cancer (NSCLC), the composition composed of a pharmaceutically effective amount of a compound that decreases a kinase activity of AURKB for CDCA8 in combination with a pharmaceutically acceptable carrier.

In the context of the present invention, the conditions suitable for the phosphorylation of CDCA8 by AURKB may be provided with an incubation of CDCA8 and AURKB in the presence of phosphate donor, e.g. ATP. The conditions suitable for the CDCA8 phosphorylation by AURKB also include culturing cells expressing the polypeptides. For example, the cell may be a transformant cell harboring an expression vector containing a polynucleotide that encodes the polypeptide. After the incubation, the phosphorylation level of the CDCA8 can be detected with an antibody recognizing phosphorylated CDCA8.

Prior to the detection of phosphorylated CDCA8, CDCA8 may be separated from other elements, or cell lysate of CDCA8 expressing cells. For instance, gel electrophoresis may be used for the separation of CDCA8 from remaining components. Alternatively, CDCA8 may be captured by contacting CDCA8 with a carrier having an anti-CDCA8 antibody. When the labeled phosphate donor is used, the phosphorylation level of the CDCA8 can be detected by tracing the label. For example, when radio-labeled ATP (e.g. ³²P-ATP) is used as a phosphate donor, radio activity of the separated CDCA8 correlates with the phosphorylation level of the CDCA8. Alternatively, an antibody specifically recognizing phosphorylated CDCA8 from unphosphorylated CDCA8 may be used to detect phosphorylated CDCA8. Preferably, the antibody recognizes phosphorylated CDCA8 at any of the Ser-154, Ser-219, Ser-275, and Thr-278 residues.

Furthermore, the present invention is also based on the discovery that phosphorylated CDCA8 by AURKB can avoid degradation. More specifically, it was confirmed that the amount of CDCA8 protein is dramatically decreased in cells of which expression level of AURKB protein is reduced by siRNA, while a level of CDCA8 transcripts in the same cells was not influenced by si-AURKB (FIG. 5C, lower panel). Accordingly, the amount of the CDCA8 polypeptide is preferable indicator for screening for compounds suitable for the treatment and/or prevention of NSCLC. Alternatively, a candidate compound suitable for the treatment and/or prevention of NSCLC may be identifyed by the present invention. Such methods including the steps of:
(a) incubating CDCA8 and AURKB in the presence of a test compound under conditions suitable for the degradation of unphosphorylated CDCA8, wherein the CDCA8 is a polypeptide selected from the group consisting of
   i. a polypeptide the amino acid sequence of SEQ ID NO: 2 (CDCA8);
   ii. a polypeptide having the amino acid sequence of SEQ ID NO: 2 wherein one or more amino acids are substituted, deleted, or inserted, provided the polypeptide has a biological activity equivalent to the polypeptide consisting of the amino acid sequence of SEQ ID NO: 2;
   iii. a polypeptide encoded by a polynucleotide that hybridizes under stringent conditions to a polynucleotide consisting of the nucleotide sequence of SEQ ID NO: 1, provided the polypeptide has a biological activity equivalent to a polypeptide consisting of the amino acid sequence of SEQ ID NO: 2;
, wherein the AURKB is a polypeptide selected from the group consisting of:
   i. a polypeptide the amino acid sequence of SEQ ID NO: 4 (AURKB);
   ii. a polypeptide having the amino acid sequence of SEQ ID NO: 4 wherein one or more amino acids are substituted, deleted, or inserted, provided the polypeptide has a biological activity equivalent to the polypeptide consisting of the amino acid sequence of SEQ ID NO: 4;
   iii. a polypeptide encoded by a polynucleotide that hybridizes under stringent conditions to a polynucleotide consisting of the nucleotide sequence of SEQ ID NO: 3, provided the polypeptide has a biological activity equivalent to a polypeptide consisting of the amino acid sequence of SEQ ID NO: 4;
(b) detecting the amount of the CDCA8;
(c) comparing the amount of the CDCA8 to a control amount such as in the absence of the test agent; and
(d) selecting a compound that decreases the amount of the CDCA8 as compared to the control.

In the context of the present invention, the conditions suitable for the degradation of unphosphorylated CDCA8 may be provided with an incubation of CDCA8 and AURKB in the presence of protease that specifically cleaves unphosphorylated CDCA8 protein. In the present invention, when CDCA8 acquired resistance to cleavage with a protease through the phosphorylation thereof, the protease specifically cleaves unphosphorylated CDCA8. Alternatively, a protease whose cleavage activity against CDCA8 is reduced by the phosphorylation of CDCA8 may be defined as unphosphorylated CDCA8 specific protease in the present invention. In a preffered embodyment, the cleavage activity of unphosphorylated CDCA8 specific protease is reduced into, for example 50% or less, preferably 60% or less, more preferably 70% or 80% or less by the phosphorylation, compare to that of unphosphorylated CDCA8. For example, ubiqitin-protease system component is preferable unphosphorylated CDCA8 specific protease.

In some preferred embodiments, CDCA8 and AURKB may be incubated with a test compound under the condition suitable for both of phoshorylation of CDCA8 by AURKB, and the degradation of unphosphorylated CDCA8. Such condition may be provided by culturing cells expressing the polypeptides or lysate thereof. For example, the cell may be a transformant cell harboring an expression vector containing a polynucleotide that encodes the polypeptides. After the incubation with a test compound, the amount of the CDCA8 can be detected with an antibody recognizing CDCA8. For instance, in the present invention, immunoassay or western-botting assay may be applied to detection of CDCA8.

In order to identifying the compound that interferes the phosphorylation of CDCA8 by AURKB specifically, further screening may be performed, prior to or after the above mentioned screening method. For example, by selecting a compound that binds to AURKB prior to or after the screening, candidate compound that inhibits the function of AURKB may be identifyed. Such compound may be selected by contacting a test compound with AURKB or fragment thereof, and identifying a compound that binds to the AURKB or fragment thereof. Alternatively, it may also be confirmed whether a test compound affects the expression level of CDCA8 by determining the amount of CDCA8 transcript.

Further, this invention also provides a kit for screening for a compound suitable for the treatment and/or prevention NSCLC. The kit optionally includes the components of:
(a) a cell expressing a polypeptide selected from the group consisting of:
   i. a polypeptide comprising the amino acid sequence of SEQ ID NO: 2 (CDCA8);
   ii. a polypeptide comprising the amino acid sequence of SEQ ID NO: 2 wherein one or more amino acids are substituted, deleted, or inserted, provided said polypeptide has a biological activity equivalent to the polypeptide consisting of the amino acid sequence of SEQ ID NO: 2;
   iii. a polypeptide encoded by a polynucleotide that hybridizes under stringent conditions to a polynucleotide consisting of the nucleotide sequence of SEQ ID NO: 1, provided the polypeptide has a biological activity equivalent to a polypeptide consisting of the amino acid sequence of SEQ ID NO: 2; and
(b) a reagent for detecting a level of CDCA8.

Furthermore, the kit for screening for compounds suitable for the treatment and/or prevention NSCLC may optionally include cells further expressing a polypeptide selected from the group consisting of:
i. a polypeptide comprising the amino acid sequence of SEQ ID NO: 4 (AURKB);
ii. a polypeptide comprising the amino acid sequence of SEQ ID NO: 4 wherein one or more amino acids are substituted, deleted, or inserted, provided said polypeptide has a biological activity equivalent to the polypeptide consisting of the amino acid sequence of SEQ ID NO: 4; and
iii. a polypeptide encoded by a polynucleotide that hybridizes under stringent conditions to a polynucleotide consisting of the nucleotide sequence of SEQ ID NO: 3, provided the polypeptide has a biological activity equivalent to a polypeptide consisting of the amino acid sequence of SEQ ID NO: 4.

Preferably, the cell expressing CDCA8 and AURKB or the functional equibalents thereof is NSCLC cell.

Prior to the detection of CDCA8, CDCA8 may be separated from other elements, or cell lysate of CDCA8 expressing cells. For instance, gel electrophoresis may be used for the separation of CDCA8 from remaining components. Alternatively, an antibody specifically recognizing CDCA8 may be used to detect CDCA8.

Methods for preparing polypeptides functionally equivalent to a given protein are well known by a person skilled in the art and include known methods of introducing mutations into the protein. Generally, it is known that modifications of one or more amino acid in a protein do not influence the function of the protein (Mark D F et al., Proc Natl Acad Sci USA 1984, 81: 5662-6; Zoller M J & Smith M, Nucleic Acids Res 1982, 10: 6487-500; Wang A et al., Science 1984, 224:1431-3; Dalbadie-McFarland G et al., Proc Natl Acad Sci USA 1982, 79: 6409-13). In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids, or those considered to be “conservative modifications”, wherein the alteration of a protein results in a protein with similar functions, are contemplated in the context of the instant invention.

For example, one skilled in the art can prepare polypeptides functionally equivalent to CDCA8 or AURKB by introducing an appropriate mutation in the amino acid sequence of either of these proteins using, for example, site-directed mutagenesis (Hashimoto-Gotoh et al., Gene 152:271-5 (1995); Zoller and Smith, Methods Enzymol 100: 468-500 (1983); Kramer et al., Nucleic Acids Res. 12:9441-56 (1984); Kramer and Fritz, Methods Enzymol 154: 350-67 (1987); Kunkel, Proc Natl Acad Sci USA 82: 488-92 (1985); Kunkel T A, et al., Methods Enzymol. 1991; 204:125-39). The polypeptides of the present invention includes those having the amino acid sequences of CDCA8 or AURKB in which one or more amino acids are mutated, provided the resulting mutated polypeptides are functionally equivalent to CDCA8 or AURKB, respectively. So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, typically 20 amino acids or less, more typically 10 amino acids or less, preferably 5-6 amino acids or less, and more preferably 1-3 amino acids.

The amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Note, the parenthetic letters indicate the one-letter codes of amino acids. Furthermore, conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins 1984).

Such conservatively modified polypeptides are included in the present CDCA8 or AURKB protein. However, the present invention is not restricted thereto and the CDCA8 and AURKB proteins include non-conservative modifications so long as the binding activity of the original proteins is retained. Furthermore, the modified proteins do not exclude polymorphic variants, interspecies homologues, and those encoded by alleles of these proteins.

An example of a polypeptide to which one or more amino acids residues are added to the amino acid sequence of CDCA8 or AURKB is a fusion protein containing CDCA8 or AURKB, respectively. Accordingly, fusion proteins, i.e., fusions of CDCA8 or AURKB and other peptides or proteins, are included in the present invention. Fusion proteins can be made by techniques well known to a person skilled in the art, such as by linking the DNA encoding CDCA8 or AURKB with DNA encoding other peptides or proteins, so that the frames match, inserting the fusion DNA into an expression vector and expressing it in a host. There is no restriction as to the peptides or proteins fused to the protein of the present invention.

Known peptides that can be used as peptides to be fused to the CDCA8 or AURKB proteins include, for example, FLAG (Hopp T P et al., Biotechnology 1988 6: 1204-10), 6×His containing six His (histidine) residues, 10×His, Influenza agglutinin (HA), human c-myc fragment, VSP-GP fragment, p18HIV fragment, T7-tag, HSV-tag, E-tag, SV40T antigen fragment, lck tag, alpha-tubulin fragment, B-tag, Protein C fragment, and the like. Examples of proteins that may be fused to a protein of the invention include GST (glutathione-S-transferase), Influenza agglutinin (HA), immunoglobulin constant region, beta-galactosidase, MBP (maltose-binding protein), and such.

Fusion proteins can be prepared by fusing commercially available DNA, encoding the fusion peptides or proteins discussed above, with the DNA encoding the CDCA8 or AURKB proteins and expressing the fused DNA prepared.

An alternative method known in the art to isolate functionally equivalent polypeptides involves, for example, hybridization techniques (Sambrook et al., Molecular Cloning 2nd ed. 9.47-9.58, Cold Spring Harbor Lab. Press (1989)). One skilled in the art can readily isolate a DNA having high homology with CDCA8 or AURKB (i.e., SEQ ID NOs: 1 and 3, respectively), and isolate polypeptides functionally equivalent to the CDCA8 or AURKB from the isolated DNA. The proteins of the present invention include those that are encoded by DNA that hybridize with a whole or part of the DNA sequence encoding CDCA8 or AURKB and are functionally equivalent to CDCA8 or AURKB. These polypeptides include mammalian homologues corresponding to the protein derived from humans (for example, a polypeptide encoded by a monkey, rat, rabbit and bovine gene). In isolating a cDNA highly homologous to the DNA encoding CDCA8 or AURKB from animals, it is particularly preferable to use lung cancer tissues.

The condition of hybridization for isolating a DNA encoding a protein functional equivalent to the human CDCA8 or AURKB protein can be routinely selected by a person skilled in the art. The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). In the context of the present invention, suitable hybridization conditions can be routinely selected by a person skilled in the art

Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is preferably at least two times of background, more preferably 10 times of background hybridization.

Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 50° C. Suitable hybridization conditions may also include prehybridization at 68° C. for 30 min or longer using “Rapid-hyb buffer” (Amersham. LIFE SCIENCE), adding a labeled probe, and warming at 68° C. for 1 h or longer.

The washing step can be conducted, for example, under conditions of low stringency. Thus, an exemplary low stringency condition may include, for example, 42° C., 2×SSC, 0.1% SDS, or preferably 50° C., 2×SSC, 0.1% SDS. Alternatively, an exemplary high stringency condition may include, for example, washing 3 times in 2×SSC, 0.01% SDS at room temperature for 20 min, then washing 3 times in 1×SSC, 0.1% SDS at 37° C. for 20 min, and washing twice in 1×SSC, 0.1% SDS at 50° C. for 20 min. However, several factors such as temperature and salt concentration can influence the stringency of hybridization and one skilled in the art can suitably select the factors to achieve the requisite stringency.

Preferably, the functionally equivalent polypeptide has an amino acid sequence with at least about 80% homology (also referred to as sequence identity) to the native CDCA8 or AURKB sequence disclosed here, more preferably at least about 85%, 90%, 95%, 96%, 97%, 98%, or 99% homology. The homology of a polypeptide can be determined by following the algorithm in “Wilbur and Lipman, Proc Natl Acad Sci USA 80: 726-30 (1983)”. In other embodiments, the functional equivalent polypeptide can be encoded by a polynucleotide that hybridizes under stringent conditions (as defined below) to a polynucleotide encoding such a functional equivalent polypeptide.

In place of hybridization, a gene amplification method, for example, the polymerase chain reaction (PCR) method, can be utilized to isolate a DNA encoding a polypeptide functionally equivalent to CDCA8 or AURKB, using a primer synthesized based on the sequence information for CDCA8 or AURKB.

A CDCA8 or AURKB functional equivalent useful in the context of the present invention may have variations in amino acid sequence, molecular weight, isoelectric point, the presence or absence of sugar chains, or form, depending on the cell or host used to produce it or the purification method utilized. Nevertheless, so long as it is a function equivalent of either the CDCA8 or AURKB polypeptide, it is within the scope of the present invention.

In some preferred embodiments, the functional equivalent of the CDCA8 polypeptide can include an amino acid sequence corresponding to the AURKB binding domain, for example the amino acid sequence of SEQ ID NO: 5 (NIKKLSNRLAQICSSIRTHK). Similarly, the functional equivalent of the AURKB polypeptide can include an amino acid sequence corresponding to the CDCA8 binding domain.

As discussed above, inhibition of binding between CDCA8 and AURKB leads to suppression of cell proliferation. Furthermore, inhibition of phosphorylation of CDCA8 by AURKB leads to suppression of cell proliferation. Accordingly, compounds that inhibit the binding or phosphorylation processes may serve as pharmaceuticals for treating or preventing NSCLCs.

The CDCA8 and AURKB polypeptides to be used for the screening methods of the present invention may be a recombinant polypeptide or a protein derived from the nature, or may also be a partial peptide thereof, so long as it retains the binding ability or phosphorylation activity of the full-length protein. Such partial peptides that retain the binding ability or phosphorylation activity are herein referred to as “functional equivalents”. The CDCA8 and AURKB polypeptides to be used in the screening methods can be, for example, a purified polypeptide, a soluble protein, a form bound to a carrier or a fusion protein fused with other polypeptides.

As a method of screening for compounds that inhibit the binding between CDCA8 and AURKB, many methods well known by one skilled in the art can be used. For example, screening can be carried out using an in vitro assay system, such as a cellular system. More specifically, first, either CDCA8 or AURKB may be bound to a support, and the other protein may be added together with a test compound thereto. Next, the mixture may be incubated, washed and the other protein bound to the support may be detected and/or measured.

Examples of supports that may be used for binding proteins include, for example, insoluble polysaccharides, such as agarose, cellulose and dextran; and synthetic resins, such as polyacrylamide, polystyrene and silicon; preferably commercial available beads and plates (e.g., multi-well plates, biosensor chip, etc.) prepared from the above materials may be used. When using beads, they may be filled into a column. Alternatively, the use of magnetic beads is also known in the art, and enables one to readily isolate proteins bound on the beads via magnetism.

The binding of a protein to a support may be conducted according to routine methods, such as chemical bonding and physical adsorption, for example. Alternatively, a protein may be bound to a support via antibodies that specifically recognize the protein. Moreover, binding of a protein to a support can be also conducted by means of avidin and biotin.

The binding between proteins is preferably carried out in buffer, examples of which include, but are not limited to, phosphate buffer and Tris buffer. However, the selected buffer must not inhibit binding between the proteins.

In the context of the present invention, a biosensor using the surface plasmon resonance phenomenon may be used as a mean for detecting or quantifying the bound protein. When such a biosensor is used, the interaction between the proteins can be observed in real-time as a surface plasmon resonance signal, using only a minute amount of polypeptide and without labeling (for example, BIAcore, Pharmacia). Therefore, it is possible to evaluate binding between the CDCA8 and AURKB using a biosensor such as BIAcore.

Alternatively, either CDCA8 or AURKB may be labeled, and the label of the bound protein may be used to detect or measure the bound protein. Specifically, after pre-labeling one of the proteins, the labeled protein may be contacted with the other protein in the presence of a test compound, and then bound proteins may be detected or measured according to the label after washing.

Labeling substances suitable for use in the context of the present invention include, but are not limited to, radioisotopes (e.g., ³H, ¹⁴C, ³²P, ³⁵S, ¹²⁵I, ¹³¹I), enzymes (e.g., alkaline phosphatase, horseradish peroxidase, beta-galactosidase, beta-glucosidase), fluorescent substances (e.g., fluorescein isothiosyanete (FITC), rhodamine) and biotin/avidin. When the protein is labeled with a radioisotope, the detection or measurement can be carried out by liquid scintillation. Alternatively, proteins labeled with enzymes can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Furthermore, binding of CDCA8 and AURKB can be also detected or measured using antibodies to CDCA8 or AURKB. For example, after contacting the CDCA8 polypeptide immobilized on a support with a test compound and AURKB, the mixture is incubated and washed, and detection or measurement can be conducted using an antibody against AURKB. Alternatively, AURKB may be immobilized on a support, and an antibody against CDCA8 may be used as the antibody.

When using an antibody in the context of a screening method of the present invention, the antibody is preferably labeled with one of the labeling substances mentioned above, and detected or measured based on the labeling substance. Alternatively, an antibody against CDCA8 or AURKB may be used as a primary antibody to be detected with a secondary antibody that is labeled with a labeling substance. Furthermore, an antibody bound to the protein in the screening of the present invention may be detected or measured using protein G or protein A column.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”).

In the two-hybrid system, for example, a CDCA8 polypeptide is fused to an SRF-binding region or GAL4-binding region and expressed in yeast cells. An AURKB polypeptide that binds to the CDCA8 polypeptide is fused to a VP16 or GAL4 transcriptional activation region and also expressed in the yeast cells in the existence of a test compound. Alternatively, an AURKB polypeptide may be fused to an SRF-binding region or GAL4-binding region, and a CDCA8 polypeptide fused to a VP16 or GAL4 transcriptional activation region. When the test compound does not inhibit the binding between CDCA8 and AURKB, the binding of the two activates a reporter gene, making positive clones detectable. As a reporter gene, in addition to the HIS3 gene, suitable examples include, but are not limited to, Ade2 gene, lacZ gene, CAT gene, luciferase gene and the like.

Alternatively, the screening method of the present invention may include a reporter assay system. The reporter construct required for such a screening method can be prepared by introducing the transcriptional regulatory region of CDCA8 or AURKB gene and a reporter gene into a vector. The vector may be then introduced into a host cell and the expression level or activity of the reporter gene may be measured as compared to the control, under the influence of various test compounds. Suitable reporter genes and host cells are well known in the art.

The transcriptional regulatory region may be, for example, the promoter sequence of the CDCA8 or AURKB gene. The reporter construct required for the screening can be prepared by connecting reporter gene sequence to the transcriptional regulatory region of CDCA8 or AURKB gene. The transcriptional regulatory region of CDCA8 or AURKB gene herein is the region from start codon to at least 500 bp upstream, preferably 1000 bp, more preferably 5000 or 10000 bp upstream. A nucleotide segment containing the transcriptional regulatory region can be isolated from a genome library or can be propagated by PCR. Methods for identifying a transcriptional regulatory region, and also assay protocol are well known (Molecular Cloning third edition chapter 17, 2001, Cold Springs Harbor Laboratory Press).

Moreover, the sequence of the transcriptional regulatory regions of the CDCA8 or AURKB genes, respectively, are known to those skilled in the art. For example, nucleotide sequence selected from the 5′ flanking region of the CDCA8 or AURKB gene, and including the E2F-1 binding motif may be used as the transcriptional regulatory region. In the context of the present invention, a polynucleotide sequence such as that of SEQ ID NO: 56 can be used as an AURKB promoter sequence while a polynucleotide sequence such as that of SEQ ID NO: 57 can be used as a CDCA8 promoter sequence. In fact, polynucleotide sequences consisting of the nucleotide sequence of SEQ ID NO: 56 and 57 constitute preferred promoter sequences for AURKB and CDCA8, respectively. In the context of the present invention, for example, a reporter construct can be prepared by replacing the promoter regions of known reporter constructs with those of the AURKB or CDCA8 genes.

Any test compound, for example, cell extracts, cell culture supernatant, products of fermenting microorganism, extracts from marine organism, plant extracts, purified or crude proteins, peptides, non-peptide compounds, synthetic micromolecular compounds and natural compounds, can be used in the context of the screening methods of the present invention. The test compound of the present invention can be also obtained using any of the numerous approaches in combinatorial library methods known in the art, including, but not limited to,

(1) biological libraries,

(2) spatially addressable parallel solid phase or solution phase libraries,

(3) synthetic library methods requiring deconvolution,

(4) the “one-bead one-compound” library method and

(5) synthetic library methods using affinity chromatography selection.

The biological library methods using affinity chromatography selection is limited to peptide libraries, while the other four approaches are applicable to peptide, non-peptide oligomer or small molecule libraries of compounds (Lam, Anticancer Drug Des. 12: 145-67 (1997)). Examples of methods for the synthesis of molecular libraries can be found in the art (DeWitt et al., Proc Natl Acad Sci USA. 1993 Aug. 1; 90(15):6909-13; Erb et al., Proc. Natl. Acad. Sci. USA 91: 11422-6 (1994); Zuckermann et al., J. Med. Chem. 37: 2678-85 (1994); Cho et al., Science 261: 1303-5 (1993); Carell et al., Angew. Chem. Int. Ed Engl. 33: 2059 (1994); Carell et al., Angew. Chem. Int. Ed. Engl. 33: 2061 (1994); Gallop et al., J. Med. Chem. 37: 1233-51 (1994)).

Libraries of compounds may be presented in solution (see Houghten, Bio/Techniques 13: 412-21 (1992)) or on beads (Lam, Nature 354: 82-4 (1991)), chips (Fodor, Nature 364: 555-6 (1993)), bacteria (U.S. Pat. No. 5,223,409), spores (U.S. Pat. Nos. 5,571,698; 5,403,484, and 5,223,409), plasmids (Cull et al., Proc. Natl. Acad. Sci. USA 89: 1865-9 (1992)) or phage (Scott and Smith, Science 249: 386-90 (1990); Devlin, Science 249: 404-6 (1990); Cwirla et al., Proc. Natl. Acad. Sci. USA 87: 6378-82 (1990); Felici, J. Mol. Biol. 222: 301-10 (1991); US Pat. Application 20020103360). The test compound exposed to a cell or protein according to the screening methods of the present invention may be a single compound or a combination of compounds. When a combination of compounds is used in the screening methods of the invention, the compounds may be contacted sequentially or simultaneously.

A compound isolated by the screening methods of the present invention is a candidate for drugs which inhibit the activity of CDCA8 and AURKB, such drugs being suited to the treatment and/or prevention of diseases attributed to, for example, cell proliferative diseases, such as NSCLC. For example, a compound isolated by the screening methods of the present invention may have at least one function selected from the group consisting of

i. inhibiting a binding between CDCA8 and AURKB;

ii. inhibiting a phosphorylation of CDCA8 by AURKB;

iii. inhibiting a transcription of either of CDCA8 and AURKB genes, or both; and

iv. inhibiting CDCA8 stabilization by AURKB,

and such compound can be used for treating or preventing diseases attributed to, for example, cell proliferative diseases, such as NSCLC

A compound in which a part of the structure of the compound obtained by the present screening methods of the present invention is converted by addition, deletion and/or replacement, is included in the compounds obtained by the screening methods of the present invention. A compound effective in suppressing the expression of over-expressed genes, i.e., the CDCA8 and AURKB genes, is deemed to have a clinical benefit and can be further tested for its ability to reduce or prevent cancer cell growth in animal models or test subjects.

The present invention may also include screening for proteins that bind to a CDCA8 or AURKB polypeptide to inhibit the interaction therebetween. To that end, many methods well known to those skilled in the art can be used. Such a screening can be conducted by, for example, an immunoprecipitation assay using methods well known in the art.

The proteins of the invention can be recombinantly produced using standard procedures. For example, a gene encoding a polypeptide of interest may be expressed in animal cells by inserting the gene into an expression vector for foreign genes, such as pSV2neo, pcDNA I, pcDNA3.1, pCAGGS and pCD8. The promoter to be used for the expression may be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193-9 (1991)), the RSV LTR promoter (Cullen, Methods in Enzymology 152: 684-704 (1987)) the SR alpha promoter (Takebe et al., Mol Cell Biol 8: 466-72 (1988)), the CMV immediate early promoter (Seed and Aruffo, Proc Natl Acad Sci USA 84: 3365-9 (1987)), the SV40 late promoter (Gheysen and Fiers, J Mol Appl Genet 1: 385-94 (1982)), the Adenovirus late promoter (Kaufman et al., Mol Cell Biol 9: 946-58 (1989)), the HSV TK promoter and so on.

The introduction of the gene into animal cells to express a foreign gene can be performed according to any conventional method, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B, Cell 76: 1025-37 (1994); Lamb et al., Nature Genetics 5: 22-30 (1993): Rabindran et al., Science 259: 230-4 (1993)), and so on.

The polypeptide can also be expressed as a fusion protein, including a recognition site (epitope) of a monoclonal antibody by introducing the epitope of the monoclonal antibody, whose specificity has been revealed, to the N- or C-terminus of the polypeptide. A commercially available epitope-antibody system can also be used (Experimental Medicine 13: 85-90 (1995)). Vectors which can express a fusion protein with, for example, beta-galactosidase, maltose binding protein, glutathione S-transferase, green florescence protein (GFP), and so on, by the use of its multiple cloning sites are commercially available.

As noted above, a fusion protein, prepared by introducing only small epitopes composed of several to a dozen amino acids so as not to change the property of the original polypeptide by the fusion, is also provided herein. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the CDCA8 or AURKB polypeptide (Experimental Medicine 13: 85-90 (1995)).

In immunoprecipitation, an immune complex is formed by adding these antibodies to cell lysate prepared using an appropriate detergent. The immune complex may be composed of the CDCA8 or AURKB polypeptide, a polypeptide having binding affinity for the CDCA8 or AURKB polypeptide, and an antibody. Immunoprecipitation can be also conducted using antibodies against the CDCA8 or AURKB polypeptide, in addition to antibodies against the above epitopes, which antibodies can be prepared according to conventional methods and may be in any form, such as monoclonal or polyclonal antibodies, and include, for example, antiserum obtained by immunizing an animal such as a rabbit with the polypeptide, all classes of polyclonal and monoclonal antibodies, as well as recombinant antibodies (e.g., humanized antibodies).

Specifically, antibodies against the CDCA8 or AURKB polypeptide can be prepared using techniques well known in the art. For example, the CDCA8 or AURKB polypeptides used as an antigen to obtain an antibody may be derived from any animal species, though it is preferably derived from a mammal such as a human, mouse, rabbit, or rat, more preferably from a human. The polypeptide used as the antigen can be recombinantly produced or isolated from natural sources. In the context of the present invention, the polypeptide to be used as an immunization antigen may be a complete protein or a partial peptide of the CDCA8 or AURKB polypeptide.

Any mammalian animal may be immunized with the antigen; however, the compatibility with parental cells used for cell fusion is preferably taken into account. In general, animals of the order Rodentia, Lagomorpha or Primate are used. Animals of the Rodentia order include, for example, mice, rats and hamsters. Animals of Lagomorpha order include, for example, hares, pikas, and rabbits. Animals of Primate order include, for example, monkeys of Catarrhini (old world monkey) such as Macaca fascicularis, rhesus monkeys, sacred baboons and chimpanzees.

Methods for immunizing animals with antigens are well known in the art. Intraperitoneal injection or subcutaneous injection of antigens is a standard method for immunizing mammals. More specifically, antigens may be diluted and suspended in an appropriate amount of phosphate buffered saline (PBS), physiological saline, etc. If desired, the antigen suspension may be mixed with an appropriate amount of a standard adjuvant, such as Freund's complete adjuvant, made into emulsion, and then administered to mammalian animals. Preferably, it is followed by several administrations of the antigen mixed with an appropriately amount of Freund's incomplete adjuvant every 4 to 21 days. An appropriate carrier may also be used for immunization. After immunization as above, the serum is examined by a standard method for an increase in the amount of desired antibodies.

Polyclonal antibodies against a CDCA8 or AURKB polypeptide may be prepared by collecting blood from the immunized mammal examined for the increase of desired antibodies in the serum, and by separating serum from the blood by any conventional method. Polyclonal antibodies include serum containing the polyclonal antibodies, as well as the fraction containing the polyclonal antibodies isolated from the serum. Immunoglobulin G or M can be prepared from a fraction which recognizes only the CDCA8 or AURKB polypeptide using, for example, an affinity column coupled with the polypeptide, and further purifying this fraction using protein A or protein G column.

To prepare monoclonal antibodies, immune cells are collected from the mammal immunized with the antigen and checked for the increased level of desired antibodies in the serum as described above, and are subjected to cell fusion. The immune cells used for cell fusion are preferably obtained from spleen. Other preferred parental cells to be fused with the above immunocyte include, for example, myeloma cells of mammalians, and more preferably myeloma cells having an acquired property for the selection of fused cells by drugs.

The above immunocyte and myeloma cells can be fused according to known methods, for example, the method of Milstein et al., (Galfre and Milstein, Methods Enzymol 73: 3-46 (1981)).

Resulting hybridomas obtained by the cell fusion may be selected by cultivating them in a standard selection medium, such as HAT medium (hypoxanthine; aminopterin, and thymidine containing medium). The cell culture is typically continued in the HAT medium for several days to several weeks, the time being sufficient to allow all the other cells, with the exception of the desired hybridoma (non-fused cells), to die. Then, the standard limiting dilution is performed to screen and clone a hybridoma cell producing the desired antibody.

In addition to the above method, in which a non-human animal is immunized with an antigen for preparing hybridoma, human lymphocytes, such as those infected by the EB virus, may be immunized with a CDCA8 or AURKB polypeptide, cells expressing such a polypeptide, or their lysates in vitro. Then, the immunized lymphocytes are fused with human-derived myeloma cells that are capable of indefinitely dividing, such as U266, to yield a hybridoma producing a desired human antibody that is able to bind to the CDCA8 or AURKB polypeptide (Unexamined Published Japanese Patent Application No. (JP-A) Sho 63-17688).

The obtained hybridomas may be subsequently transplanted into the abdominal cavity of a mouse and the ascites may be extracted. The obtained monoclonal antibodies can be purified by, for example, ammonium sulfate precipitation, a protein A or protein G column, DEAE ion exchange chromatography, or an affinity column to which any of the target proteins of the present invention (CDCA8 or AURKB polypeptide) is coupled. The antibody can be used not only in the present screening method, but also for the purification and detection of a CDCA8 or AURKB polypeptide. They may further serve as candidates for agonists and antagonists of the polypeptides of interest. In addition, such antibodies, serving as candidates for antagonists, can be applied to the antibody treatment for diseases related to the CDCA8 or AURKB polypeptide, including NSCLC as described infra.

Monoclonal antibodies thus obtained can be also recombinantly prepared using genetic engineering techniques (see, for example, Borrebaeck and Larrick, Therapeutic Monoclonal Antibodies, published in the United Kingdom by MacMillan Publishers LTD (1990)). For example, a DNA encoding an antibody may be cloned from an immune cell, such as a hybridoma or an immunized lymphocyte producing the antibody, inserted into an appropriate vector, and introduced into host cells to prepare a recombinant antibody. Such recombinant antibody can also be used in the context of the present screening.

Furthermore, an antibody used in the screening and so on may be a fragment of an antibody or a modified antibody, so long as it binds to one or both of CDCA8 and AURKB. For instance, the antibody fragment may be an Fab, F(ab′)₂, Fv, or single chain Fv (scFv), in which Fv fragments from H and L chains are ligated by an appropriate linker (Huston et al., Proc Natl Acad Sci USA 85: 5879-83 (1988)). More specifically, an antibody fragment may be generated by treating an antibody with an enzyme, such as papain or pepsin. Alternatively, a gene encoding an antibody fragment may be constructed, inserted into an expression vector, and expressed in an appropriate host cell (see, for example, Co et al., J Immunol 152: 2968-76 (1994); Better and Horwitz, Methods Enzymol 178: 476-96 (1989); Pluckthun and Skerra, Methods Enzymol 178: 497-515 (1989); Lamoyi, Methods Enzymol 121: 652-63 (1986); Rousseau et al., Methods Enzymol 121: 663-9 (1986); Bird and Walker, Trends Biotechnol 9: 132-7 (1991)).

An antibody may be modified by conjugation with a variety of molecules, such as polyethylene glycol (PEG). Modified antibodies can be obtained through chemically modification of an antibody. These modification methods are conventional in the field.

Alternatively, an antibody may be obtained as a chimeric antibody, between a variable region derived from a nonhuman antibody and a constant region derived from a human antibody, or as a humanized antibody, composed of a complementarity determining region (CDR) derived from a nonhuman antibody, a frame work region (FR) derived, from a human antibody, and a constant region. Such antibodies can be prepared using known technology.

Humanization can be performed by substituting rodent CDRs or CDR sequences for the corresponding sequences of a human antibody (see, e.g., Verhoeyen et al., Science 239:1534-6 (1988)). Accordingly, such humanized antibodies are chimeric antibodies, wherein substantially less than an intact human variable domain has been substituted by the corresponding sequence from a non-human species. Fully human antibodies composed of human variable regions in addition to human framework and constant regions can also be used. Such antibodies can be produced using various techniques known in the art. For example, in vitro methods involve use of recombinant libraries of human antibody fragments displayed on bacteriophage (e.g., Hoogenboom & Winter, J. Mol. Biol. 227:381-8 (1992). Similarly, human antibodies can be made by introducing of human immunoglobulin loci into transgenic animals, e.g., mice in which the endogenous immunoglobulin genes have been partially or completely inactivated. This approach is described, e.g., in U.S. Pat. Nos. 6,150,584, 5,545,807; 5,545,806; 5,569,825; 5,625,126; 5,633,425; 5,661,016.

Antibodies obtained as above may be purified to homogeneity. For example, the separation and purification of the antibody can be performed according to separation and purification methods used for general proteins. For example, the antibody may be separated and isolated by appropriately selected and combined column chromatographies, such as affinity chromatography, filter, ultrafiltration, salting-out, dialysis, SDS polyacrylamide gel electrophoresis, isoelectric focusing, and others (Antibodies: A Laboratory Manual. Ed Harlow and David Lane, Cold Spring Harbor Laboratory (1988)); however, the present invention is not limited thereto. A protein A column and protein G column can be used as the affinity column. Exemplary protein A columns to be used include, for example, Hyper D, POROS, and Sepharose F.F. (Pharmacia).

Exemplary chromatography, with the exception of affinity, includes, for example, ion-exchange chromatography, hydrophobic chromatography, gel filtration, reverse-phase chromatography, adsorption chromatography, and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

An immune complex can be precipitated, for example with Protein A sepharose or Protein G sepharose when the antibody is a mouse IgG antibody. If the CDCA8 or AURKB polypeptide is prepared as a fusion protein with an epitope, such as GST, an immune complex can be formed in the same manner as in the use of the antibody against the CDCA8 or AURKB polypeptide, using a substance specifically binding to these epitopes, such as glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the CDCA8 or AURKB polypeptide is difficult to detect with conventional staining methods, such as Coomassie staining or silver staining, the detection sensitivity for the protein can be improved by culturing cells in culture medium containing radioactive isotope, ³⁵S-methionine or ³⁵S-cystein, labeling proteins in the cells, and detecting the proteins. The target protein can be purified directly from the SDS-polyacrylamide gel and its sequence can be determined, when the molecular weight of the protein has been revealed.

A compound binding to the CDCA8 or AURKB polypeptide can also be screened using affinity chromatography. For example, a CDCA8 or AURKB polypeptide may be immobilized on a carrier of an affinity column, and a test compound is applied to the column. A test compound herein may be, for example, cell extracts, cell lysates, etc. After loading the test compound, the column is washed, and compounds bound to the CDCA8 or AURKB polypeptide can be prepared.

When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as a mean for detecting or quantifying the bound compound in the present invention. When such a biosensor is used, the interaction between the CDCA8 or AURKB polypeptide and a test compound can be observed in real-time as a surface plasmon resonance signal, using only a minute amount of polypeptide and without labeling (for example, BIAcore, Pharmacia). Therefore, it is possible to evaluate the binding between a CDCA8 or AURKB polypeptide and a test compound using a biosensor such as BIAcore.

The methods of screening for molecules that bind when an immobilized CDCA8 or AURKB polypeptide is exposed to synthetic chemical compounds, or natural substance banks or a random phage peptide display library, and the methods of screening using high-throughput based on combinatorial chemistry techniques (Wrighton et al., Science 273: 458-64 (1996); Verdine, Nature 384: 11-3 (1996)) to isolate not only proteins but chemical compounds that bind to a CDCA8 or AURKB protein (including agonist and antagonist) are well known to one skilled in the art.

Although the construction of test agent libraries is well known in the art, herein below, additional guidance in identifying test agents and construction libraries of such agents for the present screening methods are provided.

(i) Molecular Modeling

Construction of test agent libraries is facilitated by knowledge of the molecular structure of compounds known to have the properties sought, and/or the molecular structure of the target molecules to be inhibited, i.e., CDCA8 and AURKB. One approach to preliminary screening of test agents suitable for further evaluation is computer modeling of the interaction between the test agent and its target. In the present invention, modeling the interaction between CDCA8 and AURKB provides insight into both the details of the interaction itself, and suggests possible strategies for disrupting the interaction, including potential molecular inhibitors of the interaction.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., J Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once a putative inhibitor of the interaction between CDCA8 and AURKB has been identified, combinatorial chemistry techniques can be employed to construct any number of variants based on the chemical structure of the identified putative inhibitor, as detailed below. The resulting library of putative inhibitors, or “test agents” may be screened using the methods of the present invention to identify test agents of the library that disrupt the association between CDCA8 and AURKB.

(ii) Combinatorial Chemical Synthesis

Combinatorial libraries of test agents may be produced as part of a rational drug design program involving knowledge of core structures existing in known inhibitors of the interaction between CDCA8 and AURKB. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of combinatorial chemical libraries is well known to those of skill in the art, and may be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used Such chemistries include, but are not limited to:

peptides (e.g., PCT Publication No. WO 91/19735),

encoded peptides (e.g., WO 93/20242),

random bio-oligomers (e.g., WO 92/00091),

benzodiazepines (e.g., U.S. Pat. No. 5,288,514),

diversomers such as hydantoins, benzodiazepines and dipeptides (DeWitt et al., Proc Natl Acad Sci USA 1993, 90:6909-13),

vinylogous polypeptides (Hagihara et al., J Amer Chem Soc 1992, 114: 6568),

nonpeptidal peptidomimetics with glucose scaffolding (Hirschmann et al., J Amer Chem Soc 1992, 114: 9217-8),

analogous organic syntheses of small compound libraries (Chen et al., J. Amer Chem Soc 1994, 116: 2661),

oligocarbamates (Cho et al., Science 1993, 261: 1303), and/or

peptidylphosphonates (Campbell et al., J Org Chem 1994, 59: 658),

nucleic acid libraries (see Ausubel, Current Protocols in Molecular Biology 1995 supplement; Sambrook et al., Molecular Cloning: A Laboratory Manual, 1989, Cold Spring Harbor Laboratory, New York, USA),

peptide nucleic acid libraries (see, e.g., U.S. Pat. No. 5,539,083),

antibody libraries (see, e.g., Vaughan et al., Nature Biotechnology 1996, 14(3):309-14 and PCT/US96/10287),

carbohydrate libraries (see, e.g., Liang et al., Science 1996, 274: 1520-22; U.S. Pat. No. 5,593,853), and

small organic molecule libraries (see, e.g., benzodiazepines, Gordon E M. Curr Opin Biotechnol. 1995 Dec. 1; 6(6):624-31; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

(iii) Phage Display

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 10⁶-10⁸ chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Differences in the genetic makeup of individuals can result in differences in their relative abilities to metabolize various drugs. A compound that is metabolized in a subject to act as an anti-NSCLC agent can manifest itself by inducing a change in a gene expression pattern in the subject's cells from that characteristic of a cancerous state to a gene expression pattern characteristic of a non-cancerous state. Accordingly, the differentially expressed CDCA8 or AURKB genes disclosed herein allow for the selection of a putative therapeutic or prophylactic inhibitor of NSCLC specifically adequate for a subject by testing candidate compounds in a test cell (or test cell population) derived from the selected subject.

To identify an anti-NSCLC agent that is appropriate for a specific subject, a test cell or test cell population derived from the subject is exposed to a therapeutic agent and the expression of one or more of the CDCA8 or AURKB genes is determined.

The test cell is or the test cell population contains an NSCLC cell expressing a CDCA8 or AURKB gene. Preferably, the test cell or the test cell population includes a lung cell. For example, the test cell or test cell population may be incubated in the presence of a candidate agent and the pattern of gene expression of the test cell or cell population may be measured and compared to one or more reference profiles, e.g., an NSCLC reference expression profile or a non-NSCLC reference expression profile.

A decrease in the expression of CDCA8 or AURKB in a test cell or test cell population relative to a reference cell population containing NSCLC is indicative that the agent is therapeutically efficacious.

### Methods for Treating or Preventing NSCLC:

The present invention further provides a method for treating, alleviating and/or preventing NSCLC in a subject. Therapeutic compounds may be administered prophylactically or therapeutically to subjects suffering from or at risk of (or susceptible to) developing NSCLC. Such subjects may be identified using standard clinical methods or by detecting an aberrant level of expression or activity of CDCA8 or AURKB gene or polypeptide. Prophylactic administration typically occurs prior to the manifestation of overt clinical symptoms of disease, such that a disease or disorder is prevented or alternatively delayed in its progression.

The inventive method preferably results in a decrease in the expression or function, or both, of one or more gene products of genes whose expression is aberrantly increased in an NSCLC cell relative to normal cells of the same tissue type from which the NSCLC cells are derived. The expression may be inhibited by any method known in the art. For example, a subject may be treated with an effective amount of a compound that decreases the amount of a CDCA8 or AURKB gene in the subject. Administration of the compound can be systemic or local. Such therapeutic compounds include compounds that decrease the expression level of such gene that endogenously exists in the NSCLC cells (i.e., compounds that down-regulate the expression of CDCA8 or AURKB genes). The administration of such therapeutic compounds counter the effects of aberrantly-over expressed gene(s) in the subjects NSCLC cells and are expected to improve the clinical condition of the subject. Such compounds can be obtained by the screening method of the present invention described above.

Alternatively, the expression of CDCA8 or AURKB can be inhibited by administering to the subject a nucleic acid that inhibits or antagonizes the expression of the over-expressed gene(s). Antisense oligonucleotides, siRNAs or ribozymes which disrupt the expression of the over-expressed gene(s) can be used for inhibiting the expression of the over-expressed gene(s).

As noted above, antisense-oligonucleotides corresponding to any of the nucleotide sequence of a CDCA8 or AURKB gene can be used to reduce the expression level of the gene. Antisense-oligonucleotides corresponding to the CDCA8 or AURKB genes that are up-regulated in NSCLC are useful in the treatment or prevention of NSCLC. Specifically, antisense-oligonucleotides against the genes may act by binding to any of the corresponding polypeptides encoded by these genes, or mRNAs corresponding thereto, thereby inhibiting the transcription or translation of the genes, promoting the degradation of the mRNAs, and/or inhibiting the expression of proteins encoded by the CDCA8 or AURKB nucleotides, and finally inhibiting the function of the proteins. The term “antisense-oligonucleotides” as used herein encompasses both nucleotides that are entirely complementary to the target sequence and those having a mismatch of one or more nucleotides, so long as the antisense-oligonucleotides can specifically hybridize to the target sequence. For example, the antisense-oligonucleotides of the present invention include polynucleotides having a homology (also referred to as sequence identity) of at least 70% or higher, preferably at 80% or higher, more preferably 90% or higher, even more preferably 95% or higher over a span of at least 15 continuous nucleotides up to the full length sequence of any of the nucleotide sequences of a CDCA8 or AURKB gene. Algorithms known in the art can be used to determine the homology. Furthermore, derivatives or modified products of the antisense-oligonucleotides can also be used as antisense-oligonucleotides in the present invention. Examples of such modified products include lower alkyl phosphonate modifications, such as methyl-phosphonate-type or ethyl-phosphonate-type, phosphorothioate modifications and phosphoroamidate modifications

siRNA molecules of the invention can also be defined by their ability to hybridize specifically to mRNA or cDNA from the genes disclosed here. In the context of the present invention, the terms “hybridize” and “hybridize specifically” are used interchangeably to refer the ability of two nucleic acid molecules to hybridize under “stringent hybridization conditions”. The phrase “stringent hybridization conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993).

Generally, stringent conditions are selected to be about 5-10 degrees C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions include the following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C. The antisense-oligonucleotides and derivatives thereof act on cells producing the proteins encoded by a CDCA8 or AURKB gene by binding to the DNA or mRNA encoding the protein, inhibiting transcription or translation thereof, promoting the degradation of the mRNAs and inhibiting the expression of the protein, thereby resulting in the inhibition of the protein function.

Antisense-oligonucleotides and derivatives thereof can be made into an external preparation, such as a liniment or a poultice, by mixing with a suitable base material which is inactive against the derivative.

The antisense-oligonucleotides of the invention inhibit the expression of at least one protein encoded by a CDCA8 or AURKB gene, and thus are useful for suppressing the biological activity of the respective protein.

The polynucleotides that inhibit one or more gene products of over-expressed genes also include small interfering RNAs (siRNA) composed of a combination of a sense strand nucleic acid and an antisense strand nucleic acid of the nucleotide sequence encoding an over-expressed protein encoded by a CDCA8 or AURKB gene. The term “siRNA” refers to a double stranded RNA molecule which prevents translation of a target mRNA. Standard techniques of introducing siRNA into the cell can be used in the treatment or prevention of the present invention, including those in which DNA is a template from which RNA is transcribed. The siRNA may be constructed such that a single transcript has both the sense and complementary antisense sequences from the target gene, e.g., a hairpin, which, in some embodiments, leads to production of micro RNA (miRNA). The siRNA may either be a dsRNA or shRNA.

As used herein, the term “dsRNA” refers to a construct of two RNA molecules comprising complementary sequences to one another and that have annealed together via the complementary sequences to form a double-stranded RNA molecule. The nucleotide sequence of two strands may comprise not only the “sense” or “antisense” RNAs selected from a protein coding sequence of target gene sequence, but also RNA molecule having a nucleotide sequence selected from non-coding rigion of the target gene.

As used herein, the term “complementary” refers to Watson Crick or Hoogsteen base pairing between nucleotides units of a nucleic acid molecule, and the term “binding” means the physical or chemical interaction between two nucleic acids or compounds or associated nucleic acids or compounds or combinations thereof. When the polynucleotide comprises modified nucleotides and/or non-phosphodiester linkages, these polynucleotides may also bind each other as same manner. Generally, complementary nucleic acid sequences hybridize under appropriate conditions to form stable duplexes containing few or no mismatches. For the purposes of this invention, two sequences having 5 or fewer mismatches are considered to be complementary. Furthermore, the sense strand and antisense strand of the isolated nucleotide of the present invention, can form double stranded nucleotide or hairpin loop structure by the hybridization.

The term “shRNA”, as used herein, refers to an siRNA having a stem-loop structure, comprising a first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a sense nucleic acid sequence (also referred to as “sense strand”), an antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules comprising complementary sequences to one another and that have annealed together via the complementary sequences to form a double-stranded polynucleotide molecule. The nucleotide sequence of two strands may comprise not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, comprising a first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shD/R-NA is a single-stranded region intervening between the sense and antisense strands and may also be referred to as “intervening single-strand”.

The method is used to suppress gene expression of a cell having up-regulated expression of a CDCA8 or AURKB gene. Binding of the siRNA to a CDCA8 or AURKB gene transcript in the target cell results in a reduction of a CDCA8 or AURKB protein production by the cell. The length of the oligonucleotide is at least about 10 nucleotides and may be as long as the naturally occurring transcript. Preferably, the oligonucleotide is about 75, about 50 or about 25 nucleotides in length. Most preferably, the oligonucleotide is less than about 19 to about 25 nucleotides in length.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxyabasic residue incorporation (US20060122137).

In another embodiment, modifications can be used to enhance the stability or to increase targeting efficiency of the double-stranded molecule. Modifications include chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deza, 7-alkyl, or 7-alkenyl purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the invention may comprise both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule consisting of a DNA strand (polynucleotide) and an RNA strand (polynucleotide), a chimera type double-stranded molecule comprising both DNA and RNA on any or both of the single strands (polynucleotides), or the like may be formed for enhancing stability of the double-stranded molecule. The hybrid of a DNA strand and an RNA strand may be the hybrid in which either the sense strand is DNA and the antisense strand is RNA, or the opposite so long as it has an activity to inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene.

In order to enhance stability of the double-stranded molecule, the molecule preferably contains as much DNA as possible, whereas to induce inhibition of the target gene expression, the molecule is required to be RNA within a range to induce sufficient inhibition of the expression. As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand.

That is, in preferable embodiments, a region flanking to the 3′-end of the antisense strand, or both of a region flanking to the 5′-end of sense strand and a region flanking to the 3′-end of antisense strand consists of RNA. For instance, the chimera or hybrid type double-stranded molecule of the present invention comprise following combinations.

| | | |
|---|---|---|
| sense strand: | 5′-[DNA]-3′ | |
| | 3′-(RNA)-[DNA]-5′: | antisense strand, |
| sense strand: | 5′-(RNA)-[DNA]-3′ | |
| | 3′-(RNA)-[DNA]-5′: | antisense strand, |
| and | | |
| sense strand: | 5′-(RNA)-[DNA]-3′ | |
| | 3′-(RNA)-5′: | antisense strand. |

The upstream partial region preferably is a domain consisting of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used, to silence gene expression via RNA interference. The shRNA or shD/R-NA comprises the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

Alternatively, a preferable siRNA used in the present invention has the general formula:

5′-[A]-[B]-[A′]-3′,

wherein [A] is a ribonucleotide sequence corresponding to a target sequence of a CDCA8 or AURKB gene; [B] is an intervening single strand, for example a ribonucleotide sequence consisting of about 3 to about 23 nucleotides; and [A′] is a ribonucleotide sequence complementary to [A]. Herein, the phrase a “target sequence of a CDCA8 or AURKB gene” refers to a sequence that, when introduced into NSCLC cell lines, is effective for suppressing cell viability.

The siRNA may optionally contain a 3′ overhang. A preferred siRNA is an siRNA that reduces the expression of a AURKB gene, wherein the siRNA has the nucleotide sequence of SEQ ID NO: 33, 59 or 60, in the sense strand as a target sequence. The siRNA has the general formula:

5′-[A]-[B]-[A′]-3′,

wherein [A] is a ribonucleotide sequence corresponding to SEQ ID NO: 33, 59 or 60; [B] is an intervening single strand, for example a ribonucleotide sequence composed of 3 to 23 nucleotides; and [A′] is a ribonucleotide sequence complementary to [A].

CCC, CCACC or CCACACC: Jacque, J. M, et al., (2002) Nature, Vol. 418: 435-8.

UUCG: Lee, N. S., et al., (2002) Nature Biotechnology 20:500-5. Fruscoloni, P., et al., (2003) Proc. Natl. Acad. Sci. USA 100(4): 1639-44.

UUCAAGAGA: Dykxhoorn, D. M., et al., (2002) Nature Reviews Molecular Cell Biology 4: 457-67.

Accordingly, the loop sequence can be selected from group consisting of, CCC, UUCG, CCACC, CCACACC, and UUCAAGAGA. Preferable loop sequence is UUCAAGAGA (“ttcaagaga” in DNA). Exemplary hairpin siRNA suitable for use in the context of the present invention include:

(for target sequence of SEQ ID NO: 33, 59 or 60)

for AURKB-siRNA

(SEQ ID NO: 48)

5′-GGTGATTCACAGAGACATA-[B]-TATGTCTCTGTGAATCACC-3′,

for target sequence is SEQ ID NO: 59
5′-CCAAACTGCTCAGGCATAA-[B]-TTATGCCTGAGCAGTTTGG-3′
and
for target sequence is SEQ ID NO: 60.

5′-ACGCGGCACTTCACAATTG-[B]-CAATTGTGAAGTGCCGCGT-3′.

Furthermore, the nucleotide sequence of siRNAs may be designed using a siRNA design computer program available from the Ambion website (http://www.ambion.com/techlib/misc/siRNA_finder.html). The nucleotide sequences for the siRNA may be selected by a computer program based on the following protocol:

Selection of siRNA Target Sites:
1. Beginning with the AUG start codon of the transcript, scan downstream for AA dinucleotide sequences. Record the occurrence of each AA and the 3′ adjacent 19 nucleotides as potential siRNA target sites. Tuschl, et al. Genes Dev 13(24): 3191-7 (1999), not recommend against designing siRNA against the 5′ and 3′ untranslated regions (UTRs) and regions near the start codon (within 75 bases) as these may be richer in regulatory protein binding sites, and thus the complex of endonuclease and siRNAs that were designed against these regions may interfere with the binding of UTR-binding proteins and/or translation initiation complexes.
2. Compare the potential target sites to the human genome database and eliminate from consideration any target sequences with significant homology to other coding sequences. The homology search can be performed using BLAST (Altschul S F, et al., Nucleic Acids Res. 1997; 25: 3389-402; J Mol. Biol. 1990; 215:403-10), which can be found on the NCBI server at: www.ncbi.nlm.nih.gov/BLAST/
3. Select qualifying target sequences for synthesis. On the website of Ambion, several preferable target sequences can be selected along the length of the gene for evaluation.

Transfection of vectors expressing siRNA polynucleotides of the invention can be used to inhibit growth of NSCLC cells. Thus, it is another aspect of the present invention to provide a double-stranded molecule composed of a sense-strand and antisense-strand which molecule functions as an siRNA for CDCA8 or AURKB, and a vector encoding the double-stranded molecule.

The double-stranded molecule of the present invention includes a sense strand and an antisense strand, wherein the sense strand is a ribonucleotide sequence corresponding to a CDCA8 or AURKB target sequence, and wherein the antisense strand is a ribonucleotide sequence which is complementary to the sense strand, wherein the sense strand and the antisense strand hybridize to each other to form the double-stranded molecule, and wherein the double-stranded molecule, when introduced into a cell expressing a CDCA8 or AURKB gene, inhibits expression of the gene.

The double-stranded molecule of the present invention may be a polynucleotide derived from its original environment (i.e., when it is a naturally occurring molecule, the natural environment), physically or chemically altered from its natural state, or chemically synthesized. According to the present invention, such double-stranded molecules include those composed of DNA, RNA, and derivatives thereof. A DNA is suitably composed of bases such as A, T, C and G, and T is replaced by U in an RNA.

For example, cells expressing the AURKB gene (e.g., LC319 cell) may be incubated with the oligonucleotide. Reagents such as Lipofectamine that help the introduction of the oligonucleotides into the cells may be added to the incubation mixture. Then, the inhibitory effect of the oligonucleotides on cell growth may be determined by comparison to the cell growth of cells incubated without the oligonucleotides. Alternatively, the inhibitory effect of oligonucleotides may be examined by administering the oligonucleotides into experimental animals, such as rats and mice with malignant neoplasms, to confirm decreased AURKB gene expression or decreased tumor cell growth in vivo.

The vector of the present invention preferably includes a regulatory sequence adjacent to the region encoding the present double-stranded molecule that directs the expression of the molecule in an adequate cell. For example, the double-stranded molecules of the present invention are intracellularly transcribed by cloning their coding sequence into a vector containing, e.g., a RNA polymerase III transcription unit from the small nuclear RNA (snRNA) U6 or the human H1 RNA promoter.

Alternatively, the present vectors may be produced, for example, by cloning the target sequence into an expression vector so the objective sequence is operatively-linked to a regulatory sequence of the vector in a manner to allow expression thereof (transcription of the DNA molecule) (Lee, N. S. et al., Nature Biotechnology 20: 500-5 (2002)). For example, the transcription of an RNA molecule having an antisense sequence to the target sequence may be driven by a first promoter (e.g., a promoter sequence linked to the 3′-end of the cloned DNA) and that having the sense strand to the target sequence by a second promoter (e.g., a promoter sequence linked to the 5′-end of the cloned DNA). The expressed sense and antisense strands hybridize to each other in vivo to generate a siRNA construct to silence a gene that contains the target sequence. Furthermore, two constructs (vectors) may be utilized to respectively produce the sense and anti-sense strands of a siRNA construct.

For introducing the vectors into a cell, transfection-enhancing agent can be used. FuGENE6 (Roche diagnostics), Lipofectamine 2000 (Invitrogen), Oligofectamine (Invitrogen), and Nucleofector (Wako pure Chemical) are useful as the transfection-enhancing agent.

The nucleic acids that inhibit CDCA8 or AURKB also include ribozymes against such gene(s). In the context of the present invention, ribozymes inhibit the expression of the over-expressed CDCA8 or AURKB protein and are thereby useful for suppressing the biological activity of such protein. Therefore, a composition composed of such a ribozyme is useful in treating or preventing NSCLC.

Generally, ribozymes are classified into large ribozymes and small ribozymes. A large ribozyme is known as an enzyme that cleaves the phosphate ester bond of nucleic acids. After the reaction with the large ribozyme, the reacted site consists of a 5′-phosphate and 3′-hydroxyl group. The large ribozyme is further classified into (1) group I intron RNA catalyzing transesterification at the 5′-splice site by guanosine; (2) group II intron RNA catalyzing self-splicing through a two step reaction via lariat structure; and (3) RNA component of the ribonuclease P that cleaves the tRNA precursor at the 5′ site through hydrolysis. On the other hand, small ribozymes have a smaller size (about 40 bp) as compared to the large ribozymes and cleave RNAs to generate a 5′-hydroxyl group and a 2′-3′ cyclic phosphate. Hammerhead type ribozymes (Koizumi et al., FEBS Lett. 228: 225 (1988)) and hairpin type ribozymes (Buzayan, Nature 323: 349-53 (1986); Kikuchi and Sasaki, Nucleic Acids Res. 19: 6751-5 (1991)) are included in the small ribozymes. Methods for designing and constructing ribozymes are known in the art (see Koizumi et al., FEBS Lett. 228: 228 (1988); Koizumi et al., Nucleic Acids Res. 17: 7059-71 (1989); Kikuchi and Sasaki, Nucleic Acids Res. 19: 6751-5 (1991)) and ribozymes inhibiting the expression of an over-expressed NSC protein can be constructed based on the sequence information of the nucleotide sequence encoding a CDCA8 or AURKB protein according to conventional methods for producing ribozymes.

Alternatively, the function of CDCA8 or AURKB can be inhibited by administering a compound that binds to or otherwise inhibits the function of the gene products. An example of such a compound is an antibody that binds to the over-expressed gene product or gene products.

The present invention refers to the use of antibodies, particularly antibodies against a protein encoded by any of the up-regulated genes CDCA8 or AURKB, or a fragment of such an antibody. As noted above, the term “antibody” refers to an immunoglobulin molecule having a specific structure that interacts (binds) specifically with an antigen used for synthesizing the antibody (i.e., the up-regulated gene product) or with an antigen closely related to it. An antibody that binds to the over-expressed CDCA8 or AURKB nucleotide may be in any form, such as monoclonal or polyclonal antibodies, and includes antiserum obtained by immunizing an animal such as a rabbit with the polypeptide, all classes of polyclonal and monoclonal antibodies, human antibodies and humanized antibodies produced by genetic recombination. Furthermore, the antibody used in the method of treating or preventing NSCLC of the present invention may be a fragment of an antibody or a modified antibody, so long as it binds to one or more of the proteins encoded by the marker genes (a CDCA8 or AURKB gene). The antibodies and antibody fragments used in the context of the present method of treating or preventing NSCLC may be modified, and include chemically modified and chimeric antibodies. Such antibodies and antibody fragments can be obtained according to the above-mentioned methods, supra.

When the obtained antibody is to be administered to the human body (antibody treatment), a human antibody or a humanized antibody is preferable for reducing immunogenicity. For example, transgenic animals having a repertory of human antibody genes may be immunized with an antigen such as a CDCA8 or AURKB polypeptide, cells expressing the polypeptide, or their lysates. Antibody producing cells are then collected from the animals and fused with myeloma cells to obtain hybridoma, from which human antibodies against the polypeptide can be prepared (see WO92-03918, WO94-02602, WO94-25585, WO96-33735, and WO96-34096).

Alternatively, an immune cell, such as an immunized lymphocyte, producing antibodies may be immortalized by an oncogene and used for preparing monoclonal antibodies. The present invention provides a method for treating or preventing NSCLC, using an antibody against an over-expressed a CDCA8 or AURKB polypeptide. According to the method, a pharmaceutically effective amount of an antibody against a CDCA8 or AURKB polypeptide is administered. An antibody against an over-expressed CDCA8 or AURKB polypeptide is administered at a dosage sufficient to reduce the activity of a CDCA8 or AURKB protein. Alternatively, an antibody binding to a cell surface marker specific for tumor cells can be used as a tool for drug delivery. Thus, for example, an antibody against an over-expressed CDCA8 or AURKB polypeptide conjugated with a cytotoxic agent may be administered at a dosage sufficient to injure tumor cells.

In addition, dominant negative mutants of the proteins disclosed here can be used to treat or prevent NSCLC. For example, the present invention provides methods for treating or preventing NSCLC in a subject by administering a CDCA8 mutant having a dominant negative effect, or a polynucleotide encoding such a mutant. The CDCA8 mutant may include an amino acid sequence that includes an AURKB binding region, e.g. a part of CDCA8 protein and included two phosphorylation sites, Ser-154, Ser-219, Ser-275, and Thr-278, by AURKB. The CDCA8 mutant may have the amino acid sequence of SEQ ID NO: 5.

In some preferred embodiments, the CDCA8 mutant is linked to a membrane transducing agent. A number of peptide sequences have been characterized for their ability to translocate into live cells and can be used for this purpose in the present invention. Such membrane transducing agents (typically peptides) are defined by their ability to reach the cytoplasmic and/or nuclear compartments in live cells after internalization. Examples of proteins from which transducing agents may be derived include HIV Tat transactivator1, 2, the Drosophila melanogaster transcription factor Antennapedia3. In addition, nonnatural peptides with transducing activity have been used. These peptides are typically small peptides known for their membrane-interacting properties which are tested for translocation. The hydrophobic region within the secretion signal sequence of K-fibroblast growth factor (FGF), the venom toxin mastoparan (transportan) 13, and Buforin I14 (an amphibian antimicrobial peptide) have been shown to be useful as transducing agents. For a review of transducing agents useful in the present invention see Joliot et al. Nature Cell Biology 6:189-96 (2004).

The CDCA8 mutant may have the general formula:

[R]-[D],

wherein [R] is a membrane transducing agent, and [D] is a polypeptide having the amino acid sequence of SEQ ID NO: 5. In the general formula, [R] may directly link with [D], or indirectly link with [D] through a linker. Peptides or compounds having plural functional groups may be used as the linker. Specifically, an amino acid sequence of -GGG- may be used as the linker. Alternatively, the membrane transducing agent and the polypeptide having the amino acid sequence of SEQ ID NO: 5 can bind to the surface of micro-particle. In the present invention, [R] may link with arbitral region of [D]. For example, [R] may link with N-terminus or C-terminus of [D], or side chain of the amino acid residues constituting [D]. Furthermore, plural molecules of [R] may also link with one molecule of [D]. In some embodiments, plural molecules of [R]s may link with different site of [D]. In another embodiments, [D] may be modified with some [R]s linked together.

The membrane transducing agent can be selected from group listed below;
[poly-arginine]; Matsushita, M. et al, J Neurosci. 21, 6000-7 (2003).
[Tat/RKKRRQRRR] (SEQ ID NO: 6) Frankel, A. et al, Cell 55, 1189-93 (1988).
Green, M. & Loewenstein, P. M. Cell 55, 1179-88 (1988).
[Penetratin/RQIKIWFQNRRMKWKK] (SEQ ID NO: 7)
Derossi, D. et al, J. Biol. Chem. 269, 10444-50 (1994).
[Buforin II/TRSSRAGLQFPVGRVHRLLRK] (SEQ ID NO: 8)
Park, C. B. et al. Proc. Natl Acad. Sci. USA 97, 8245-50 (2000).
[Transportan/GWTLNSAGYLLGKINLKALAALAKKIL] (SEQ ID NO: 9)
Pooga, M. et al. FASEB J. 12, 67-77 (1998).
[MAP (model amphipathic peptide)/KLALKLALKALKAALKLA] (SEQ ID NO: 10)
Oehlke, J. et al. Biochim. Biophys. Acta. 1414; 127-39 (1998).
[K-FGF/AAVALLPAVLLALLAP] (SEQ ID NO: 11)
Lin, Y. Z. et al. J. Biol. Chem. 270, 14255-14258 (1995).
[Ku70/VPMLK] (SEQ ID NO: 12)
Sawada, M. et al. Nature Cell Biol. 5, 352-7 (2003).
[Ku70/PMLKE] (SEQ ID NO: 13)
Sawada, M. et al. Nature Cell Biol. 5, 352-7 (2003).
[Prion/MANLGYWLLALFVTMWTDVGLCKKRPKP] (SEQ ID NO: 14)
Lundberg, P. et al. Biochem. Biophys. Res. Commun. 299, 85-90 (2002).
[pVEC/LLIILRRRIRKQAHAHSK] (SEQ ID NO: 15)
Elmquist, A. et al. Exp. Cell Res. 269, 237-44 (2001).
[Pep-1/KETWWETWWTEWSQPKKKRKV] (SEQ ID NO: 16)
Morris, M. C. et al. Nature Biotechnol. 19, 1173-6 (2001).
[SynB1/RGGRLSYSRRRFSTSTGR] (SEQ ID NO: 17)
Rousselle, C. et al. Mol. Pharmacol. 57, 679-86 (2000).
[Pep-7/SDLWEMMMVSLACQY] (SEQ ID NO: 18)
Gao, C. et al. Bioorg. Med. Chem. 10, 4057-65 (2002).
[HN-1/TSPLNIHNGQKL] (SEQ ID NO: 19)
Hong, F. D. & Clayman, G. L. Cancer Res. 60, 6551-6 (2000).

In the present invention, number of arginine residues that constitute the poly-arginine is not limited. In some preferred embodiments, 5 to 20 contiguous arginine residues may be exemplified. In a preferred embodiment, the number of arginine residues of the poly-arginine is 11 (SEQ ID NO: 20).

As used herein, the phrase “dominant negative fragment of CDCA8” refers to a mutated form of CDCA8 that is capable of complexing with AURKB. Thus, a dominant negative fragment is one that is not functionally equivalent to the full length CDCA8 polypeptide. Preferred dominant negative fragments are those that include an AURKB binding region, e.g. a part of CDCA8 protein and included two phosphorylation sites, Ser-154, Ser-219, Ser-275, and Thr-278, by AURKB.

### Pharmaceutical Compositions for Treating or Preventing NSCLC:

The present invention also provides compositions for treating or preventing NSCLC that include a compound selected by the present method of screening for a compound that alters the expression or activity of a CDCA8 or AURKB gene. For instance, the present invention provides a composition for treating or preventing NSCLC, said composition containing a pharmaceutically effective amount of an inhibitor having at least any one function selected from the group consisting of:

i. inhibiting a binding between CDCA8 and AURKB;

ii. inhibiting a phosphorylation of CDCA8 by AURKB;

iii. inhibiting a transcription of either of CDCA8 and AURKB genes, or both; and

iv. inhibiting CDCA8 stabilization by AURKB.

Alternatively, the present invention provides use of an inhibitor having at least any one function selected from the group consisting of:

i. inhibiting a binding between CDCA8 and AURKB;

ii. inhibiting a phosphorylation of CDCA8 by AURKB;

iii. inhibiting a transcription of either of CDCA8 and AURKB genes, or both; and

iv. inhibiting CDCA8 stabilization by AURKB,

for manufacturing pharmaceutical composition for treating or preventing NSCLC.

The present invention further provides of an inhibitor having at least any one function selected from the group consisting of:

i. inhibiting a binding between CDCA8 and AURKB;

ii. inhibiting a phosphorylation of CDCA8 by AURKB; and

iii. inhibiting a transcription of either of CDCA8 and AURKB genes, or both; and

iv. inhibiting CDCA8 stabilization by AURKB,

for treating or preventing NSCLC.

In a preferred embodiment, a compound or agent that specifically inhibits CDCA8 or AURKB may be used as inhibitors in the present invention. The term “specifically inhibit” in the context of inhibitory polynucleotides and polypeptides refers to the ability of an agent or ligand to inhibit the expression or the biological function of CDCA8 and/or AURKB. Specific inhibition typically results in at least about a 2-fold inhibition over background, preferably greater than about 10 fold and most preferably greater than 100-fold inhibition of CDCA8 and/or AURKB expression (e.g., transcription or translation) or measured biological function (e.g., cell growth or proliferation, inhibition of apoptosis, intracellular signaling from CDCA8, for example, phosphorylation by AURKB. Expression levels and/or biological function can be measured in the context of comparing treated and untreated cells, or a cell population before and after treatment. In some embodiments, the expression or biological function of CDCA8 and/or AURKB is completely inhibited. Typically, specific inhibition is a statistically meaningful reduction in CDCA8 and/or AURKB expression or biological function (e.g., p≦0.05) using an appropriate statistical test.

Such active ingredient inhibiting a transcription of either of CDCA8 and AURKB genes (iii) and (iv) can also be an antisense-oligonucleotide, siRNA or ribozyme against the gene, or derivatives, such as expression vector, of the antisense-oligonucleotide, siRNA or ribozyme, as described above. Alternatively, active ingredient inhibiting a phosphorylation of CDCA8 by AURKB (ii) and (iv) can be a dominant negative mutants of CDCA8 as described above. Further, an antagonists of CDCA8 can be used as active ingredient inhibiting a binding between CDCA8 and AURKB (i). Alternatively, such active ingredient may be selected by the screening method as described above.

When administering a compound isolated by the screening method of the present invention as a pharmaceutical for humans and other mammals, such as mice, rats, guinea-pig, rabbits, cats, dogs, sheep, pigs, cattle, monkeys, baboons or chimpanzees for treating a cell proliferative disease (e.g., non-small cell lung cancer), the isolated compound can be directly administered or can be formulated into a dosage form using conventional pharmaceutical preparation methods. Such pharmaceutical formulations of the present compositions include those suitable for oral, rectal, nasal, topical (including buccal and sub-lingual), vaginal or parenteral (including intramuscular, sub-cutaneous and intravenous) administration, or for administration by inhalation or insufflation.

For example, according to the need, the agents can be taken orally, as sugar-coated tablets, capsules, elixirs and microcapsules; or non-orally, in the form of injections of sterile solutions or suspensions with water or any other pharmaceutically acceptable liquid. For example, the agents can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredients in these preparations makes a suitable dosage within the indicated range acquirable.

The preparations may be optionally packaged in discrete dosage units. Pharmaceutical formulations suitable for oral administration include, but are not limited to, capsules, cachets or tablets, each containing a predetermined amount of the active ingredient. Illustrative formulations further include powders, granules, solutions, suspensions and emulsions. The active ingredient is optionally administered as a bolus electuary or paste. Tablets and capsules suitable for oral administration may contain conventional excipients, such as binding agents, fillers, lubricants, disintegrants and/or wetting agents. A tablet may be made by compression or molding, optionally with one or more formulational ingredients. Compressed tablets may be prepared by compressing in a suitable machine the active ingredients in a free-flowing form such as powder or granules, optionally mixed with a binder, lubricant, inert diluent, lubricating, surface active or dispersing agent. Molded tablets may be made via molding in a suitable machine a mixture of the powdered compound moistened with an inert liquid diluent. The tablets may be coated according to methods well known in the art.

Oral fluid preparations may be in the form of, for example, aqueous or oily suspensions, solutions, emulsions, syrups or elixirs, or may be presented as a dry product for reconstitution with water or other suitable vehicle prior to use. Such liquid preparations may contain conventional additives such as suspending agents, emulsifying agents, non-aqueous vehicles (which may include edible oils) or preservatives. The tablets may optionally be formulated so as to provide slow or controlled release of the active ingredient in vivo. A package of tablets may contain one tablet to be taken on each of the month. The formulation or dose of medicament in these preparations makes a suitable dosage within the indicated range acquirable.

Exemplary formulations for parenteral administration include aqueous and non-aqueous sterile injection solutions which optionally contain anti-oxidants, buffers, bacteriostats and solutes which render the formulation isotonic with the blood of the intended recipient; and aqueous and non-aqueous sterile suspensions which may include, but are not limited to, suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Exemplary formulations for rectal administration include suppositories with standard carriers such as cocoa butter or polyethylene glycol. Formulations for topical administration in the mouth, for example, buccally or sublingually, include lozenges, which contain the active ingredient in a flavored base such as sucrose and acacia or tragacanth, and pastilles including the active ingredient in a base such as gelatin, glycerin, sucrose or acacia. For intra-nasal administration of an active ingredient, a liquid spray or dispersible powder or in the form of drops may be used. Drops may be formulated with an aqueous or non-aqueous base also including one or more dispersing agents, solubilizing agents or suspending agents.

For administration by inhalation, the compositions may be conveniently delivered from an insufflator, nebulizer, pressurized packs or other convenient means of delivering an aerosol spray. Pressurized packs may include a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compositions may take the form of a dry powder composition, for example, a powder mix of an active ingredient and a suitable powder base such as lactose or starch. The powder composition may be presented in unit dosage form in, for example, capsules, cartridges, gelatin or blister packs from which the powder may be administered with the aid of an inhalator or insufflators.

Other suitable formulations include implantable devices and adhesive patches; which release a therapeutic agent.

When desired, the above-described formulations may be adapted to provide sustained release of the active ingredient. The pharmaceutical compositions may also contain other active ingredients, including, but not limited to, antimicrobial agents, immunosuppressants and preservatives.

It should be understood that in addition to the ingredients particularly mentioned above, the formulations of this invention may include other agents conventional in the art having regard to the type of formulation in question; for example, those suitable for oral administration may include flavoring agents.

Preferred unit dosage formulations are those containing an effective dose, as recited below, of the active ingredient or an appropriate fraction thereof.

Methods well known to one skilled in the art may be used to administer an agent identified by the screening methods of the present methods to patients, for example, as intraarterial, intravenous, or percutaneous injections and also as intranasal, intramuscular or oral administrations. The dose employed will depend upon a number of factors, including the age and sex of the subject, the precise disorder being treated, and its severity. Also the route of administration may vary depending upon the condition and its severity. For example, if said agent is encodable by a DNA, the DNA can be inserted into a vector for gene therapy and the vector administered to a patient to perform the therapy.

For each of the aforementioned conditions, the compositions, e.g., polypeptides and organic compounds, may be administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

As noted above, the present invention further provides a composition for treating or preventing NSCLC that contains an active ingredient that inhibits the expression of the over-expressed genes. The active ingredient may be made into an external preparation, such as liniment or a poultice, by mixing with a suitable base material which is inactive against the derivatives.

Also, as needed, the active ingredient can be formulated into tablets, powders, granules, capsules, liposome capsules, injections, solutions, nose-drops and freeze-drying agents by adding excipients, isotonic agents, solubilizers, preservatives, pain-killers and such. These can be prepared according to conventional methods for preparing nucleic acid containing pharmaceuticals.

Preferably, the antisense-oligonucleotide derivative, siRNA derivative or ribozyme derivative is given to the patient by direct application to the ailing site or by injection into a blood vessel so that it will reach the site of ailment. A mounting medium can also be used in the composition to increase durability and membrane-permeability. Examples of mounting mediums include liposome, poly-L-lysine, lipid, cholesterol, lipofectin and derivatives thereof.

The dosage of such compositions can be adjusted suitably according to the patient's condition and used in desired amounts. For example, a dose range of 0.1 to 100 mg/kg, preferably 0.1 to 50 mg/kg can be administered.

Another embodiment of the present invention is a composition for treating or preventing NSCLC composed of an antibody against a CDCA8 or AURKB polypeptide or fragments of the antibody that bind to the polypeptide.

Although dosages may vary according to the symptoms, an exemplary dose of an antibody or fragments thereof for treating or preventing NSCLC is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the condition of the patient, symptoms of the disease and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kg of body-weight.

### Method for Assessing an NSCLC Prognosis:

The differentially expressed CDCA8 or AURKB gene identified herein can also allow for prognosis, testing or monitoring the course of treatment of NSCLC. In this method, a test biological sample is provided from a subject undergoing treatment for NSCLC. If desired, multiple test biological samples are obtained from the subject at various time points, for example, before, during or after the treatment. The expression level of one or more of a CDCA8 or AURKB gene in the sample is then determined and compared to a reference sample with a known state of NSCLC that has not been exposed to the treatment. In some preferred embodiments of the present invention, the expression level of both of CDCA8 and AURKB gene may be detected.

In the context of the present invention, determination of a poor prognosis may be used to determine further treatment, e.g., to stop further treatments that reduce quality of life, to treat the cancer in a different manner than previously used, or to treat the cancer more aggressively. In other words, the assessment of a prognosis enables clinicians to choose, in advance, the most appropriate treatment for an individual NSCLC patient without even the information of conventional clinical staging of the disease, using only routine procedures for tissue-sampling.

Further, the methods of the present invention may be used to assess the efficacy of a course of treatment. For example, in a mammal with cancer from which a biological sample is found to contain an elevated level of CDCA8 and/or AURKB expression, the efficacy of an anti-cancer treatment can be assessed by monitoring the CDCA8 and AURKB expression levels over time. For example, a decrease in CDCA8 and/or AURKB expression level in a biological sample taken from a mammal following a course of treatment, as compared to a level observed in a sample taken from the mammal before treatment onset, or earlier in, the treatment, may be indicative of efficacious treatment.

Alternatively, according to the present invention, an intermediate result may also be provided in addition to other test results for assessing the prognosis of a subject. Such intermediate result may assist a doctor, nurse, or other practitioner to assess, determine, or estimate the prognosis of a subject. Additional information that may be considered, in combination with the intermediate result obtained by the present invention, to assess prognosis includes clinical symptoms and physical conditions of a subject.

In the context of the present invention, if a reference sample contains no NSCLC cells, a similarity in the expression level of the CDCA8 or AURKB gene in the test biological sample and the reference sample indicates the efficaciousness of the treatment. However, a difference in the expression level of a CDCA8 or AURKB gene in the test as compared to the reference samples indicates a less favorable clinical outcome or prognosis.

In the context of the present invention, NSCLC cells obtained from patients with a favorable prognosis may be used as the reference sample. For example, generally, when a patient could survive more than five years after the surgery, the patient had favorable prognosis. More specifically, long survivors (i.e. favorable prognosis) and short survivors (i.e. poor prognosis) groups include patients whose average 5-years tumor-specific survival rate was more than 69% and less than 45%, respectively. Thus, samples derived from such short survivors, and showing strong staining can be used as a positive control for poor prognosis.

Alternatively, instead of the patient derived samples, samples or lung cancer cell lines showing strong staining similar to the patient derived samples can be also used as the positive control. Furthermore, in some embodiments, normal lung cells, lung cancer cells or other cells with no expression of CDCA8 and AURKB can be used as negative controls for poor prognosis.

The present invention also includes kits for assaying and assessing a NSCLC prognosis, wherein the kit includes one or more of the components selected from the group consisting of:
(a) a reagent for detecting the presence of an mRNA encoding the amino acid sequence of SEQ ID NO: 2 (CDCA8) or SEQ ID NO: 4 (AURKB),
(b) a reagent for detecting the presence of a protein having the amino acid sequence of SEQ ID NO: 2 (CDCA8) or SEQ ID NO: 4 (AURKB), and
(c) a reagent for detecting the biological activity of a protein having the amino acid sequence of SEQ ID NO: 2 (CDCA8) or SEQ ID NO: 4 (AURKB).

In some preferred embodiments, (a) a reagent for detecting the presence of an mRNA encoding the amino acid sequence of SEQ ID NO: 2 (CDCA8) or SEQ ID NO: 4 (AURKB) may be a nucleic acid that specifically binds to or identifies CDCA8 or AURKB nucleic acids, such as oligonucleotide sequences which are complementary to a CDCA8 or AURKB nucleic acid. Specifically, amino acid sequence of SEQ ID NO: 2 (CDCA8) and SEQ ID NO: 4 (AURKB) are encoded by nucleotide sequence of SEQ ID NO: 1 and SEQ ID NO: 3. Thus, an oligonucleotide that includes the nucleotide sequence selected from nucleotide sequence of SEQ ID NO: 1 and SEQ ID NO: 3 may be used as preferable primer or probe of the present invention. Alternatively, in the present invention, (b) a reagent for detecting the presence of a protein including the amino acid sequence of SEQ ID NO: 2 (CDCA8) or SEQ ID NO: 4 (AURKB) may be an antibody that bind to CDCA8 or AURKB proteins.

Furthermore, the biological activity of CDCA8 or AURKB, e.g. CDCA8's AURKB-dependent phosphorylation at Ser-154, Ser-219, Ser-275, and/or Thr-278, may also detected using any suitable assay method. The detection reagents may be packaged together in the form of a kit. The reagents are preferably packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay may also be included in the kit. The assay format of the kit may be a Northern hybridization or a sandwich ELISA, both of which are known in the art.

For example, the detection reagent may be immobilized on a solid matrix such as a porous strip to form at least one NSCLC detection site. The measurement or detection region of the porous strip may include a plurality of sites, each containing a nucleic acid. A test strip may also contain sites for negative and/or positive controls. Alternatively, control sites may be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the prognosis of the sample. The detection sites may be configured in any suitably detectable shape and are typically in the shape of a bar or dot spanning the width of a test strip.

In a preferred embodiment, the kit of the present invention, in addition to at lease one element selected from (a) to (c), may further comprises (d) either or both of positive control and negative control sample derived from patients having poor or favorable prognosis respectively. In particular, fixed lung cells or tissue collected from NSCLC foci of the patients may be used for these control samples for immunostaining.

Hereinafter, the present invention is described in more detail by reference to the Examples. However, the following materials, methods and examples are presented only to illustrate the present invention and to assist one of ordinary skill in making and using the same. The examples are not intended in any way to otherwise limit the scope of the invention. As such, methods and materials similar or equivalent to those described herein can be used in the practice or testing of the present invention.

### EXAMPLES

### Materials and Methods

### (a) Lung-Cancer Cell Lines and Tissue Samples:

The human lung-cancer cell lines used in the instant Examples were as follows: lung adenocarcinomas (ADC), A427, A549, LC319, PC14, and NCI-H1373; lung squamous-cell carcinomas (SCC), SK-MES-1, EBC-1, and NCI-H226; and small-cell lung cancers (SCLC), DMS114, DMS273, SBC-3, and SBC-5. Human lung derived cells used in the instant. Examples were as follows: MRC-5 (fibroblast), CCD-19Lu (fibroblast), and BEAS-2B (lung epithelia, bronchus), which were purchased from the American Type Culture Collection (ATCC; Manassas, Va.).

All cells were grown in monolayers in appropriate medium supplemented with 10% fetal calf serum (FCS) and were maintained at 37 degrees C. in atmospheres of humidified air with 5% CO₂. Human small airway epithelial cells (SAEC) were grown in optimized medium (SAGM) purchased from Cambrex Bio Science Inc. (Walkersville, Md.). Primary lung cancer samples were obtained with written informed consent, as described previously (Kato T, et al., Cancer Res. 2005 Jul. 1; 65(13):5638-46). A total of 273 NSCLC and adjacent normal lung-tissue samples for immunostaining on tissue microarray analysis were also obtained from patients. The experiments described herein and the use of all clinical materials were approved by individual institutional ethical committees.

### (b) Semiquantitative RT-PCR:

Total RNA was extracted from cultured cells and clinical tissues using Trizol reagent (Life Technologies, Inc., Gaithersburg, Md.) according to the manufacturer's protocol. Extracted RNAs and normal human tissue poly(A) RNAs were treated with DNase I (Nippon Gene, Tokyo, Japan) and reversely-transcribed using oligo (dT) primer and SuperScript II reverse transcriptase (Invitrogen, Carlsbad, Calif.). Semi-quantitative RT-PCR experiments were carried out with the following synthesized CDCA8-specific primers, AURKB-specific primers, E2F-1-specific primers, or with beta-actin (ACTB)-specific primers as an internal control:

| | |
|---|---|
| CDCA8, | |
| 5′-CATCTGGCATTTCTGCTCTCTAT-3′ | (SEQ ID NO: 21) |
| and | |
| 5′-CTCAGGGAAAGGAGAATAAAAGAAC-3′; | (SEQ ID NO: 22) |
| AURKB, | |
| 5′-CCCATCTGCACTTGTCCTCAT-3′ | (SEQ ID NO: 23) |
| and | |
| 5′-AACAGATAAGGGAACAGTTAGGGA-3′; | (SEQ ID NO: 24) |
| E2F-1, | |
| 5′-GGAGTCTGTGTGGTGTGTATGTG-3′ | (SEQ ID NO: 25) |
| and | |
| 5′-GAGGGAACAGAGCTGTTAGGAAG-3′; | (SEQ ID NO: 26) |
| ACTB, | |
| 5′-GAGGTGATAGCATTGCTTTCG-3′ | (SEQ ID NO: 27) |
| and | |
| 5′-CAAGTCAGTGTACAGGTAAGC-3′. | (SEQ ID NO: 28) |

PCR reactions were optimized for the number of cycles to ensure product intensity within the logarithmic phase of amplification.

### (c) Northern-Blot Analysis:

Human multiple-tissue blots containing 23 tissues (BD Biosciences Clontech, Palo Alto, Calif.) were hybridized with a ³²P-labeled PCR product of CDCA8. The cDNA probe of CDCA8 was prepared by RT-PCR using the primers described above. Pre-hybridization, hybridization, and washing were performed according to the supplier's recommendations. The blots were autoradiographed at room temperature for 30 hours with intensifying BAS screens (BIO-RAD, Hercules, Calif.).

### (d) Antibodies:

To obtain anti-CDCA8 antibody, plasmids were prepared expressing full-length of CDCA8 that contained His-tagged epitopes at their NH2 (N)-terminals using pET28 vector (Novagen, Madison, Wis.). The recombinant proteins were expressed in Escherichia coli, BL21 codon-plus strain (Stratagene, LaJolla, Calif.), and purified using TALON resin (BD Biosciences Clontech) according to the supplier's protocol. The protein, extracted on an SDS-PAGE gel, was inoculated into rabbits; the immune sera were purified on affinity columns according to standard methodology. Affinity-purified rabbit polyclonal anti-CDCA8 antibodies were used for western blotting and immunostaining. A rabbit polyclonal anti-AURKB antibody was purchased from abcam Inc. (Catalog No. ab2254; Cambridge, UK). On western blots, it was confirmed that the antibody was specific to CDCA8 or AURKB, using lysates from NSCLC cell lines that either expressed CDCA8 and AURKB endogenously or not, or cells transfected with CDCA8 or AURKB expression vector.

### (e) Western-Blotting:

Cells were lysed in lysis buffer; 50 mM Tris-HCl (pH 8.0), 150 mM NaCl, 0.5% NP-40, 0.5% deoxycholate-Na, 0.1% SDS, plus protease inhibitor (Protease Inhibitor Cocktail Set III; Calbiochem Darmstadt, Germany). An ECL western-blot analysis system was used (GE Healthcare Bio-sciences, Piscataway, N.J.), as previously described (Kato T, et al., Cancer Res. 2005 Jul. 1; 65(13):5638-46; Furukawa C, et al., Cancer Res. 2005 Aug. 15; 65(16):7102-10).

### (f) Immunocytochemistry:

Cultured cells were washed twice with PBS(−), fixed in 4% formaldehyde solution for 60 min at room temperature, and rendered permeable by treatment for 1.5 minutes with PBS(−) containing 0.1% Triton X-100. Cells were covered with 3% BSA in PBS(−) for 60 minutes to block non-specific binding prior to the primary antibody reaction. Then the cells were incubated with antibody to human CDCA8 or AURKB protein, or anti-FLAG monoclonal antibody (Sigma-Aldrich Co., St. Louis, Mo.). The immune complexes were stained with a goat anti-rabbit secondary antibody conjugated to Alexa594 (Molecular Probes, Eugene, Oreg.), and viewed with a laser confocal microscope (TCS SP2 AOBS: Leica Microsystems, Wetzlar, Germany).

To determine the cell cycle-dependent expression and localization of wild-type or mutant CDCA8 that was stably expressed in A549 cells, synchronization at the G1/S boundary was achieved by aphidicolin block, as previously described (Suzuki C, et al., Cancer Res. 2005 Dec. 15; 65(24):11314-25). Cells were treated with 1 microgram/ml of aphidicolin (Sigma-Aldrich, Co.) for 24 hours and released from the cell-cycle arrest by washes in PBS for four times. These cells were cultured in medium and harvested for analysis at 1.5 and 9 hours after the release of the cell-cycle arrest, and were used for immunoblotting and immunostaining.

### (g) Immunohistochemistry and Tissue-Microarray Analysis:

To investigate the CDCA8/AURKB protein in clinical materials, tissue sections were stained using ENVISION+Kit/HRP (DakoCytomation, Glostrup, Denmark). Affinity-purified anti-CDCA8 antibody or anti-AURKB antibody was added after blocking of endogenous peroxidase and proteins, and each section was incubated with HRP-labeled anti-rabbit IgG as the secondary antibody. Substrate-chromogen was added and the specimens were counterstained with hematoxylin.

Tumor-tissue microarrays were constructed as according to published protocols, using formalin-fixed NSCLCs (Chin S F, et al., Mol Pathol. 2003 October; 56(5):275-9; Callagy G, et al., Diagn Mol Pathol. 2003 March; 12(1):27-34; Callagy G, et al., J Pathol. 2005 February; 205(3):388-96). Positivity for CDCA8 and AURKB was assessed semi-quantitatively by three independent investigators without prior knowledge of the clinical follow-up data. The intensity of histochemical staining was recorded as absent (scored as 0), weak (1+), or strong (2+). When all scorers judged as strongly positive, the cases were scored as 2+.

### (h) Statistical Analysis:

Attempts were made to correlate clinicopathological variables, such as age, gender, and pathological TNM stage, with the expression levels of CDCA8 and AURKB protein determined by tissue-microarray analysis. Tumor-specific survival curves were calculated from the date of surgery to the time of death related to NSCLC, or to the last follow-up observation. Kaplan-Meier curves were calculated for each relevant variable and for CDCA8 or AURKB expression; differences in survival times among patient subgroups were analyzed using the log-rank test. Univariate analysis was performed with the Cox proportional-hazard regression model to determine associations between clinicopathological variables and cancer-related mortality.

### (i) RNA Interference Assay:

A vector-based RNA interference (RNAi) system, psiH1BX3.0, which was designed to synthesize siRNAs in mammalian cells was previously established (Suzuki C, et al., Cancer Res. 2003 Nov. 1; 63(21):7038-41; Kato T, et al., Cancer Res. 2005 Jul. 1; 65(13):5638-46; Furukawa C, et al., Cancer Res. 2005 Aug. 15; 65(16):7102-10; Suzuki C, et al., Cancer Res. 2005 Dec. 15; 65(24):11314-25. Ishikawa N, et al., Cancer Sci. 2006 August; 97(8):737-45; Takahashi K, et al., Cancer Res. 2006 Oct. 1; 66(19):9408-19; Hayama S, et al., Cancer Res. 2006 Nov. 1; 66(21):10339-48). 10 micrograms of siRNA-expression vector was transfected using 30 microliters of Lipofectamine 2000 (Invitrogen) into lung-cancer cell lines, LC319 and SBC-5. The transfected cells were cultured for seven days in the presence of appropriate concentrations of geneticin (G418), and the number of colonies was counted by Giemsa staining, and viability of cells was evaluated by MTT assay (cell-counting kit-8 solution; DOJINDO, Kumamoto, Japan), at 7 days after the G418 treatment. To confirm suppression of CDCA8 protein expression, western blotting was carried out with affinity-purified polyclonal antibody to CDCA8 according to the standard protocol. The target sequences of the synthetic oligonucleotides for RNAi were as follows:

control 1
(EGFP: enhanced green fluorescent protein (GFP)
gene, a mutant of Aequorea victoria GFP),

| | |
|---|---|
| 5′-GAAGCAGCACGACTTCTTC-3′; | (SEQ ID NO: 29) |

control 2
(Luciferase: Photinus pyralis luciferase gene),

| | |
|---|---|
| 5′-CGTACGCGGAATACTTCGA-3′; | (SEQ ID NO: 30) |
| si-CDCA8-#1, | |
| 5′-CAGCAGAAGCTATTCAGAC-3′; | (SEQ ID NO: 31) |
| si-CDCA8-#2, | |
| 5′-GCCGTGCTAACACTGTTAC-3′, | (SEQ ID NO: 32) |
| si-AURKB, | |
| 5′-GGTGATTCACAGAGACATA-3′. | (SEQ ID NO: 33) |

TABLE 1
Sequences of specific double-stranded oligonu-
cleotide inserted into siRNA expression vector
and target sequences of each siRNAs

| | | | SEQ ID |
|---|---|---|---|
| gene | | Nucleotide Sequence | NO: |
| EGEP | insert | TCCCGAAGCAGCACGACTTCTTCTTCAA | 34 |
| | | GAGAGAAGAAGTCGTGCTGCTTC | |
| EGFP | insert | AAAAGAAGCAGCACGACTTCTTCTCTCT | 35 |
| | | TGAAGAAGAAGTCGTGCTGCTTC | |
| EGFP | hairpin | GAAGCAGCACGACTTCTTCTTCAAGAG | 36 |
| | | AGAAGAAGTCGTGCTGCTTC | |
| LUC | insert | TCCCCGTACGCGGAATACTTCGATTCAA | 37 |
| | | GAGATCGAAGTATTCCGCGTACG | |
| LUC | insert | AAAACGTACGCGGAATACTTCGATCTCT | 38 |
| | | TGAATCGAAGTATTCCGCGTACG | |
| LUC | hairpin | CGTACGCGGAATACTTCGATTCAAGAG | 39 |
| | | ATCGAAGTATTCCGCGTACG | |
| CDCA8- | insert | TCCCCAGCAGAAGCTATTCAGACTTCAA | 40 |
| #1 | | GAGAGTCTGAATAGCTTCTGCTG | |
| CDCA8- | insert | AAAACAGCAGAAGCTATTCAGACTCTC | 41 |
| #1 | | TTGAAGTCTGAATAGCTTCTGCTG | |
| CDCA8- | hairpin | CAGCAGAAGCTATTCAGACTTCAAGAG | 42 |
| #1 | | AGTCTGAATAGCTTCTGCTG | |
| CDCA8- | insert | TCCCGCCGTGCTAACACTGTTACTTCAA | 43 |
| #2 | | GAGAGTAACAGTGTTAGCACGGC | |
| CDCA8- | insert | AAAAGCCGTGCTAACACTGTTACTCTCT | 44 |
| #2 | | TGAAGTAACAGTGTTAGCACGGC | |
| CDCA8- | hairpin | GCCGTGCTAACACTGTTACTTCAAGAGA | 45 |
| #2 | | GTAACAGTGTTAGCACGGC | |
| AURKB | insert | TCCCGGTGATTCACAGAGACATATTCAA | 46 |
| | | GAGATATGTCTCTGTGAATCACC | |
| AURKB | insert | AAAAGGTGATTCACAGAGACATATCTC | 47 |
| | | TTGAATATGTCTCTGTGAATCACC | |
| AURKB | hairpin | GGTGATTCACAGAGACATATTCAAGAG | 48 |
| | | ATATGTCTCTGTGAATCACC | |

Furthermore, to inhibit the AURKB activity in mammalian cells, siRNA oligos were constructed against AURKB (si-AURKB-#1 and -#2), as well as control siRNA oligos for EGFP. The target sequences of the synthetic oligonucleotides for RNAi were as follows:

| | |
|---|---|
| control 3 (EGFP), | |
| 5′-GAAGCAGCACGACTTCTTC-3′; | (SEQ ID NO: 58) |
| si-AURKB-#1, | |
| 5′-CCAAACTGCTCAGGCATAA-3′; | (SEQ ID NO: 59) |
| si-ARURKB-#2, | |
| 5′-ACGCGGCACTTCACAATTG-3′. | (SEQ ID NO: 60) |

Cells seeded onto 10 cm-dishes were incubated in mixtures of both Lipofectamine (Invitrogen) and control 3 (5′-GAAGCAGCACGACUUCUUC-3′ (SEQ ID NO: 61)) or si-AURKBs (#1: 5′-CCAAACUGCUCAGGCAUAA-3′ (SEQ ID NO: 62);

#2: 5′-ACGCGGCACUUCACAAUUG-3′ (SEQ ID NO: 63)) in a final concentration of 100 nM. At 4 hours after transfection, the siRNA-lipofectamine mixtures were replaced with fresh media. The cells were collected and analyzed after additional 72-hour culture.

### (j) Luciferase Assay:

Human genomic DNA was extracted from LC319 cells and used as templates for PCR. 5′ flanking region of the human CDCA8 or AURKB gene was amplified by PCR with the following synthesized:

5′ region of CDCA8-specific primers,

(SEQ ID NO: 49)

5′-CGGGGTACCCCGACAAGGCCTGCCGGGAGTAGT-3′
and

(SEQ ID NO: 50)

5′-CCCAAGCTTGGGCGAATCTGTGCAGCTCGTGTC-3′,
and
5′ region of AURKB-specific primers,

(SEQ ID NO: 51)

5′-AACGTAGGCATGTAGAGGCTC-3′
and

(SEQ ID NO: 52)

5′-CGGGGAAGAAAGTGCTTAAAGGA-3′.

The fragments of promoter region were excised with KpnI and HindIII restriction enzymes, and inserted into the corresponding enzyme sites of pGL3-Basic vector. The entire coding sequence of E2F-1 was cloned into the appropriate site of pcDNA3.1/myc-His plasmid vector (Invitrogen) to achieve pcDNA3.1-E2F-1. The plasmids containing the 5′ flanking region of the CDCA8 gene and phRL-SV40 (Promega, Madison, Wis.) were co-transfected into LC319 cells with pcDNA3.1-E2F-1 or mock vector using Lipofectamine Plus (Invitrogen). Firefly luciferase activity values were normalized by comparing firefly luciferase activity with Renilla luciferase activity, expressed from phRL-SV40 to allow variation in transfection efficiency.

### (k) Recombinant CDCA8 Proteins:

13 plasmids expressing wild-type CDCA8 or various deletion mutant CDCA8 proteins (CDCA8delta1-delta12), each of which was mutated at serine/threonine to alanine and contained His-tagged epitopes at their N-terminals were prepared using pET28 vector (Novagen). The recombinant proteins were expressed in Escherichia coli, BL21 codon-plus strain (Stratagene), and purified using TALON resin (BD Biosciences Clontech) according to the manufacturer's protocol.

### (l) In Vitro Kinase Assay:

Purified recombinant wild-type or mutated CDCA8 proteins were incubated with recombinant AURKB (Catalog No. 14-489; upstate, Lake Placid, N.Y.) and [gamma-³²P] ATP in kinase buffer (20 mM Tris, pH 7.5, 10 mM MgCl₂, 2 mM MnCl₂, 1 mM PMSF, and 1 mM dithiothreitol) supplemented with a mixture of protease inhibitors, 10 mM NaF, 5 nM microcystin LR, and 50 micromoles ATP. The reaction was terminated by the addition of a 0.2 volume of 5× protein sample buffer and the proteins were analyzed by SDS-PAGE.

### (m) Synthesized Dominant-Negative Peptide:

Dominant-negative 19 or 20 amino-acid peptide sequences corresponding to a part of CDCA8 protein that contained possible phosphorylation sites by AURKB was covalently linked at its N-terminus to a membrane transducing 11 poly-arginine sequence (11R) (Hayama S, et al., Cancer Res. 2006 Nov. 1; 66(21):10339-48; Matsushita M, et al., J Neurosci. 2001 Aug. 15; 21(16):6000-7). Three cell-permeable peptides were synthesized; 11R-CDCA8₁₄₇₋₁₆₅, RRRRRRRRRRR-GGG-PSKKRTQSIQGKGKGKRSS (SEQ ID NO: 53); 11R-CDCA8₂₀₉₋₂₂₈, RRRRRRRRRRR-GGG-ERIYNISGNGSPLADSKEIF (SEQ ID NO: 54); 11R-CDCA8₂₆₁₋₂₈₀, RRRRRRRRRRR-GGG-NIKKLSNRLAQICSSIRTHK (SEQ ID NO: 55). Peptides were purified by preparative reverse-phase HPLC.

LC319, SBC-5 cells and BEAS-2B cells were incubated with the 11R-peptides at the concentration of 2.5 micromoles, 5 micromoles, and 7.5 micromoles for seven days. The medium was exchanged at every 48 hours at the appropriate concentrations of each peptide and the viability of cells was evaluated by MTT assay at 7 days after the treatment. To confirm the transduction efficiency of the peptides, the 11R-CDCA8₂₆₁₋₂₈₀ and its control peptides labeled with fluorescein isothiocyanate (FITC) at N-terminus were synthesized. Transduction of the peptides (2.5-7.5 micromoles) was monitored by fluorescence microscopic observation after a 3-hour incubation of the peptides with the cell lines at 37 degrees C. The 11R-CDCA8₂₆₁₋₂₈₀ as well as its control peptides was transduced into almost all of cultured cells as reported elsewhere (Futaki S, et al. J Biol Chem 2001 276:5836-40).

### Results:

### (a) Co-Activation of the CDCA8 and AURKB in Lung Cancers:

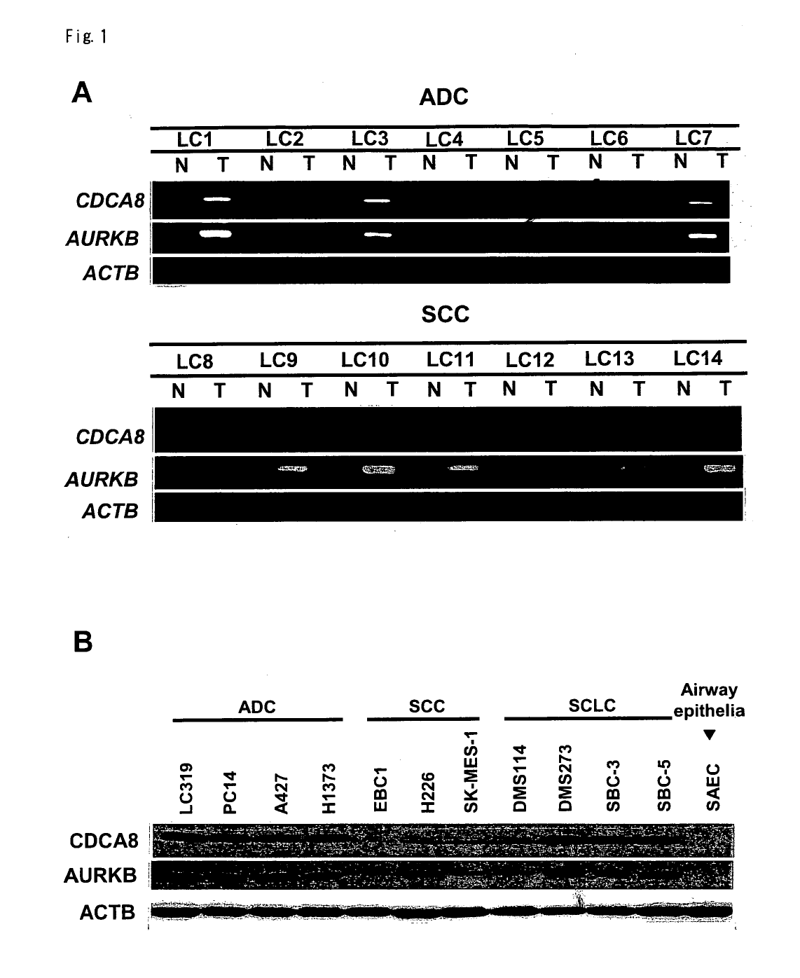

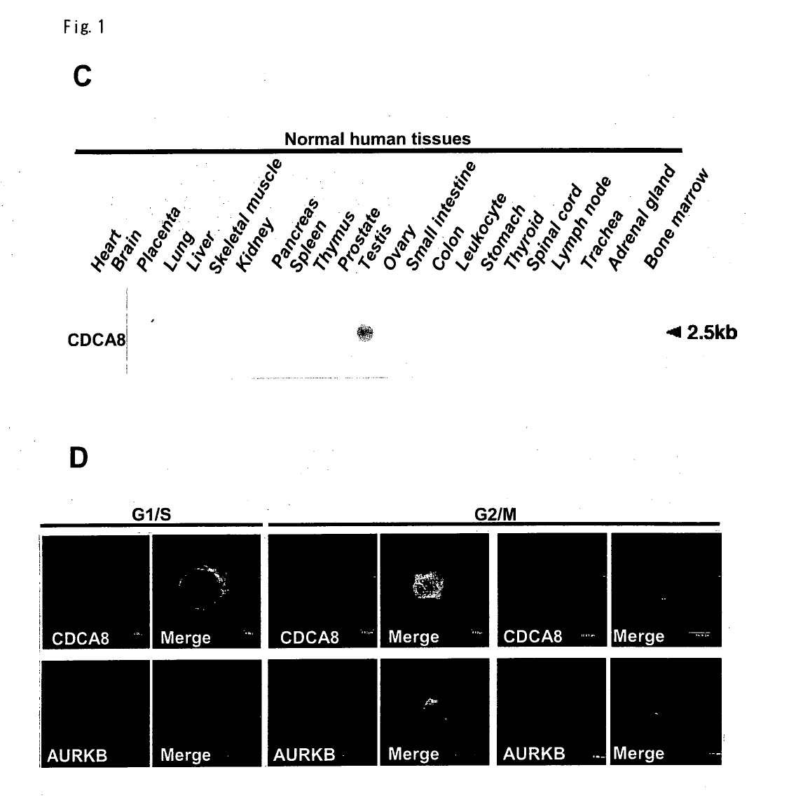

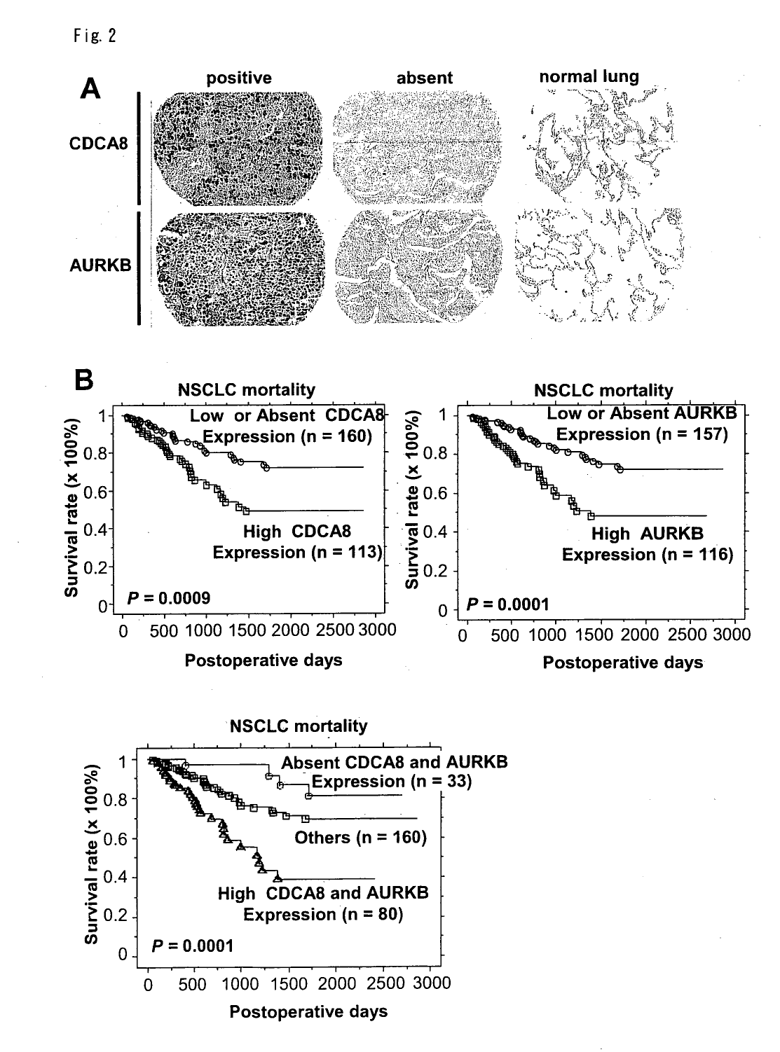

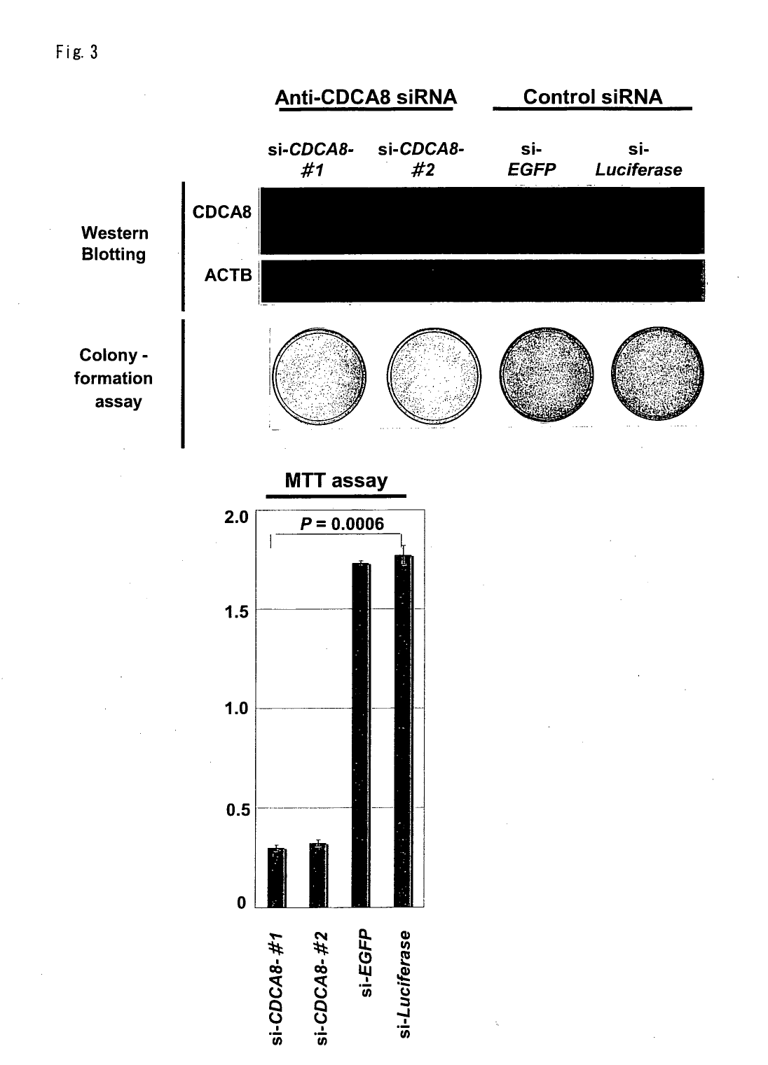

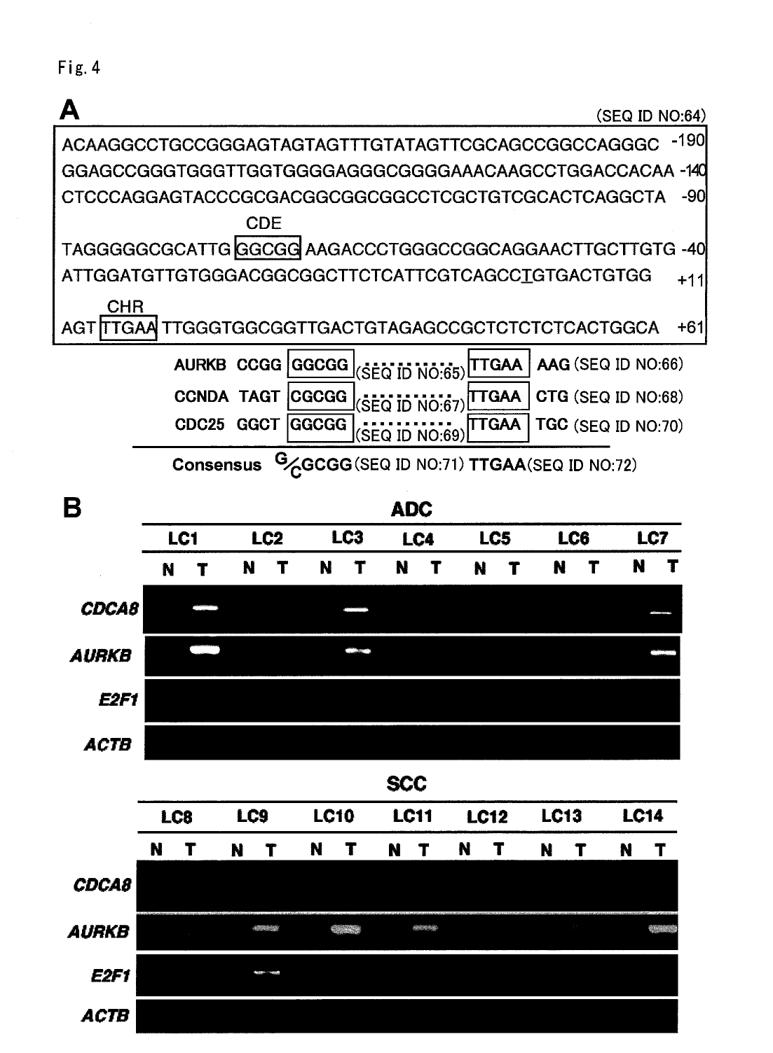

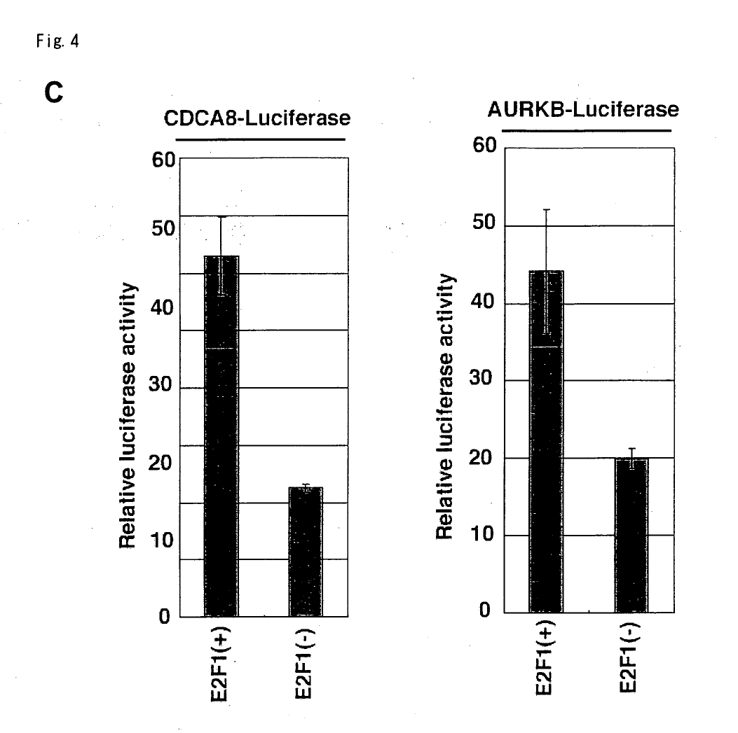

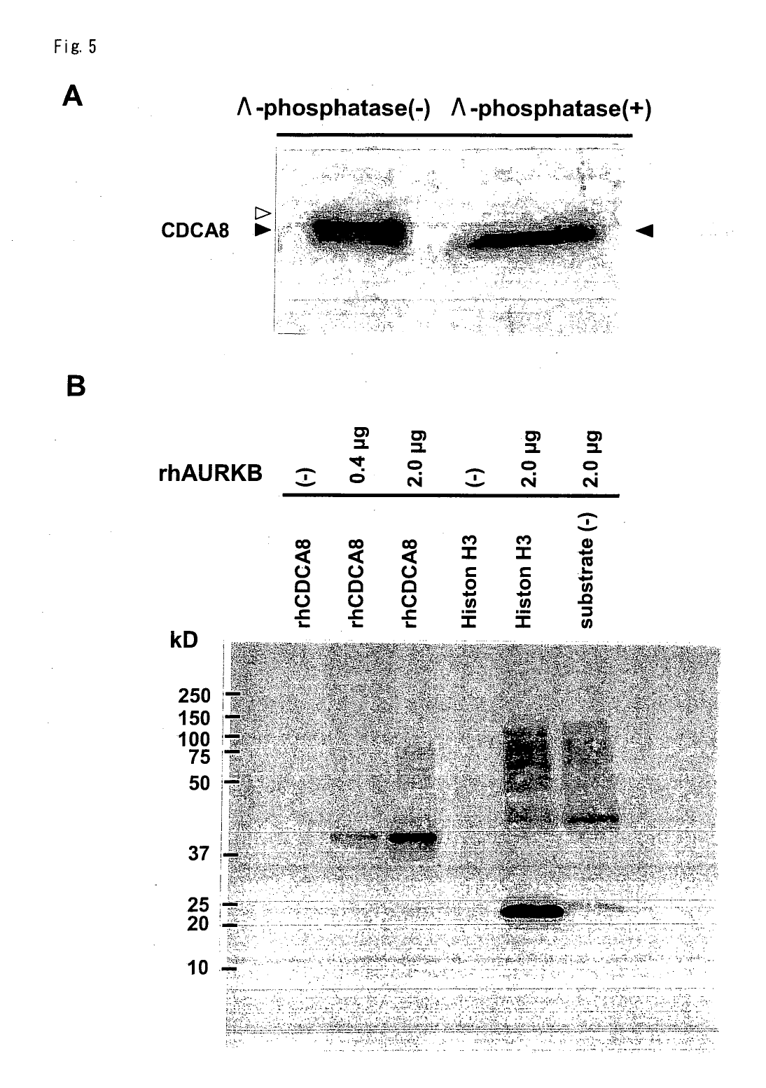

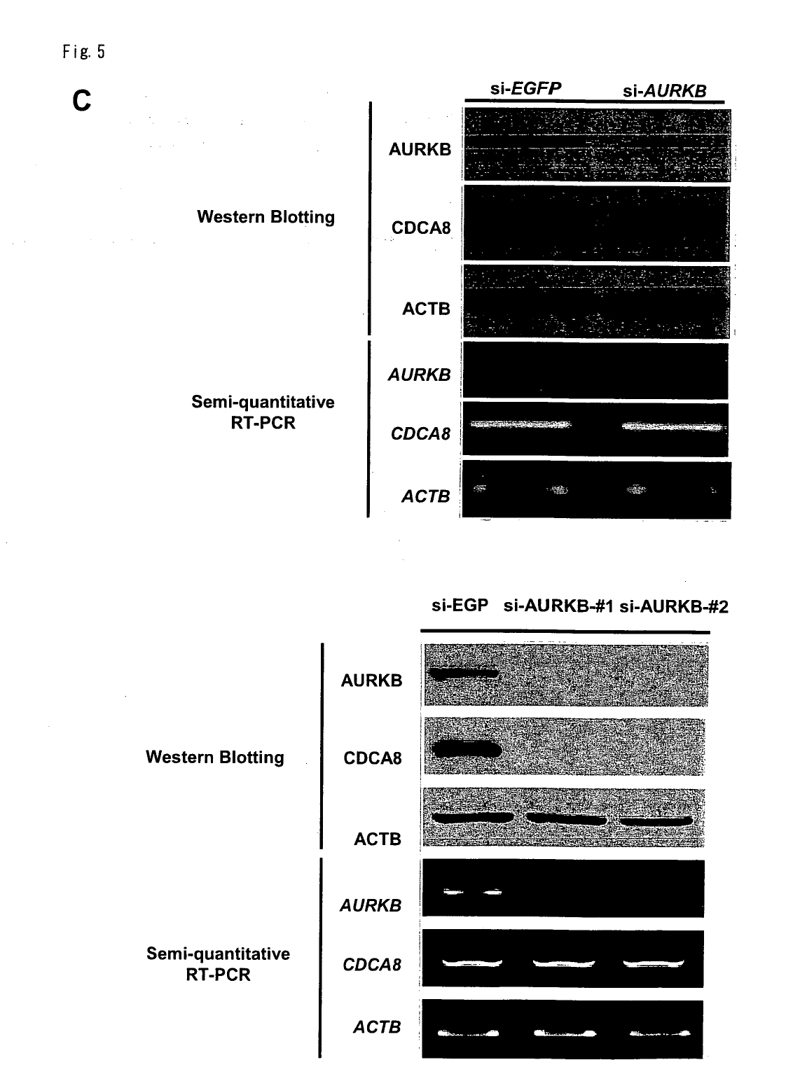

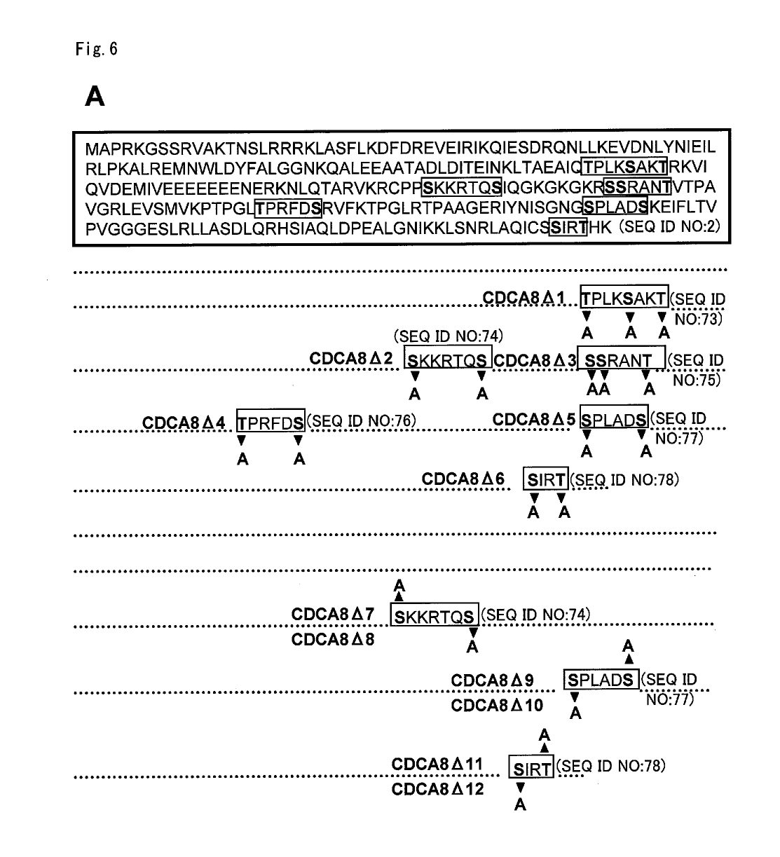

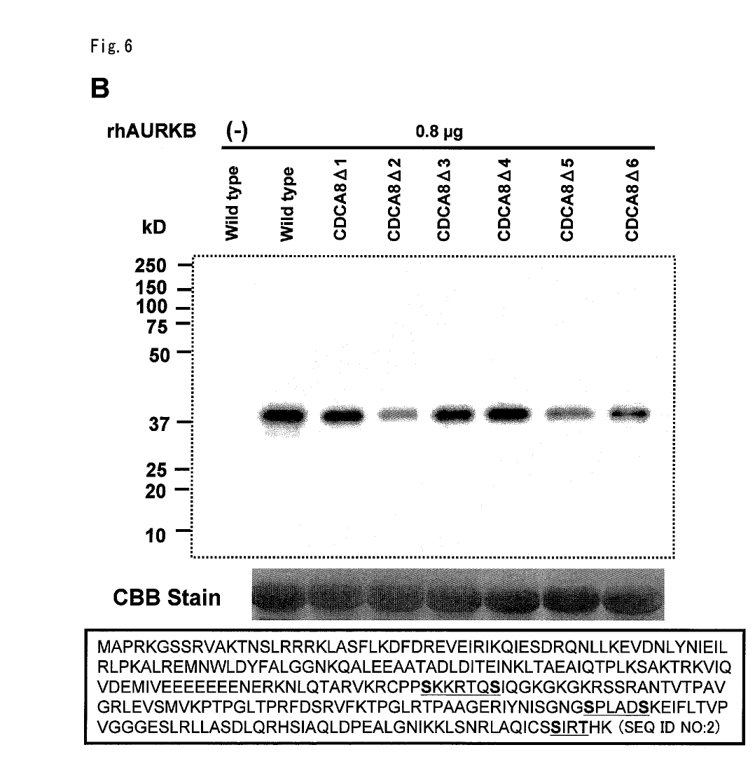

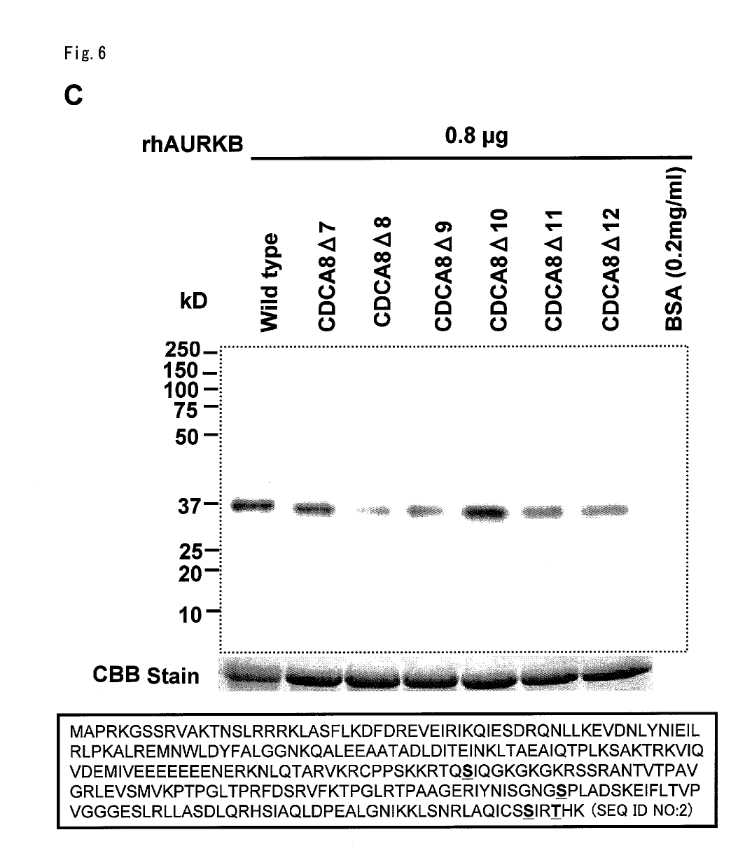

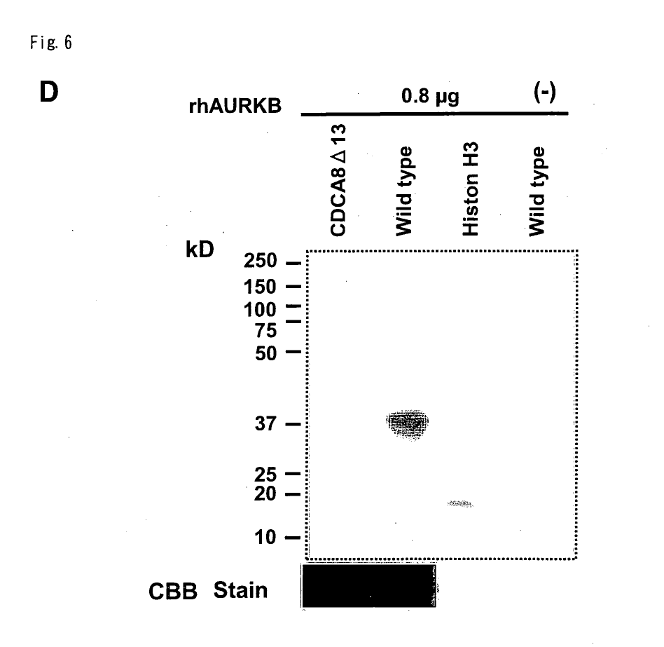

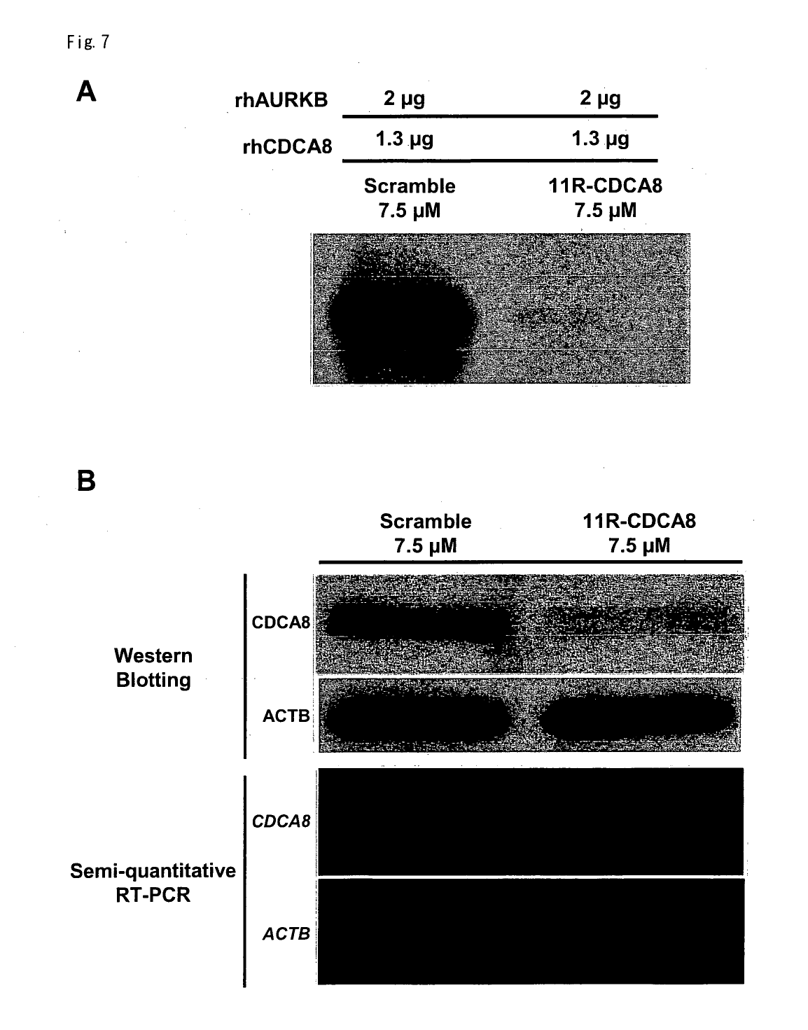

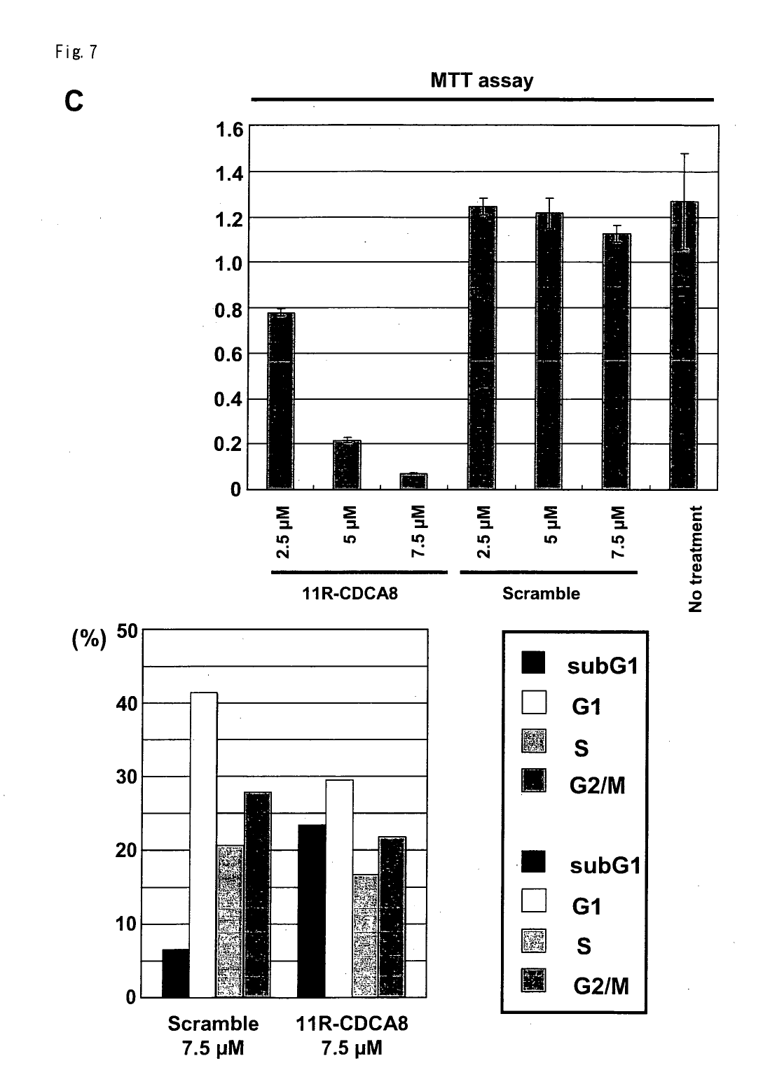

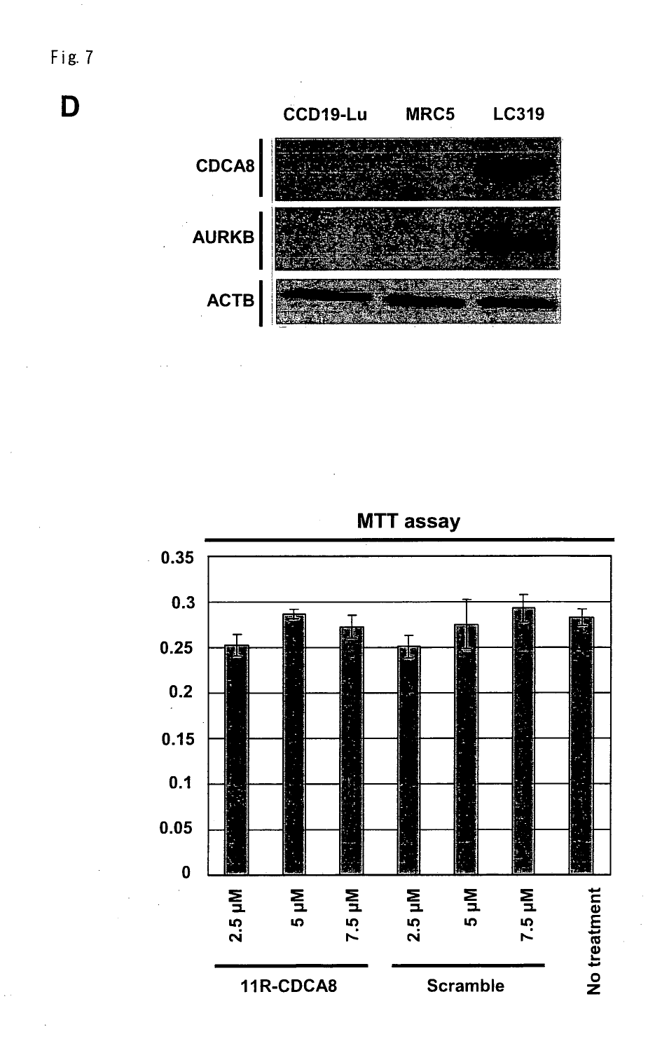

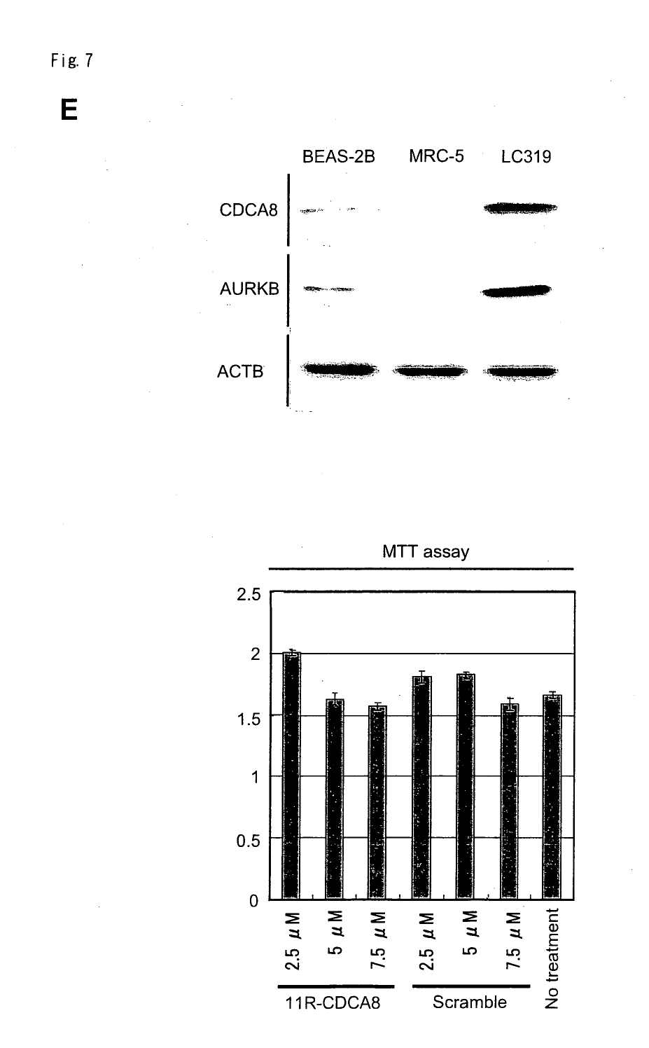

Using a cDNA microarray representing 27,648 genes of 101 lung cancer tissues, CDCA8 was identified as being over-expressed in a large proportion of lung cancers. Its transactivation was subsequently confirmed in 11 of 14 additional NSCLC cases (4 of 7 ADCs; all of 7 SCCs) by semi-quantitative RT-PCR (FIG. 1A, upper panels). High levels of endogenous CDCA8 expression were further confirmed in all of 11 lung-cancer cell lines by western-blot analysis using a rabbit polyclonal anti-CDCA8 antibody (FIG. 1B, upper panel). Northern-blot analysis was performed using CDCA8 cDNA as a probe identified a 2.5-kb transcript, exclusively in the testis among 23 human tissues examined (FIG. 1C).

To determine the subcellular localization of endogenous CDCA8 in lung-cancer cells, immunocytochemical analysis was performed for LC319 cells using anti-CDCA8 antibody; CDCA8 proteins were mainly detected at nucleus in cells at the G1/S phase, and at nucleus and contractile ring in cells at the G2/M phase (FIG. 1D, upper panels). CDCA8 was previously isolated as a new member of a vertebrate chromosomal passenger complex (Sampath S C, et al., Cell. 2004 Jul. 23; 118(2):187-202), and phosphorylation of C-terminal region of GST-tagged CDCA8 by rhAURKB was shown by in vitro kinase assay (Gassmann R, et al., J Cell Biol. 2004 Jul. 19; 166(2):179-91. Epub 2004 Jul. 12).

However, the precise phosphorylation site(s) and its functional significance in cancer cells remain unclear. To elucidate the biological role of CDCA8 activation in lung-cancer cells, the expression status of AURKB was first examined using the previously described gene expression database (Kikuchi T, et al. Oncogene. 2003 Apr. 10; 22(14):2192-205; Kakiuchi S, et al., Mol Cancer Res. 2003 May; 1(7):485-99; Kakiuchi S, et al., Hum Mol Genet. 2004 Dec. 15; 13(24):3029-43. Epub 2004 Oct. 20; Kikuchi T, et al., Int J Oncol. 2006 April; 28(4):799-805; Taniwaki M, et al., Int J Oncol. 2006 September; 29(3):567-75). AURKB was discovered to be frequently over-expressed in lung cancers as compared to normal lung cells. Furthermore, the data herein indicated that the levels of AURKB expression seemed to be correlated with those of CDCA8.

Accordingly, primary lung cancer tissues were subsequently re-examined and an increase in AURKB expression was discovered in 12 of 16 NSCLC clinical samples (5 of 7 ADCs and all of 7 SCCs) examined by semi-quantitative RT-PCR (FIG. 1A, middle panels). The expression patterns of AURKB in lung cancers were very similar to those of CDCA8. It was further confirmed by western-blot analysis that CDCA8 and AURKB proteins were co-activated in almost all of the lung-cancer cell lines examined (FIG. 1B, middle panel). Immunocytochemical analysis using a rabbit polyclonal anti-AURKB antibody detected AURKB proteins to be located mainly at nucleus in cells at the G1/S phase, and at nucleus and contractile ring in cells at the G2/M phase; the subcellular localization of AURKB in lung cancer cells was very similar to that of CDCA8 as reported elsewhere (Sampath S C, et al., Cell. 2004 Jul. 23; 118(2):187-202) (FIG. 1D, right panels).

(b) Association of CDCA8 and AURKB Positivity with Poor Prognosis of NSCLC Patients:

Using tissue microarrays prepared from 273 surgically-resected NSCLCs, immunohistochemical analysis was performed with affinity-purified anti-CDCA8 and anti-AURKB polyclonal antibodies. Patterns of CDCA8/AURKB expression were classified as absent (scored as 0), weak (scored as 1+) or strong (scored as 2+). Of the 273 NSCLC cases examined, 113 cases (41%) revealed strong CDCA8 expression and 100 cases (37%) showed weak expression while its expression was absent in 60 cases (22%). For AURKB, strong expression was observed in 116 cases (42%), weak expression in 94 cases (34%), and no expression in 63 cases (24%). No staining was observed for either CDCA8 or AURKB in any of their adjacent normal lung tissues (FIG. 2A).

183 of the 273 tumors were positive (scored as 1+˜2+) for both. CDCA8 and AURKB, and 33 were negative for the both proteins. 30 of the 273 cases were positive for only CDCA8 and 27 were positive for only AURKB. The expression pattern of CDCA8 protein was significantly concordant with AURKB protein expression in these tumors (P<0.0001 by χ^{2-test}) as similar to the results by RT-PCR and western blotting. Strong expression (scored as 2+) of CDCA8 in NSCLCs was found to be significantly associated with tumor size (pT1 vs pT2-4; P=0.0414 by χ^{2-test}), lymph node metastasis (pN0 vs pN1-4; P=0.0005 by χ^{2-test}), and with shorter tumor-specific 5-year survival times (P=0.0009 by the Log-rank test) (FIG. 2B, upper left panel). Strong expression (scored as 2+) of AURKB in NSCLCs was significantly associated with tumor size (pT1 vs pT2-4; P=0.0361 by χ^{2-test}), lymph node metastasis (pN1 vs pN2-4; P=0.0004 by χ^{2-test}), and 5 year-survival (P=0.0001 by the Log-rank test) (FIG. 2B, upper right panel). NSCLC patients without either CDCA8 or AURKB expression in their tumors could reveal the longest survival period, while those with strong positive staining for both markers showed the shortest tumor-specific survival (P<0.0001 by the Log-rank test; FIG. 2B, lower panel). Using univariate analysis, lymph node metastasis (pN0 vs pN1, N2: P<0.0001; score test), tumor size (pT1 vs pT2, T3, T4: P<0.0001; score test), and high CDCA8/AURKB expression (P=0.0009 and =0.0001, respectively; score test) were discovered to be important correlative features for poor prognoses of patients with NSCLC.

(c) Growth Inhibition of Lung-Cancer Cells by Specific siRNA Against CDCA8:

To assess whether CDCA8 is essential for growth or survival of lung-cancer cells, plasmids were constructed to express siRNA against CDCA8 (si-CDCA8-#1 and si-CDCA8-#2), using siRNAs for EGFP and Luciferase as controls. Transfection of si-CDCA8-#1 or si-CDCA8-#2 into LC319 or SBC-5 cells significantly suppressed expression of endogenous CDCA8 proteins in comparison with the two controls, and resulted in significant decreases in cell viability and colony numbers measured by MTT and colony-formation assays (representative data of LC319 was shown in FIG. 3).

### (d) Simultaneous Activation of CDCA8 and AURKB Regulated by E2F-1:

The concordant activation of CDCA8 and AURKB in lung cancers suggested that these two genes might be regulated by the same transcription factor(s). To validate this hypothesis, the DNA sequences of the CDCA8 and AURKB promoter regions were examined, and found to possess the cell cycle-dependent element (CDE) and cell cycle-gene homology region (CHR) consensus sequences (CDE-CHR), by which transcription of AURKB as well as cyclin A (CCNDA) and CDC25 are regulated (FIG. 4A). Among the transcription factors that could bind to the CDE-CHR of the AURKB gene, it was confirmed that E2F-1 was co-activated with CDCA8 and AURKB as detected by semi-quantitative RT-PCR analysis of NSCLC cases (FIG. 4B).

To investigate the direct transcriptional regulation of the CDCA8 gene promoter by E2F-1, LC319 cells were transiently co-transfected with E2F-1 or mock vector, along with CDCA8 or AURKB (positive control) promoter constructs containing putative regulatory elements (CDE-CHR) fused to a luciferase reporter gene. Expectedly, both CDCA8 and AURKB promoter functions were activated by E2F-1 (FIG. 4C). The induction of endogenous CDCA8 and AURKB was further confirmed by introduction of exogenous E2F-1 into LC319 cells (data not shown).

### (e) Phosphorylation of CDCA8 by AURKB in Lung Cancer Cells:

Western-blot analysis detected two different sizes of CDCA8 protein (FIG. 1B upper panel). To examine a possibility the CDCA8 phosphorylation, extracts from LC319 cells were incubated in the presence or absence of protein phosphatase (BIO-RAD Laboratories, Hercules, Calif.) and analyzed the molecular weight of CDCA8 protein by western-blot analysis. Since the measured weight of the majority of CDCA8 protein in the extracts treated with phosphatase was smaller than that in the untreated cells (FIG. 5A), it was considered that CDCA8 was phosphorylated in lung-cancer cells. The question of whether AURKB could phosphorylate CDCA8 in vitro as reported previously was then examined (Gassmann R, et al., J Cell Biol. 2004 Jul. 19; 166(2):179-91. Epub 2004 Jul. 12).

When full-length recombinant CDCA8 protein was incubated with recombinant AURKB protein in kinase buffer including [gamma-³²P] ATP; CDCA8 was phosphorylated in an AURKB-dose-dependent manner (FIG. 5B). To assess whether the abundant expression of endogenous AURKB is critical for phosphorylation of endogenous CDCA8 in cancer cells, the AURKB expression in LC319 cells, in which these two genes were expressed abundantly was selectively knocked down with siRNA against AURKB (si-AURKB) (FIG. 5C, upper panel).

Reduction of AURKB protein by si-AURKBs (si-AURKB-#1 and -#2) dramatically decreased the amount of CDCA8 protein, while a level of CDCA8 transcripts in the same cells was not influenced by si-AURKB (FIG. 5C, lower panel). Reduction of AURKB protein by si-AURKB decreased the amount of CDCA8 protein as well as phosphorylation levels of CDCA8, while a level of CDCA8 transcripts in the same cells was not influenced by si-AURKBs (FIG. 5C). Hence, it was hypothesized that endogenous CDCA8 protein may be stabilized when it is phosphorylated by endogenous AURKB and/or incorporated in some protein complex.

Since the data herein suggest that human CDCA8 and AURKB were co-activated in lung-cancer cells and that CDCA8 phosphorylation by AURKB might play a significant role in pulmonary carcinogenesis, the phosphorylation sites of CDCA8 by AURKB were then investigated. Six His-tagged CDCA8 proteins (CDCA8delta1-delta6) in which two or three serine/threonine residues were substituted to alanines were prepared (FIG. 6A, upper panel).

In vitro kinase assays were performed using these mutant-CDCA8 proteins, and a reduction of phosphorylation levels was detected in three mutated constructs (CDCA8delta2, -delta5, and -delta6), as compared to a wild-type, suggesting that six serine/threonines corresponding to the substituted sites could be putative phosphorylation ones (FIG. 6B). Six additional mutant-CDCA8 constructs, CDCA8delta7-delta12, in which either of the six serine/threonines were substituted to an alanine were further prepared (FIG. 6A, lower panel). In vitro kinase assays of these six mutants revealed the reduction of phosphorylation levels in four mutated-constructs including a substitution at either of four serine/threonine residues, Ser-154, Ser-219, Ser-275, and Thr-278 (CDCA8delta8, -delta9, -delta11, and -delta12) (FIG. 6C).

A mutant construct (CDCA8delta13), in which all of these four serine/threonines were substituted to an alanine, was subsequently made and in vitro kinase assays were performed using AURKB. The results, showing complete disappearance of CDCA8 phosphorylation by AURKB (FIG. 6D), clearly demonstrated that CDCA8 was phosphorylated at four serine/threonine residues at Ser-154, Ser-219, Ser-275, and Thr-278 by AURKB.

### (f) Growth Inhibition of Lung-Cancer Cells by Cell-Permeable Peptides:

To investigate the functional significance of interaction between CDCA8 and AURKB as well as CDCA8 phosphorylation for growth or survival of lung-cancer cells, bioactive cell-permeable peptides that were expected to inhibit the in vivo phosphorylation of CDCA8 by AURKB were developed. Three different peptides of 19 or 20-amino-acid that included the four CDCA8 phosphorylation sites (Ser-154, Ser-219, Ser-275, and Thr-278) were synthesized. These peptides were covalently linked at its N-terminus to a membrane transducing 11 arginine-residues (11R). The effect of the three 11R-CDCA8 peptides on the phosphorylation of recombinant CDCA8 (rhCDCA8) by recombinant AURKB (rhAURKB) was first investigated using an in vitro kinase assay. When full-length rhCDCA8 protein was incubated with rhAURKB protein with either of the three 11R-CDCA8 peptides or their scramble peptides in kinase buffer including [gamma-³²P] ATP, the phosphorylation level of rhCDCA8 was significantly suppressed by the treatment with 11R-CDCA8₂₆₁₋₂₈₀ peptides containing Ser-154, Ser-219, Ser-275, and Thr-278, compared to its scramble peptides (FIG. 7A).

Addition of the 11R-CDCA8₂₆₁₋₂₈₀ into the culture media of LC319 cells inhibited the phosphorylation and decreased stability of endogenous CDCA8 protein, while no effect on a level of CDCA8 transcript was observed (FIG. 7B). The 11R-CDCA8₂₆₁₋₂₈₀ treatment of LC319 cells resulted in significant decreases in cell viability as measured by MTT assay (representative data in FIG. 7C, upper panel).

To clarify the mechanism of tumor suppression by the 11R-CDCA8₂₆₁₋₂₈₀ peptide, flow cytometric analysis of the tumor cells treated with these peptides was performed, and a significant increase in sub-G1 fraction at 48 hours after the treatment of 11R-CDCA8₂₆₁₋₂₈₀ was discovered (FIG. 7C, lower panel). 11R-CDCA8₂₆₁₋₂₈₀ revealed no effect on cell viability of normal human lung fibroblast derived MRC5, CCD19-Lu cells or human bronchial epithelia derived BEAS-2B cells in which CDCA8 and AURKB expression were hardly detectable (representative data of MRC5 and BEAS-2B cells was shown in FIG. 71) and FIG. 7E). The data indicate that 11R-CDCA8₂₆₁₋₂₈₀ could specifically inhibit an enzymatic reaction of AURKB for CDCA8 phosphorylation, and have no or minimum toxic-effect on normal human cells that do not express these proteins.

### Discussion

With the goal of developing novel therapeutic anti-cancer drugs with a minimum risk of adverse reactions, a powerful screening system to identify proteins and their interacting proteins that were activated specifically in lung cancer cells was established. The strategy was as follows;

1) identify up-regulated genes in 101 lung-cancer samples through the genome-wide cDNA microarray system coupled with laser microdissection (Kikuchi T, et al. Oncogene. 2003 Apr. 10; 22(14):2192-205; Kakiuchi S, et al., Mol Cancer Res. 2003 May; 1(7):485-99; Kakiuchi S, et al., Hum Mol Genet. 2004 Dec. 15; 13(24):3029-43. Epub 2004 Oct. 20; Kikuchi T, et al., Int J Oncol. 2006 April; 28(4):799-805; Taniwaki M, et al., Int J Oncol. 2006 September; 29(3):567-75),

2) verify very low or absent expression of such genes in normal organs by cDNA microarray analysis and multiple-tissue northern blot analysis (Ochi K, et al., J Hum Genet. 2003; 48(4):177-82. Epub 2003 Feb. 21; Adams R R, et al., Trends Cell Biol. 2001 February; 11(2):49-54),

3) confirm the clinicopathological significance of their over-expression using tissue microarray consisting of hundreds of NSCLC tissue samples (Taniwaki M, et al., Int J Oncol. 2006 September; 29(3):567-75; Ishikawa N, et al. Clin Cancer Res. 2004 Dec. 15; 10(24):8363-70; Kato T, et al., Cancer Res. 2005 Jul. 1; 65(13):5638-46; Furukawa C, et al., Cancer Res. 2005 Aug. 15; 65(16):7102-10; Ishikawa N, et al., Cancer Res. 2005 Oct. 15; 65(20):9176-84; Suzuki C, et al., Cancer Res. 2005 Dec. 15; 65(24):11314-25; Ishikawa N, et al., Cancer Sci. 2006 August; 97(8):737-45; Takahashi K, et al., Cancer Res. 2006 Oct. 1; 66(19):9408-19; Hayama S, et al., Cancer Res. 2006 Nov. 1; 66(21):10339-48), and

4) verify whether the targeted genes are essential for the survival or growth of lung cancer cells by siRNA (Suzuki C, et al., Cancer Res. 2003 Nov. 1; 63(21):7038-41; Kato T, et al., Cancer Res. 2005 Jul. 1; 65(13):5638-46; Furukawa C, et al., Cancer Res. 2005 Aug. 15; 65(16):7102-10; Suzuki C, et al., Cancer Res. 2005 Dec. 15; 65(24):11314-25; Ishikawa N, et al., Cancer Sci. 2006 August; 97(8):737-45; Takahashi K, et al., Cancer Res. 2006 Oct. 1; 66(19):9408-19; Hayama S, et al., Cancer Res. 2006 Nov. 1; 66(21):10339-48).

By this systematic approach, CDCA8 and AURKB were identified as being co-overexpressed in the great majority of clinical lung-cancer samples as well as lung cancer cell-lines. Moreover, these two proteins were determined to be indispensable for growth and progression of lung cancer cells.

CDCA8 was shown to be phosphorylated in vitro by AURKB previously (Gassmann R, et al., J Cell Biol. 2004 Jul. 19; 166(2):179-91. Epub 2004 Jul. 12); however, its significance in development and/or progression of human cancer has not previously been described. CDCA8 was recently indicated to be one of new components of the vertebrate chromosomal passengers, such as AURKB, INCENP, and BIRC5 (Sampath S C, et al., Cell. 2004 Jul. 23; 118(2):187-202), which are considered to be key regulators of mitotic events responsible for correcting the error of bipolar attachments that inevitably occur during the ‘search-and-capture’ mechanism (Adams R R, et al., J Cell Biol. 2001 May 14; 153(4):865-80; Wheatley S P, et al., Curr Biol. 2001 Jun. 5; 11(11):886-90; Walker M G. Curr Cancer Drug Targets. 2001 May; 1(1):73-83).

Herein it was demonstrated that the CDCA8 protein is likely to be stabilized by its AURKB-dependent phosphorylation at Ser-154, Ser-219, Ser-275, and/or Thr-278. Depletion of AURKB function by RNA interference or the 11R-CDCA8₂₆₁₋₂₈₀ that could inhibit phosphorylation of CDCA8 significantly decreased the level of endogenous CDCA8 protein. Phosphorylation is an important post-translational modification that regulates the protein stability, function, localization, and binding-specificity to target proteins. For example, MKP-7 phosphorylated at Ser-446 or p27 phosphorylated at Ser-10 have a longer half-life than unphosphorylated form; when at the sites were dephosphorylated, the amount of these proteins was promptly decreased in cells (Katagiri C, et al. J Biol Chem 2005; 280:14716-22; Deng X, et al. J Biol Chem 2004; 279:22498-504).

The evidence herein suggests that the stability of CDCA8 protein could be tightly regulated by the AURKB signaling in cancel cells. AURKB was shown to be over-expressed in many tumor cell lines, and its over-expression was noted to be involved in chromosome number instability and tumor invasiveness (Bischoff J R, et al., EMBO J. 1998 Jun. 1; 17(11):3052-65; Branca M, et al., Am J Clin Pathol. 2005 July; 124(1):113-21). AURKB is one of the cancer-related kinases, and therefore was thought to be a promising target for anticancer drug development. Indeed two AURKB inhibitors have recently been described: ZM447439 and Hesperadin (Keen N & Taylor S. Nat Rev Cancer. 2004 December; 4(12):927-36). The results herein indicate that CDCA8 is a putative oncogene that is aberrantly expressed in lung cancer cells along with AURKB.

As the results of tissue microarray analysis discussed above demonstrate, CDCA8 and AURKB are co-overexpressed, and patients with NSCLC showing higher expression of these proteins represent a shorter tumor-specific survival period, both of which doubtlessly suggest that CDCA8, along with AURKB, plays a crucial role for progression of lung cancers.

Also demonstrated herein for the first time is the fact that growth of lung-cancer cells over-expressing CDCA8 and AURKB can be suppressed effectively by blocking the AURKB-dependent CDCA8 phosphorylation by means of the 20 amino-acid cell-permeable peptide that corresponds to a part of CDCA8 protein and includes two phosphorylation sites, Ser-154, Ser-219, Ser-275, and Thr-278, by AURKB. A significant increase in the sub-G1 fraction was detected after the treatment of 11R-CDCA8₂₆₁₋₂₈₀ peptide, suggesting that the cell-permeable polypeptides induced apoptosis of the cancer cells. Since the phosphorylation of CDCA8 at these sites are likely to be indispensable for the growth/survival of lung cancer cells, and CDCA8 can belong to cancer-testis antigens, selective targeting of CDCA8-AURKB enzymatic activity constitutes a promising therapeutic strategy that is expected to have a powerful biological activity against cancer with a minimal risk of adverse events. Further analyses of the mechanism of growth suppression by specific inhibiting of CDCA8-phosphorylation by AURKB may be of the great benefit towards the development of new types of anti-cancer agents.

In summary, the present invention relates to the discovery that CDCA8 is co-activated with and phosphorylated/stabilized by AURKB in lung cancer cells, and that phosphorylated CDCA8 plays a significant role in growth and/or survival of cancer cells. The data herein strongly suggest that new anti-cancer drugs designed to target the CDCA8-AURKB association constitute a promising therapeutic strategy for lung cancer.

### INDUSTRIAL APPLICABILITY

As demonstrated herein, the coactivation of CDCA8 and A URKB, and their cognate interactions, play a significant role in lung cancer progression. Thus, agents that directly or indirectly inhibit the formation of this complex find therapeutic utility as anti-cancer agents for the treatment of lung cancer, more particularly NSCLC.

In addition, the present invention provides screening methods for anti-cancer agents that directly or indirectly inhibit the formation of the CDCA8-AURKB complex, for example by inhibiting the binding between CDCA8 and AURKB, inhibiting or reducing the phosphorylation of CDCA8 by AURKB, or by inhibiting or suppressing the expression of CDCA8, AURKB, or both. CDCA8 has been shown to be upregulated in non-small-cell lung cancer. Moreover, as CDCA8 antisense nucleotides are shown herein to inhibit cell growth, more particularly inhibit the proliferation of NSCLC cells, it is expected that candidate compounds that inhibit the formation of the CDCA8-AURKB complex will also serve to inhibit NSCLC cell proliferation. The present invention further provides diagnostic and prognostic methods that utilize expression levels of CDCA8 and/or AURKB as a determining index.

All patents, patent applications, and publications cited herein are incorporated by reference in their entirety. However, nothing herein should be construed as an admission that the invention is not entitled to antedate such disclosure by virtue of prior invention.

While the present invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Further advantages and features will become apparent from the claims filed hereafter, with the scope of such claims to be determined by their reasonable equivalents, as would be understood by those skilled in the art. Thus, the invention is intended to be defined not by the above description, but by the following claims and their equivalents.

1. A method of screening for a candidate compound for treating or preventing non-small cell lung cancer (NSCLC), said method comprising the steps of:
(a) contacting an AURKB polypeptide or functional equivalent thereof with a CDCA8 polypeptide or functional equivalent thereof in the presence of a test compound;
(b) assaying the binding between the polypeptides of step (a); and
(c) selecting the test compound that inhibits the binding between the AURKB and CDCA8 polypeptides.

2. The method of claim 1, wherein the functional equivalent of the CDCA8 polypeptide comprises the amino acid sequence of the AURKB binding domain.

3. The method of claim 2, wherein the functional equivalent of the CDCA8 polypeptide comprises the amino acid sequence of SEQ ID NO: 5 (NIKKLSNRLAQICSSIRTHK).

4. The method of claim 1, wherein the functional equivalent of the AURKB polypeptide comprises the amino acid sequence of the CDCA8 binding domain.

5. A kit for screening for a compound for treating or preventing NSCLC, said kit comprising the components of:
(a) an AURKB polypeptide or functional equivalent thereof, and
(b) a CDCA8 polypeptide or functional equivalent thereof.

6. A method of screening for a candidate compound for treating or preventing NSCLC, said method comprising the steps of:
(a) incubating CDCA8 and AURKB in the presence of a test compound under conditions suitable for the phosphorylation of CDCA8 by AURKB, wherein the CDCA8 is a polypeptide selected from the group consisting of:
i. a polypeptide comprising the amino acid sequence of SEQ ID NO: 2 (CDCA8);
ii. a polypeptide comprising the amino acid sequence of SEQ ID NO: 2 wherein one or more amino acids are substituted, deleted, or inserted, provided said polypeptide has a biological activity equivalent to the polypeptide consisting of the amino acid sequence of SEQ ID NO: 2;
iii. a polypeptide encoded by a polynucleotide that hybridizes under stringent conditions to a polynucleotide consisting of the nucleotide sequence of SEQ ID NO: 1, provided the polypeptide has a biological activity equivalent to a polypeptide consisting of the amino acid sequence of SEQ ID NO: 2; and
wherein the AURKB is a polypeptide selected from the group consisting of:
i. a polypeptide the amino acid sequence of SEQ ID NO: 4 (AURKB);
ii. a polypeptide having the amino acid sequence of SEQ ID NO: 4 wherein one or more amino acids are substituted, deleted, or inserted, provided the polypeptide has a biological activity equivalent to the polypeptide consisting of the amino acid sequence of SEQ ID NO: 4;
iii. a polypeptide encoded by a polynucleotide that hybridizes under stringent conditions to a polynucleotide consisting of the nucleotide sequence of SEQ ID NO: 3, provided the polypeptide has a biological activity equivalent to a polypeptide consisting of the amino acid sequence of SEQ ID NO: 4;
(b) detecting a phosphorylation level of the CDCA8;
(c) comparing the phosphorylation level of the CDCA8 measured in step (b) to a control level; and
(d) selecting a compound that decreases the phosphorylation level of the CDCA8 as compared to the control level.

7. The method of claim 6, wherein the phosphorylation level of the CDCA8 is detected at one or more phosphorylation site selected from the group consisting of Ser-154, Ser-219, Ser-275, and Thr-278 of the amino acid sequence of SEQ ID NO: 2, or homologous positions of the polypeptide.

8. A kit for screening for a candidate compound for treating or preventing NSCLC, said kit comprising the components of:
(a) a polypeptide selected from the group consisting of:
i. a polypeptide comprising the amino acid sequence of SEQ ID NO: 2 (CDCA8);
ii. a polypeptide comprising the amino acid sequence of SEQ ID NO: 2 wherein one or more amino acids are substituted, deleted, or inserted, provided said polypeptide has a biological activity equivalent to the polypeptide consisting of the amino acid sequence of SEQ ID NO: 2; and
iii. a polypeptide encoded by a polynucleotide that hybridizes under stringent conditions to a polynucleotide consisting of the nucleotide sequence of SEQ ID NO: 1, provided the polypeptide has a biological activity equivalent to a polypeptide consisting of the amino acid sequence of SEQ ID NO: 2;
(b) a polypeptide selected from the group consisting of:
i. a polypeptide comprising the amino acid sequence of SEQ ID NO: 4 (AURKB);
ii. a polypeptide comprising the amino acid sequence of SEQ ID NO: 4 wherein one or more amino acids are substituted, deleted, or inserted, provided said polypeptide has a biological activity equivalent to the polypeptide consisting of the amino acid sequence of SEQ ID NO: 4; and
iii. a polypeptide encoded by a polynucleotide that hybridizes under stringent conditions to a polynucleotide consisting of the nucleotide sequence of SEQ ID NO: 3, provided the polypeptide has a biological activity equivalent to a polypeptide consisting of the amino acid sequence of SEQ ID NO: 4; and
(c) a reagent for detecting a phosphorylation level of CDCA8.

9. A kit for screening for a candidate compound for treating or preventing NSCLC, said kit comprising the components of:
(a) a cell expressing a polypeptide selected from the group consisting of:
i. a polypeptide comprising the amino acid sequence of SEQ ID NO: 2 (CDCA8);
ii. a polypeptide comprising the amino acid sequence of SEQ ID NO: 2 wherein one or more amino acids are substituted, deleted, or inserted, provided said polypeptide has a biological activity equivalent to the polypeptide consisting of the amino acid sequence of SEQ ID NO: 2;
iii. a polypeptide encoded by a polynucleotide that hybridizes under stringent conditions to a polynucleotide consisting of the nucleotide sequence of SEQ ID NO: 1, provided the polypeptide has a biological activity equivalent to a polypeptide consisting of the amino acid sequence of SEQ ID NO: 2; and
(b) a reagent for detecting a phosphorylation level of CDCA8.

10. The kit of claim 9, wherein the cell further expresses a polypeptide selected from the group consisting of:
i. a polypeptide comprising the amino acid sequence of SEQ ID NO: 4 (AURKB);
ii. a polypeptide comprising the amino acid sequence of SEQ ID NO: 4 wherein one or more amino acids are substituted, deleted, or inserted, provided said polypeptide has a biological activity equivalent to the polypeptide consisting of the amino acid sequence of SEQ ID NO: 4; and
iii. a polypeptide encoded by a polynucleotide that hybridizes under stringent conditions to a polynucleotide consisting of the nucleotide sequence of SEQ ID NO: 3, provided the polypeptide has a biological activity equivalent to a polypeptide consisting of the amino acid sequence of SEQ ID NO: 4.

11. The kit of claim 9, wherein the cell is NSCLC cell.

12. The kit of claim 8 or 9, wherein the reagent for detecting a phosphorylation level of CDCA8 is an antibody that recognizes the phosphorylation at any one of phosphorylation site selected from the group consisting of Ser-154, Ser-219, Ser-275, and Thr-278 of the amino acid sequence of SEQ ID NO: 2.

13-40. (canceled)

41. A method of assessing an NSCLC prognosis, wherein the method comprises the steps of:
(a) detecting the expression level of either of CDCA8 and AURKB, or both in a specimen collected from a subject whose NSCLC prognosis is to be assessed, and
(b) indicating a poor prognosis when an elevation in the expression level of either of CDCA8 and AURKB, or both is detected.

42. The method of claim 41, wherein the expression level is detected by any one of the methods selected from the group consisting of:
(a) detecting the presence of an mRNA encoding the amino acid sequence of SEQ ID NO: 2 (CDCA8) or SEQ ID NO: 4 (AURKB),
(b) detecting the presence of a protein comprising the amino acid sequence of SEQ ID NO: 2 (CDCA8) or SEQ ID NO: 4 (AURKB), and
(c) detecting the biological activity of a protein comprising the amino acid sequence of SEQ ID NO: 2 (CDCA8) or SEQ ID NO: 4 (AURKB).

43. A kit for assessing an NSCLC prognosis, wherein the kit comprises any one component selected from the group consisting of:
(a) a reagent for detecting the presence of an mRNA encoding the amino acid sequence of SEQ ID NO: 2 (CDCA8) or SEQ ID NO: 4 (AURKB),
(b) a reagent for detecting the presence of a protein comprising the amino acid sequence of SEQ ID NO: 2 (CDCA8) or SEQ ID NO: 4 (AURKB), and
(c) a reagent for detecting the biological activity of a protein comprising the amino acid sequence of SEQ ID NO: 2 (CDCA8) or SEQ ID NO: 4 (AURKB).

44-53. (canceled)


SEQUENCE LISTING (63 sequences)

SEQ ID NO: 1 - DNA, 2319 bp, Homo sapiens
  CDS (108)..(947)
gtggagtttgaattgggtggcggttgactgtagagccgctctctctcactggcacagcgaggttttgctcagcccttgtctcgggaccgcagcctccgccgagcgccatggctcctaggaagggcagtagtcgggtggccaagaccaactccttacggaggcggaagctcgcctcctttctgaaagacttcgaccgtgaagtggaaatacgaatcaagcaaattgagtcagacaggcagaacctcctcaaggaggtggataacctctacaacatcgagatcctgcggctccccaaggctctgcgcgagatgaactggcttgactacttcgcccttggaggaaacaaacaggccctggaagaggcggcaacagctgacctggatatcaccgaaataaacaaactaacagcagaagctattcagacacccctgaaatctgccaaaacacgaaaggtaatacaggtagatgaaatgatagtggaagaggaagaagaagaagaaaatgaacgtaagaatcttcaaactgcaagagtcaaaaggtgtcctccatccaagaagagaactcagtccatacaaggaaaaggaaaagggaaaaggtcaagccgtgctaacactgttaccccagccgtgggccgattggaggtgtccatggtcaaaccaactccaggcctgacacccaggtttgactcaagggtcttcaagacccctggcctgcgtactccagcagcaggagagcggatttacaacatctcagggaatggcagccctcttgctgacagcaaagagatcttcctcactgtgccagtgggcggcggagagagcctgcgattattggccagtgacttgcagaggcacagtattgcccagctggatccagaggccttgggaaacattaagaagctctccaaccgtctcgcccaaatctgcagcagcatacggacccacaaatgagacaccaaagttgacaggatggacttttaatgggcacttctgggaccctgaagagacttcttcccttcaggcttattgtttgagtgtgaagttccagagcaaggagccatgttcctctaagggaattcaggaattcagacgtgctagtcccacaccagttaggtagagctgtctgttcaccctcccatcccagctgatcccagtcactgcttgctggggccatgccatggaagcttcccatcagtctcccagctgaatcctccctgctctctgagctgctgccttttgcctcctgcaactcaacatcctcttcaccctgccctgcctgcagttgagggggcgaagaagaaccctgtgttctcaggaagactgcctccaccaccgctacccagagaacctctgcatctggcatttctgctctctatgcttgagaccgggaggtttaggctcagataagtgagctctgggccatgagagggtaggtccagaaggtggggggaactgtacagatcagcagagcaggacagttggcagcagtgacctcagtagggaacatgtccgtctaccctctcgcactcatgacacctccccctaccagccctcctcttcctcctcctcctcctcctgtgggaggtggtcagtgggacttagggatctttcacctgctgtgcccagtagttctgaagtctgcttgtggagcagtgttttatgtttatccctgtttactgaagaccaaatactggtttggagacaacttccatgtcttgctcttctacctccctagttagtggaaatttggataagggaactgtagggcccagattctggaggttttatgtcattggccacagaataactgtctctaagctatccatggtccagtggtccctgccaagtctgtagacttcagagagcacttctctcttatggggttcatgggaacaggggtgggtgtgacttgcttggtggcctcattccatgtgtgcctgtgcctggggcatggactttgttaagcagagtcagcagtgaggtcctcattctccagccagcctctctgccctggagaatcatgtgctatgttctaagaatttgagaactagagtcctcatccccaggcttgaaggcacatggctttctcatgtagggctctctgtggtatttgttattattttgcaacaagaccattttagtaaaacagtcctgttcaagttgtattcttttaagttcttttattctcctttccctgagatttttgtatatattgttctgagtaatggtatctttgagctgattgttctaatcagagctggtacctactttcaataaattctggttttgtgttttcttttgtaaaaaaaaaaaaaaaaaa

SEQ ID NO: 2 - protein, 280 aa, Homo sapiens
MAPRKGSSRVAKTNSLRRRKLASFLKDFDREVEIRIKQIESDRQNLLKEVDNLYNIEILRLPKALREMNWLDYFALGGNKQALEEAATADLDITEINKLTAEAIQTPLKSAKTRKVIQVDEMIVEEEEEEENERKNLQTARVKRCPPSKKRTQSIQGKGKGKRSSRANTVTPAVGRLEVSMVKPTPGLTPRFDSRVFKTPGLRTPAAGERIYNISGNGSPLADSKEIFLTVPVGGGESLRLLASDLQRHSIAQLDPEALGNIKKLSNRLAQICSSIRTHK

SEQ ID NO: 3 - DNA, 2453 bp, Homo sapiens
gtggagtttgaattgggtggcggttgactgtagagccgctctctctcactggcacagcgaggttttgctcagcccttgtctcgggaccgcagcctccgccgagcgccatggctcctaggaagggcagtagtcgggtggccaagaccaactccttacggaggcggaagctcgcctcctttctgaaagacttcgaccgtgaagtggaaatacgaatcaagcaaattgagtcagacaggcagaacctcctcaaggaggtggataacctctacaacatcgagatcctgcggctccccaaggctctgcgcgagatgaactggcttgactacttcgcccttggaggaaacaaacaggccctggaagaggcggcaacagctgacctggatatcaccgaaataaacaaactaacagcagaagctattcagacacccctgaaatctgccaaaacacgaaaggtaatacaggtagatgaaatgatagtggaagaggaagaagaagaagaaaatgaacgtaagaatcttcaaactgcaagagtcaaaaggtgtcctccatccaagaagagaactcagtccatacaaggaaaaggaaaagggaaaaggtcaagccgtgctaacactgttaccccagccgtgggccgattggaggtgtccatggtcaaaccaactccaggcctgacacccaggtttgactcaagggtcttcaagacccctggcctgcgtactccagcagcaggagagcggatttacaacatctcagggaatggcagccctcttgctgacagcaaagagatcttcctcactgtgccagtgggcggcggagagagcctgcgattattggccagtgacttgcagaggcacagtattgcccagctggatccagaggccttgggaaacattaagaagctctccaaccgtctcgcccaaatctgcagcagcatacggacccacaaatgagacaccaaagttgacaggatggacttttaatgggcacttctgggaccctgaagagacttcttcccttcaggcttattgtttgagtgtgaagttccagagcaaggagccatgttcctctaagggaattcaggaattcagacgtgctagtcccacaccagttaggtagagctgtctgttcaccctcccatcccagctgatcccagtcactgcttgctggggccatgccatggaagcttcccatcagtctcccgggcggccgggagagtagcagtgccttggaccccagctctcctccccctttctctctaaggatggcccagaaggagaactcctacccctggccctacggccgacagacggctccatctggcctgagcaccctgccccagcgagtcctccggaaagagcctgtcaccccatctgcacttgtcctcatgagccgctccaatgtccagcccacagctgcccctggccagaaggtgatggagaatagcagtgggacacccgacatcttaacgcggcacttcacaattgatgactttgagattgggcgtcctctgggcaaaggcaagtttggaaacgtgtacttggctcgggagaagaaaagccatttcatcgtggcgctcaaggtcctcttcaagtcccagatagagaaggagggcgtggagcatcagctgcgcagagagatcgaaatccaggcccacctgcaccatcccaacatcctgcgtctctacaactatttttatgaccggaggaggatctacttgattctagagtatgccccccgcggggagctctacaaggagctgcagaagagctgcacatttgacgagcagcgaacagccacgatcatggaggagttggcagatgctctaatgtactgccatgggaagaaggtgattcacagagacataaagccagaaaatctgctcttagggctcaagggagagctgaagattgctgacttcggctggtctgtgcatgcgccctccctgaggaggaagacaatgtgtggcaccctggactacctgcccccagagatgattgaggggcgcatgcacaatgagaaggtggatctgtggtgcattggagtgctttgctatgagctgctggtggggaacccaccctttgagagtgcatcacacaacgagacctatcgccgcatcgtcaaggtggacctaaagttccccgcttccgtgcccatgggagcccaggacctcatctccaaactgctcaggcataacccctcggaacggctgcccctggcccaggtctcagcccacccttgggtccgggccaactctcggagggtgctgcctccctctgcccttcaatctgtcgcctgatggtccctgtcattcactcgggtgcgtgtgtttgtatgtctgtgtatgtataggggaaagaagggatccctaactgttcccttatctgttttctacctcctcctttgtttaataaaggctgaagctttttgtactcatgaaaaaaaaaaaaaaaaaa

SEQ ID NO: 4 - protein, 344 aa, Homo sapiens
MAQKENSYPWPYGRQTAPSGLSTLPQRVLRKEPVTPSALVLMSRSNVQPTAAPGQKVMENSSGTPDILTRHFTIDDFEIGRPLGKGKFGNVYLAREKKSHFIVALKVLFKSQIEKEGVEHQLRREIEIQAHLHHPNILRLYNYFYDRRRIYLILEYAPRGELYKELQKSCTFDEQRTATIMEELADALMYCHGKKVIHRDIKPENLLLGLKGELKIADFGWSVHAPSLRRKTMCGTLDYLPPEMIEGRMHNEKVDLWCIGVLCYELLVGNPPFESASHNETYRRIVKVDLKFPASVPMGAQDLISKLLRHNPSERLPLAQVSAHPWVRANSRRVLPPSALQSVA

SEQ ID NO: 5 - protein, 20 aa, Artificial
  An artificially synthesised dominant negative peptide
NIKKLSNRLAQICSSIRTHK

SEQ ID NO: 6 - protein, 9 aa, Artificial
  An artificially synthesised Tat sequence
RKKRRQRRR

SEQ ID NO: 7 - protein, 16 aa, Artificial
  An artificially synthesised Penetratin sequence
RQIKIWFQNRRMKWKK

SEQ ID NO: 8 - protein, 21 aa, Artificial
  An artificially synthesised Buforin II sequence
TRSSRAGLQFPVGRVHRLLRK

SEQ ID NO: 9 - protein, 27 aa, Artificial
  An artificially synthesised Transportan sequence
GWTLNSAGYLLGKINLKALAALAKKIL

SEQ ID NO: 10 - protein, 18 aa, Artificial
  An artificially synthesised MAP (model amphipathic peptide) sequence
KLALKLALKALKAALKLA

SEQ ID NO: 11 - protein, 16 aa, Artificial
  An artificially synthesised K-FGF sequence
AAVALLPAVLLALLAP

SEQ ID NO: 12 - protein, 5 aa, Artificial
  An artificially synthesised Ku70 sequence
VPMLK

SEQ ID NO: 13 - protein, 5 aa, Artificial
  An artificially synthesised Ku70 sequence
PMLKE

SEQ ID NO: 14 - protein, 28 aa, Artificial
  An artificially synthesised Prion sequence
MANLGYWLLALFVTMWTDVGLCKKRPKP

SEQ ID NO: 15 - protein, 18 aa, Artificial
  An artificially synthesised pVEC sequence
LLIILRRRIRKQAHAHSK

SEQ ID NO: 16 - protein, 21 aa, Artificial
  An artificially synthesised Pep-1 sequence
KETWWETWWTEWSQPKKKRKV

SEQ ID NO: 17 - protein, 18 aa, Artificial
  An artificially synthesised SynB1 sequence
RGGRLSYSRRRFSTSTGR

SEQ ID NO: 18 - protein, 15 aa, Artificial
  An artificially synthesised Pep-7 sequence
SDLWEMMMVSLACQY

SEQ ID NO: 19 - protein, 12 aa, Artificial
  An artificially synthesised HN-1 sequence
TSPLNIHNGQKL

SEQ ID NO: 20 - protein, 11 aa, Artificial
  An artificially synthesised poly-arginine sequence
RRRRRRRRRRR

SEQ ID NO: 21 - DNA, 23 bp, Artificial
  An artificially synthesized primer sequence for RT-PCR
catctggcatttctgctctctat

SEQ ID NO: 22 - DNA, 25 bp, Artificial
  An artificially synthesized primer sequence for RT-PCR
ctcagggaaaggagaataaaagaac

SEQ ID NO: 23 - DNA, 20 bp, Artificial
  An artificially synthesized primer sequence for RT-PCR
cccatctgcacttgtcctca

SEQ ID NO: 24 - DNA, 24 bp, Artificial
  An artificially synthesized primer sequence for RT-PCR
aacagataagggaacagttaggga

SEQ ID NO: 25 - DNA, 23 bp, Artificial
  An artificially synthesized primer sequence for RT-PCR
ggagtctgtgtggtgtgtatgtg

SEQ ID NO: 26 - DNA, 23 bp, Artificial
  An artificially synthesized primer sequence for RT-PCR
gagggaacagagctgttaggaag

SEQ ID NO: 27 - DNA, 21 bp, Artificial
  An artificially synthesized primer sequence for RT-PCR
gaggtgatagcattgctttcg

SEQ ID NO: 28 - DNA, 21 bp, Artificial
  An artificially synthesized primer sequence for RT-PCR
caagtcagtgtacaggtaagc

SEQ ID NO: 29 - DNA, 19 bp, Artificial
  An artificially synthesized target sequence for siRNA
gaagcagcacgacttcttc

SEQ ID NO: 30 - DNA, 19 bp, Artificial
  An artificially synthesized target sequence for siRNA
cgtacgcggaatacttcga

SEQ ID NO: 31 - DNA, 19 bp, Artificial
  A target sequence for siRNA
cagcagaagctattcagac

SEQ ID NO: 32 - DNA, 19 bp, Artificial
  A target sequence for siRNA
gccgtgctaacactgttac

SEQ ID NO: 33 - DNA, 19 bp, Artificial
  A target sequence for siRNA
ggtgattcacagagacata

SEQ ID NO: 34 - DNA, 51 bp, Artificial
  An artificially synthesized oligonucleotide for siRNA
tcccgaagcagcacgacttcttcttcaagagagaagaagtcgtgctgcttc

SEQ ID NO: 35 - DNA, 51 bp, Artificial
  An artificially synthesized oligonucleotide for siRNA
aaaagaagcagcacgacttcttctctcttgaagaagaagtcgtgctgcttc

SEQ ID NO: 36 - DNA, 47 bp, Artificial
  siRNA hairpin design
gaagcagcacgacttcttcttcaagagagaagaagtcgtgctgcttc

SEQ ID NO: 37 - DNA, 51 bp, Artificial
  Artificially synthesized oligonucleotide for siRNA
tccccgtacgcggaatacttcgattcaagagatcgaagtattccgcgtacg

SEQ ID NO: 38 - DNA, 51 bp, Artificial
  Artificially synthesized oligonucleotide for siRNA
aaaacgtacgcggaatacttcgatctcttgaatcgaagtattccgcgtacg

SEQ ID NO: 39 - DNA, 47 bp, Artificial
  siRNA hairpin design
cgtacgcggaatacttcgattcaagagatcgaagtattccgcgtacg

SEQ ID NO: 40 - DNA, 51 bp, Artificial
  An artificially synthesized oligonucleotide for siRNA
tccccagcagaagctattcagacttcaagagagtctgaatagcttctgctg

SEQ ID NO: 41 - DNA, 51 bp, Artificial
  An artificially synthesized oligonucleotide for siRNA
aaaacagcagaagctattcagactctcttgaagtctgaatagcttctgctg

SEQ ID NO: 42 - DNA, 47 bp, Artificial
  siRNA hairpin design
cagcagaagctattcagacttcaagagagtctgaatagcttctgctg

SEQ ID NO: 43 - DNA, 51 bp, Artificial
  An artificially synthesized oligonucleotide for siRNA
tcccgccgtgctaacactgttacttcaagagagtaacagtgttagcacggc

SEQ ID NO: 44 - DNA, 51 bp, Artificial
  An artificially synthesized oligonucleotide for siRNA
aaaagccgtgctaacactgttactctcttgaagtaacagtgttagcacggc

SEQ ID NO: 45 - DNA, 47 bp, Artificial
  siRNA hairpin design
gccgtgctaacactgttacttcaagagagtaacagtgttagcacggc

SEQ ID NO: 46 - DNA, 51 bp, Artificial
  An artificially synthesized oligonucleotide for siRNA
tcccggtgattcacagagacatattcaagagatatgtctctgtgaatcacc

SEQ ID NO: 47 - DNA, 51 bp, Artificial
  An artificially synthesized oligonucleotide for siRNA
aaaaggtgattcacagagacatatctcttgaatatgtctctgtgaatcacc

SEQ ID NO: 48 - DNA, 47 bp, Artificial
  siRNA hairpin design
ggtgattcacagagacatattcaagagatatgtctctgtgaatcacc

SEQ ID NO: 49 - DNA, 33 bp, Artificial
  An artificially synthesized primer for PCR
cggggtaccccgacaaggcctgccgggagtagt

SEQ ID NO: 50 - DNA, 33 bp, Artificial
  An artificially synthesized primer for PCR
cccaagcttgggcgaatctgtgcagctcgtgtc

SEQ ID NO: 51 - DNA, 21 bp, Artificial
  An artificially synthesized primer for PCR
aacgtaggcatgtagaggctc

SEQ ID NO: 52 - DNA, 23 bp, Artificial
  An artificially synthesized primer for PCR
cggggaagaaagtgcttaaagga

SEQ ID NO: 53 - protein, 33 aa, Artificial
  An artificially synthesised dominant negative peptide
RRRRRRRRRRRGGGPSKKRTQSIQGKGKGKRSS

SEQ ID NO: 54 - protein, 34 aa, Artificial
  An artificially synthesised dominant negative peptide
RRRRRRRRRRRGGGERIYNISGNGSPLADSKEIF

SEQ ID NO: 55 - protein, 34 aa, Artificial
  An artificially synthesised dominant negative peptide
RRRRRRRRRRRGGGNIKKLSNRLAQICSSIRTHK

SEQ ID NO: 56 - DNA, 2410 bp, Homo sapiens
cggggaagaaagtgcttaaaggattgctcattgcaggatctcaagtttcccagcaggaactcgccatgcggggtcatgggggcgcagggacggagggaagggccttaccgtctgtcggccgtagggccaggggtaggagttctccttctgggccatccttagagagaaagggggaggagagctgggcggagaagggaaacagccgtgagaagcagagaaaaagagagagagagggaggggttggaccgatcgagccggaagcgctagcccgagctggtaaaagggagcaggtcagcacactagccccaatctgggcgctggtctcaccgcccccgccctgctatcgtccctacctccttccagccctgcggcgtgcgcgcaggccagcccaacggaccctctgatctacctgatcatctgcccactcccggcgcaaggcctgcgacaggaggccagctcacctggggtccaaggcactgctactctcccggccgcccgcaaacaactgaatctgccacgccgcgccctggccaaggacttttcaaatctcccgccccggccccattgggcaagctcgtcgcccatgcctagttcccattggctcgatgtcctgtgacgtaagtcatgggaacccaatggaagaggcggaatgagaagggctcacgcttggcttccagtttaggaaagtttccccaaattcccccagtgaattatcttgctttttcttttctttttttttttccttttttgagacaggtctctctctgtcgctcaggctggagtataatggtgctatctgggcttgctgcaacctctgcctcccgggttcaagcgattctcgtgcctcagccacccgagtagctgggattacaagcgcccgccaccacgcctggctaatttttgtatttttattagagacgggttttcgctgtgttggccaatctggtctcgaactcctgtcctcaggtgatccacccgcctcggcttcccaaaatgctgggattacaggcgtgagccaccgtgcccggccttcttctttttataaaaaaaattaaatctttaatgtaactggagtttatccttatgcatgtatgagctggggtctaacttctctgcccgatggagcgccagttatgtcaacaccatttgttaaacaaactgccccaacagaatttaaatgctgcccttgatatataatgttagcatatttacttgtgtctatttctagactttgtattccgttgtttgctcatttgcctgtgtgtttattcttgtgtcattactatgtcgttcttaaaagttctgacattcctgggaggagtttttccaccaatagacctttcgttgcaagatctctgttagctgccgtgttgtttgggcccactttcatttttcctccttcctgccacacttccttcttttctgctcttcctaaccctcagatttcattactgggttaaataaatacatttttgggccttctatgaaataaccagtggatatttattaagtatgtactgtccagcactcttctcctttcttctaacactctatttttcctttaggggaccacctgtctctccagtctatgcagtcggtttctgtagtagtttctgttgctgacaaccccaaatccctaggttgatgaagacctatttaaatctcacttttttttttttttttgagatgaagattcgctgtgttgcccaggctggagtgcaatggcacaatctcagctcaccacaacctcctcctccagggtgcaactgattctcctgtctcagcctcccgagtacctaggattataggtgcatgtgaccacgcccggctaatttttgtatttttagtagagacggggtttcatcacattgctcaggctggcctcaggtgatccgcccgccttggtctcccaaagtgcttggattacaggcgtgagccactgcgcccagctttattcccacttctgaaagcacttagagttttgaggatttgagaaggaaactgacttcattgcaggtccagaaatgagccccaattaacttatactaatcaataccttttattccagattatggttattggatcaggcataagcctgtgatttaagtgggccagctagtgggaatcagtttcttgctcagaatgctgggcagctcaaggaaacacttaacgtttactggtttatcataaagtgtattacaaatgatatagatgaaaagatgtatagggcaaggtatggaggaagagatttctggaagtgagcagggaagcatgcagccctcattgctgcttctagtgctactgaaggaagcagcttgacattgaagcctaacattgcagaaggcagaagagaggcctggaaagaacttcaggacttgatgacattgttgagcctctacatgcctacgtt

SEQ ID NO: 57 - DNA, 628 bp, Homo sapiens
gccacaaggcctgccgggagtagtagtttgtatagttcgcagccggccagggcggagccgggtgggttggtggggagggcggggaaacaagcctggaccacaactcccaggagtacccgcgacggcggcggcctcgctgtcgcactcaggctatagggggcgcattgggcggaagaccctgggccggcaggaacttgcttgtgattggatgttgtgggacggcggcttctcattcgtcagcctgtgactgtggagtttgaattgggtggcggttgactgtagagccgctctctctcactggcacagcgaggttttgctcagcccttgtctcgggaccgcaggtacgtgcctggcgacttcttcgggtggtccccgtccgccctcctcgtccctacccagtttcttgcttccctgccccatctccgccgctccccgcagcctccgccgagcgccatggctcctaggaagggcagtagtcgggtggccaagaccaactccttacggaggcggaagctcgcctcctttctgaaagacttcgaccgtgaaggtaaggggccaggccgtcgcggcctcctggggcggtcgcgggatgctggcgggtggggacacgagctgcacagattcgccc

SEQ ID NO: 58 - DNA, 19 bp, Artificial
  A target sequence for siRNA
gaagcagcacgacttcttc

SEQ ID NO: 59 - DNA, 19 bp, Artificial
  A target sequence for siRNA
ccaaactgctcaggcataa

SEQ ID NO: 60 - DNA, 19 bp, Artificial
  A target sequence for siRNA
acgcggcacttcacaattg

SEQ ID NO: 61 - RNA, 19 nt, Artificial
  An artificially synthesized oligonucleotide for siRNA
gaagcagcacgacuucuuc

SEQ ID NO: 62 - RNA, 19 nt, Artificial
  An artificially synthesized oligonucleotide for siRNA
ccaaacugcucaggcauaa

SEQ ID NO: 63 - RNA, 19 nt, Artificial
  An artificially synthesized oligonucleotide for siRNA
acgcggcacuucacaauug